SEQUENCE LISTING (28 sequences)

SEQ ID NO: 1 - DNA, 2796 bp, artificial sequence
  CDS (1)..(2796): fusion protein of human c-Met ECD and murine Fc region
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcaca

SEQ ID NO: 2 - DNA, 1758 bp, artificial sequence
  CDS (1)..(1758): human cMet extracellular Sema region and Flag-his-tag
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggactacaaggacgacgacgacaagcatgtccaccatcatcaccatcactgattcgaa

SEQ ID NO: 3 - DNA, 2823 bp, artificial sequence
  CDS (1)..(2823): recombinant protein of human c-Met ECD and his-tag
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacacaccatcatcaccatcactgattcgaa

SEQ ID NO: 4 - protein, 120 aa, Mus musculus
QVQLKQSGPGLVQPSQSLSITCTVSGFSLPNYGVHWVRQSPGKGLEWLGVIWSGGSTNYAAAFVSRLRISKDNSKSQVFFEMNSLQADDTAVYYCARNHDNPYNYAMDYWGQGTTVTVSS

SEQ ID NO: 5 - protein, 112 aa, Mus musculus
DIVLTQSPGSLAVYLGQRATISCRANKSVSTSTYNYLHWYQQKPGQPPKLLIYLASNLASGVPARFSGSGSGTDFTLNIHPLEEEDAATYYCQHSRDLPPTFGAGTKLELKR

SEQ ID NO: 6 - protein, 5 aa, Mus musculus
NYGVH

SEQ ID NO: 7 - protein, 16 aa, Mus musculus
VIWSGGSTNYAAAFVS

SEQ ID NO: 8 - protein, 12 aa, Mus musculus
NHDNPYNYAMDY

SEQ ID NO: 9 - protein, 15 aa, Mus musculus
RANKSVSTSTYNYLH

SEQ ID NO: 10 - protein, 7 aa, Mus musculus
LASNLAS

SEQ ID NO: 11 - protein, 9 aa, Mus musculus
QHSRDLPPT

SEQ ID NO: 12 - protein, 15 aa, artificial sequence
  PEPTIDE (1)..(15): optimized light chain CDR1
RADKSVSTSTYNYLH

SEQ ID NO: 13 - protein, 120 aa, artificial sequence
  PEPTIDE (1)..(120): Ab-9 heavy chain variable region
QVTLKESGPVLVKPTETLTLTCTVSGFSLPNYGVHWVRQPPGKALEWLAVIWSGGSTNYAAAFVSRLRISKDTSKSQVVFTMNNMDPVDTATYYCARNHDNPYNYAMDYWGQGTTVTVSS

SEQ ID NO: 14 - protein, 120 aa, artificial sequence
  PEPTIDE (1)..(120): Ab-10 heavy chain variable region
QVQLVESGGGVVQPGRSLRLSCAASGFSLSNYGVHWVRQAPGKGLEWLAVIWSGGSTNYAAAFVSRLTISKDNSKNTVYLQMNSLRAEDTAVYYCARNHDNPYNYAMDYWGQGTTVTVSS

SEQ ID NO: 15 - protein, 120 aa, artificial sequence
  PEPTIDE (1)..(120): Ab-11 heavy chain variable region
QVQLVESGGGVVQPGRSLRLSCAASGFTLPNYGVHWVRQAPGKGLEWLAVIWSGGSTNYAAAFVSRLTISKDNSKNTVYLQMNSLRAEDTAVYYCARNHDNPYNYAMDYWGQGTTVTVSS

SEQ ID NO: 16 - protein, 112 aa, artificial sequence
  PEPTIDE (1)..(112): Ab-9 light chain variable region
DIVLTQSPASLAVSPGQRATITCRANKSVSTSTYNYLHWYQQKPGQPPKLLIYLASNLASGVPARFSGSGSGTDFTLTINPVEANDTANYYCQHSRDLPPTFGQGTKLEIKR

SEQ ID NO: 17 - protein, 112 aa, artificial sequence
  PEPTIDE (1)..(112): Ab-10 light chain variable region
DIVLTQSPDSLAVSLGERATINCRADKSVSTSTYNYLHWYQQKPGQPPKLLIYLASNLASGVPDRFSGSGSGTDFTLTISSLQAEDVAVYYCQHSRDLPPTFGQGTKLEIKR

SEQ ID NO: 18 - protein, 112 aa, artificial sequence
  PEPTIDE (1)..(112): Ab-11 light chain variable region
DIVLTQSPDSLAVSLGERATINCRANKSVSTSTYNYLHWYQQKPGQPPKLLIYLASNLASGVPDRFSGSGSGTDFTLTISSLQAEDVAVYYCQHSRDLPPTFGQGTKLEIKR

SEQ ID NO: 19 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 446 aa, artificial sequence
  PEPTIDE (1)..(446): Ab-9 heavy chain
QVTLKESGPVLVKPTETLTLTCTVSGFSLPNYGVHWVRQPPGKALEWLAVIWSGGSTNYAAAFVSRLRISKDTSKSQVVFTMNNMDPVDTATYYCARNHDNPYNYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 446 aa, artificial sequence
  PEPTIDE (1)..(446): Ab-10 heavy chain
QVQLVESGGGVVQPGRSLRLSCAASGFSLSNYGVHWVRQAPGKGLEWLAVIWSGGSTNYAAAFVSRLTISKDNSKNTVYLQMNSLRAEDTAVYYCARNHDNPYNYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 446 aa, artificial sequence
  PEPTIDE (1)..(446): Ab-11 heavy chain
QVQLVESGGGVVQPGRSLRLSCAASGFTLPNYGVHWVRQAPGKGLEWLAVIWSGGSTNYAAAFVSRLTISKDNSKNTVYLQMNSLRAEDTAVYYCARNHDNPYNYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 218 aa, artificial sequence
  PEPTIDE (1)..(218): Ab-9 light chain
DIVLTQSPASLAVSPGQRATITCRANKSVSTSTYNYLHWYQQKPGQPPKLLIYLASNLASGVPARFSGSGSGTDFTLTINPVEANDTANYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 218 aa, artificial sequence
  PEPTIDE (1)..(218): Ab-10 light chain
DIVLTQSPDSLAVSLGERATINCRADKSVSTSTYNYLHWYQQKPGQPPKLLIYLASNLASGVPDRFSGSGSGTDFTLTISSLQAEDVAVYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 218 aa, artificial sequence
  PEPTIDE (1)..(218): Ab-11 light chain
DIVLTQSPDSLAVSLGERATINCRANKSVSTSTYNYLHWYQQKPGQPPKLLIYLASNLASGVPDRFSGSGSGTDFTLTISSLQAEDVAVYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2020/0054764 A1
(43) Pub. Date: Feb. 20, 2020

(54) MEDICAL USE OF ANTI-C MET ANTIBODY-CYTOTOXIC DRUG CONJUGATE

(71) Applicants: Suzhou Suncadia Biopharmaceuticals Co., Ltd., Jiangsu (CN); Jiangsu Hengrui Medicine Co., Ltd., Lianyungang, Jiangsu (CN); Shanghai Hengrui Pharmaceutical Co., Ltd., Minhang District, Shanghai (CN)

(72) Inventors: Xing SUN, Lianyungang, Jiangsu (CN); Guoqing CAO, Lianyungang, Jiangsu (CN); Mi TANG, Lianyungang, Jiangsu (CN); Jiahua JIANG, Lianyungang, Jiangsu (CN); Changyong YANG, Lianyungang, Jiangsu (CN); Lianshan ZHANG, Lianyungang, Jiangsu (CN)

(21) Appl. No.: 16/340,258

(22) PCT Filed: Oct. 13, 2017
(86) PCT No.: PCT/CN2017/106044

§ 371 Date: Apr. 8, 2019

(30) Foreign Application Priority Data
Oct. 14, 2016 (CN) 201610898963.7

(51) Int. Cl. A61K 47/68; A61K 38/08; C07K 16/28; A61K 31/40

(52) U.S. Cl. CPC A61K 47/6859; A61K 38/08; A61K 31/40; C07K 16/2863; A61K 47/6889

(57) ABSTRACT

The medical use of an anti-c Met antibody-cytotoxic drug conjugate is described. In particular, an anti-c-Met antibody, an antigen-binding fragment thereof, a chimeric antibody and a humanized antibody containing the anti-c-Met antibody CDRs, and an antibody-cytotoxic drug conjugate thereof or a pharmaceutically acceptable salt or solvate thereof are described. Also described are the use of a pharmaceutical composition containing the humanized anti-c-Met antibody, the antigen-binding fragment thereof, the antibody-cytotoxic drug conjugate thereof, or the pharmaceutically acceptable salt or solvate thereof as an anti-hepatoma drug.

### FIELD OF THE INVENTION

The present invention relates to the use of a c-Met antibody-cytotoxic drug conjugate or pharmaceutically acceptable salt or solvate thereof in the preparation of a medicament for treatment of hepatic carcinoma.

### BACKGROUND OF THE INVENTION

In recent years, molecular biology and tumor pharmacology studies have shown that tyrosine kinase (Protein Tyrosine Kinases, PTKs) related cell signaling pathways play an extremely important role in tumor formation and development, and that more than 50% of proto-oncogenes and oncogene products have tyrosine kinase activity. The c-Met proto-oncogene belongs to the Ron subfamily of the PTK family, and the encoded c-Met protein is a high affinity receptor for Hepatocyte Growth Factor/Scatter Factor (HGF/SF). The HGF/c-Met signaling pathway is closely related to the process of angiogenesis and tumor growth. The sustained activation of the pathway is an important cause of cancerization of tissue cells or of hyperproliferation of cancer cells. Inhibition of this pathway has become a new method of targeted tumor therapy.

The c-Met proto-oncogene, which is more than 120 kb in size, is located on the long arm of human chromosome 7 (7q31), and it encodes a c-Met protein precursor with a molecular weight of about 150 kD, which undergoes local glycosylation to form a 170 kD glycoprotein. The glycoprotein is further cleaved into a first subunit (50 kDa) and a second subunit (140 kDa), which are linked by disulfide bond to form a mature c-Met protein receptor. The heterodimer contains two strands, one comprises an extracellular domain, a transmembrane region (also called membrane stretch fragment), and an intracellular domain (comprising intracellular tyrosine kinase binding site). The other chain has only an extracellular portion, but it is highly glycosylated and is attached to the chain by disulfide bond. The extracellular region of the two subunits is the recognition site of the corresponding ligand, and the intracellular domain has tyrosine kinase activity.

C-Met activation occurs through three types of mechanism: one type depends on the activation mechanism of HGF, the second type does not depend on the HGF activation mechanism, and the third type occurs through other membrane pathways, such as through the hyaluronic acid surface receptor CD44, adhesin and RON signaling pathways, and so on. One of the most common mechanisms of c-Met activation is the one that is dependent on the activation mechanism of HGF. The N-terminus of HGF binds to c-Met to promote the dimerization and autophosphorylation of Tyr1234 and Tyr1235 on the chain, and phosphorylation of Tyr1349 and Tyr1356 near the C-terminus produces a binding site for multiple linker proteins which in turn induce P13K/Akt, Ras/Mapk, c-Src and STAT3/5-mediated activation of downstream signaling, and trigger different cellular responses, such as cell survival and activity (closely related to P13K/Akt pathway) and tumor metastasis and cell proliferation (mainly mediated by Ras/Mapk). In addition, the cross-talk of c-Met with other membrane receptors has been known to promote tumor formation and metastasis. Since c-Met is the intersection of many pathways leading to tumor formation and metastasis, simultaneously interfering with many pathways can be achieved relatively easily by targeting c-Met, and c-Met has become a promising target for antitumor formation and metastasis therapy.

An antibody drug conjugate (ADC) is formed by linking a monoclonal antibody or antibody fragment to a biologically active cytotoxin via a stable chemical linker, which fully utilizes the specificity of the antibody to a specific tumor cell or a highly expressed antigen, combined with the high efficiency of the cytotoxin, to avoid toxic side effects to normal cells. This means that antibody drug conjugates can bind tumor cells specifically and reduce their effects on normal cells, compared to conventional chemotherapeutic agents.

ADCs consist of three parts: antibodies (target), linkers and toxins. Among them, a good target (antibody portion), which includes not only specific targeting binding, but also effective endocytosis, determines the specificity of the ADC drug.

Currently, there are three main types of inhibitors for c-Met kinase targeting: HGF and c-Met biological antagonists, HGF and c-Met antibodies, and c-Met small molecule inhibitors. The existing clinical results show that the antibodies directly targeting HGF and c-Met, or c-Met small molecule inhibitors is not ideal. An ADC for c-Met may be the most effective method for treating a tumor. Presently, there is no c-Met ADC drug in clinical research.

PCT/CN2016/078699 to the present inventor discloses a type of c-Met ADC drug, and envisioned its use for treatment of cancer. However, the use for treatment of hepatic carcinoma was not suggested.

### SUMMARY OF THE INVENTION

The technical problem to be solved by the present invention is the use of an antibody-cytotoxic drug conjugate (ADC) or pharmaceutically acceptable salt or solvate thereof in the preparation of medicament for treatment of hepatic carcinoma, wherein said antibody-cytotoxic drug conjugate (ADC) is administered as the sole component which has prominent anti-tumor activity and inhibits the proliferation of hepatic carcinoma cells effectively, thus providing a better application in the clinic.

The technical solution of present invention is provided below: The present invention provides the use of an antibody-cytotoxic drug conjugate or pharmaceutically acceptable salt or solvate thereof in the preparation of medicament for treatment of hepatic carcinoma, wherein said antibody-cytotoxic drug conjugate has a structure of formula (I):

Ab-[(L₂)t-L₁-D)]y (I)

wherein:

D is cytotoxic drug;

L₁ and L₂ are linker units;

t is 0 or 1, preferably 1;

y is 1-8, preferably 2-5; and

Ab is an antibody or antigen-binding fragment thereof that specifically binds to c-Met receptor, comprising at least one CDR region sequence selected from the following sequences or mutant sequence thereof:
antibody heavy chain variable region HCDR sequence: SEQ ID NO: 6, SEQ ID NO:7 or SEQ ID NO:8; and
antibody light chain variable region LCDR sequence: SEQ ID NO: 9, SEQ ID NO: 10 or SEQ ID NO: 11.

Preferably, the antibody heavy chain variable region comprises at least one HCDR region sequence selected from the following sequences or mutant sequence thereof: SEQ ID NO: 6, SEQ ID NO: 7 and SEQ ID NO: 8.

Preferably, the antibody light chain variable region comprises at least one LCDR region sequence selected from the following sequences or mutant sequence thereof: SEQ ID NO: 9, SEQ ID NO: 10 and SEQ ID NO: 11.

In a preferred embodiment of the present invention, the antibody comprises heavy chain variable region sequences SEQ ID NO: 6, SEQ ID NO: 7 and SEQ ID NO: 8, or mutant sequence thereof, and light chain variable region sequences SEQ ID NO: 9, SEQ ID NO: 10 and SEQ ID NO: 11, or mutant sequence thereof.

The mutant sequences are sequences having 1-3 amino acid mutations in the CDRs that optimize antibody activity, wherein the mutant sequence of HCDR2 region is preferably SEQ ID NO: 12.

The antibody or antigen-binding fragment thereof that specifically binds to c-Met receptor is a murine antibody or fragment thereof.

The heavy chain variable region sequence of the murine antibody is shown as SEQ ID NO: 4.

The light chain variable region sequence of the murine antibody is shown as SEQ ID NO: 5.

In a preferred embodiment of the present invention, the heavy chain variable region of the murine antibody is shown as SEQ ID NO: 4, and the light chain variable region of the murine antibody is shown as SEQ ID NO: 5.

In a preferred embodiment of the present invention, the antibody or antigen-binding fragment thereof that specifically binds to c-Met receptor is a chimeric antibody or a humanized antibody or a fragment thereof.

The humanized antibody heavy chain variable region comprises a heavy chain FR region derived from human germline heavy chain sequence, preferably the human germline heavy chain IGHV 3-33*01; wherein said heavy chain FR region comprises the framework sequence of the FR1, FR2, FR3 and FR4 regions of human germline heavy chain IGHV 3-33*01, or a mutant sequence thereof, preferably the mutant sequence comprises 0-10 amino acid back-mutation(s).

The humanized antibody comprises a heavy chain variable region sequence selected from SEQ ID NOs: 13-15 or variants thereof.

The humanized antibody light chain variable region comprises a light chain FR region derived from human germline light chain sequence, preferably the human germline light chain IGKV085 or IGKV4-1*01; wherein said light chain FR region comprises the framework sequence of the FR1, FR2, FR3 and FR4 regions of human germline light chain IGKV085 and IGKV4-1*01, or mutant sequence thereof, preferably the mutant sequence comprises 0-10 amino acid back-mutation(s).

In a preferred embodiment of the present invention, the humanized antibody comprises a light chain variable region sequence selected from SEQ ID NOs: 16-18, or a variant thereof

In a preferred embodiment of the present invention, the humanized antibody comprises a heavy chain variable region sequence selected from SEQ ID NOs: 13-15 and a light chain variable region sequence selected from SEQ ID NOs: 16-18.

In a preferred embodiment of the present invention, said antibody or antigen-binding fragment thereof that specifically binds to c-Met receptor comprises a combination of heavy chain variable region sequence and light chain variable region sequence selected from any one of a) to c):

a) Heavy chain variable region sequence of SEQ ID NO: 13, and light chain variable region sequence of SEQ ID NO: 16;

b) Heavy chain variable region sequence of SEQ ID NO: 14, and light chain variable region sequence of SEQ ID NO: 17; or

c) Heavy chain variable region sequence of SEQ ID NO: 15, and light chain variable region sequence of SEQ ID NO: 18.

In a preferred embodiment of the present invention, the heavy chain constant region of the humanized antibody comprises a constant region derived from human IgG1 or a variant thereof, human IgG2 or a variant thereof, human IgG3 or a variant thereof, or human IgG4 or a variant thereof, preferably comprises a constant region derived from human IgG1 or a variant thereof, human IgG2 or a variant thereof, or human IgG4 or a variant thereof, more preferably a constant region derived from human IgG2 or a variant thereof.

In a preferred embodiment of the present invention, said antibody or antigen-binding fragment thereof that specifically binds to c-Met receptor comprises a full-length heavy chain sequence selected from SEQ ID NOs: 23-25 or sequences having at least 90% identity to SEQ ID NOs: 23-25.

In a preferred embodiment of the present invention, the light chain constant region of the humanized antibody comprises a constant region selected from human κ or λ, or a variant thereof.

The antibody or antigen-binding fragment thereof that specifically binds to c-Met receptor comprises a full-length light chain sequence selected from SEQ ID NOs: 26-28 or sequences having at least 90% identity to SEQ ID NOs: 26-28.

The humanized antibody comprises a combination of full-length light chain sequence and full-length heavy chain sequence selected from:

Ab-9: heavy chain sequence of SEQ ID NO: 23 and light chain sequence of SEQ ID NO: 26;

Ab-10: heavy chain sequence of SEQ ID NO: 24 and light chain sequence of SEQ ID NO: 27; or

Ab-11: heavy chain sequence of SEQ ID NO: 25 and light chain sequence of SEQ ID NO: 28.

The present invention further provides the use of a pharmaceutical composition in the preparation of a medicament for treatment of hepatic carcinoma, wherein said pharmaceutical composition comprises the c-Met antibody or antigen-binding fragment thereof described above and one or more pharmaceutically acceptable excipient, diluent or carrier.

In a preferred embodiment of the present invention, -L₂- is a compound shown as formula (-L₂-):

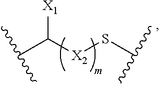

wherein:

X₁ is selected from the group consisting of hydrogen, halogen, hydroxyl, cyano, C₁₋₆ alkyl, C₁₋₆ alkoxy and 3-8 membered cycloalkyl;

X₂ is selected from the group consisting of C₁₋₆ alkyl, 3-8 membered cycloalkyl and 3-8 membered heterocyclyl;

m is 0-5, preferably 1-3; and

S is a sulfur atom.

Preferably, said cytotoxic drug unit of D is a cytotoxic agent selected from toxins, chemotherapeutic agents, antibiotics, radioisotopes and nucleolytic enzyme.

In a preferred embodiment of the present invention, D is a compound shown as formula (D):

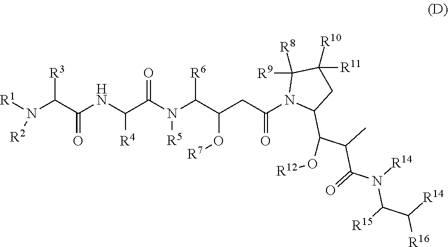

or tautomer, mesomer, racemate, enantiomer, diastereomer, or mixtures thereof, or pharmaceutically acceptable salt thereof:

wherein:

R¹, R², R³, R⁴, R⁵, R⁶, R¹ is each selected from the group consisting of hydrogen, halogen, hydroxyl, cyano, C₁₋₆ alkyl, C₁₋₆ alkoxy and 3-8 membered cycloalkyl;

R⁸, R⁹, R¹⁰, R¹¹ is each selected from the group consisting of hydrogen, halogen, C₂₋₆ alkenyl, C₁₋₆ alkyl, C₁₋₆ alkoxy and 3-8 membered cycloalkyl; preferably at least one group is selected from halogen, C₂₋₆ alkenyl, C₁₋₆ alkyl and 3-8 membered cycloalkyl, and the rest of the group(s) is(are) hydrogen,

or any two of R⁸, R⁹, R¹⁰, R¹¹ form a 3-8 membered cycloalkyl, and the remaining two are each selected from the group consisting of hydrogen, C₁₋₆ alkyl and 3-8 membered cycloalkyl;

R¹², R¹³ is each selected from the group consisting of hydrogen, C₁₋₆ alkyl and halogen;

R¹⁴ is selected from 6-14 membered aryl and 5-15 membered heteroaryl, wherein the aryl or heteroaryl is optionally further substituted by a substituent selected from the group consisting of hydrogen, halogen, hydroxy, C₁₋₆ alkyl, C₁₋₆ alkoxy and 3-8 membered cycloalkyl;

R¹⁵ is selected from the group consisting of halogen, C₂₋₆ alkenyl, C₁₋₆ alkyl, 3-8 membered cycloalkyl, carboxyl, C₁₋₆ alkyl carbonyl and C₁₋₆ alkoxy carbonyl; and

R¹⁶ is selected from the group consisting of hydrogen, halogen, hydroxy, cyano, alkyl, C₁₋₆ alkoxy and 3-8 membered cycloalkyl.

Preferably, L₂ comprises a linker selected from the group consisting of Val-Cit, MC, PAB and MC-PAB, preferably MC.

Particularly preferably, D is a maytansinoid; preferably DM1, DM3 or DM4; more preferably DM1.

Preferably, L₂ is selected from the group consisting of N-succinimidyl 4-(2-pyridylthio) valerate (SPP), N-succinimidyl 4-(N-maleimidomethyl)-cyclohexane-1-carboxylate (SMCC) and N-succinimidyl (4-iodo-acetyl) aminobenzoate (SIAB); preferably N-succinimidyl 4-(2-pyridylthio) valerate or N-succinimidyl 4-(N-maleimidomethyl)-cyclohexane-1-carboxylate.

Further preferably, D is a camptothecin alkaloid which is selected from the group consisting of CPT, 10-hydroxy-CPT, Irinotecan, SN-38 and topotecan, more preferably SN-38.

Particularly preferably, the linker L₂ is selected from the group consisting of Val-Cit, MC, PAB and MC-PAB; preferably MC or MC-vc-PAB.

In a preferred embodiment of the present invention, said antibody-cytotoxic drug conjugate is a conjugated drug of formula (II) or pharmaceutically acceptable salt or solvate thereof:

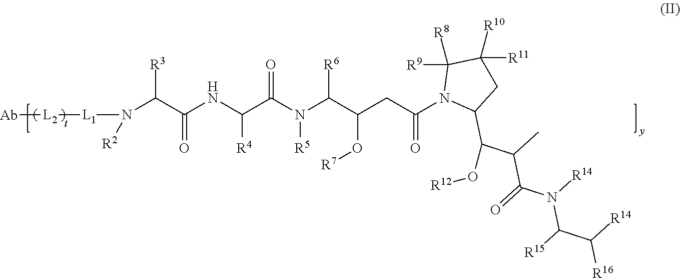

wherein:

R²-R¹⁶ are as defined in formula (D); and

Ab, t, y, L₁, and L₂ are as defined in formula (I).

In a preferred embodiment of the present invention, said antibody-cytotoxic drug conjugate is a conjugated drug of formula (III) or pharmaceutically acceptable salt or solvate thereof:

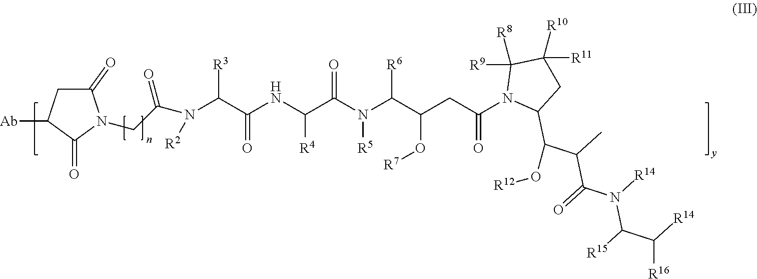

wherein:

R²-R¹⁶ are as defined in formula (D);

Ab and y are as defined in formula (I); and

n is 3-6, preferably 5.

In a preferred embodiment of the present invention, said antibody-cytotoxic drug conjugate is a conjugated drug of formula (IV) or pharmaceutically acceptable salt or solvate thereof:

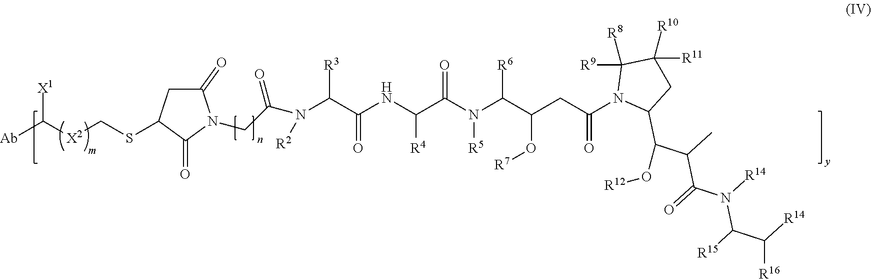

wherein:

R²-R¹⁶ are as defined in formula (D);

Ab and y are as defined in formula (I);

n is as defined in formula (III); and

X¹, X², and m are as defined in formula L₂.

In a preferred embodiment of the present invention, said antibody-cytotoxic drug conjugate is a conjugated drug of formula (V) or pharmaceutically acceptable salt or solvate thereof:

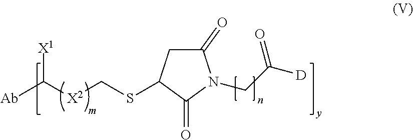

wherein:

Ab, D, and y are as defined in formula (I);

n is as defined in formula (III); and

X¹, X², and m are as defined in formula L₂.

In a preferred embodiment of the present invention, said antibody-cytotoxic drug conjugate or pharmaceutically acceptable salt or solvate thereof is selected from the group consisting of:

| No. | Structure and Name |
|---|---|
| 1 | 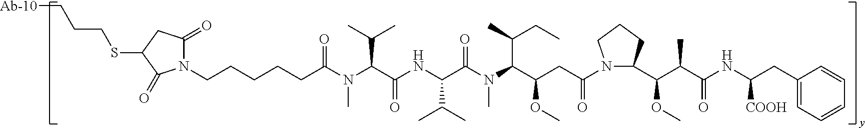 ADC-1 Anti c-Met antibody Ab-10 conjugated with toxin MC-MMAF |
| 2 | 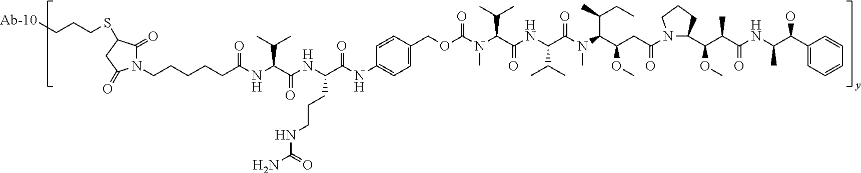 ADC-2 Anti c-Met antibody Ab-10 conjugated with toxin MC-VC-PAB-MMAE |
| 3 | 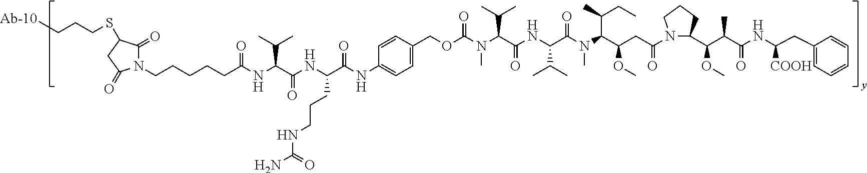 ADC-3 Anti c-Met antibody Ab-10 conjugated with toxin MC-VC-PAB-MMAF |
| 4 | 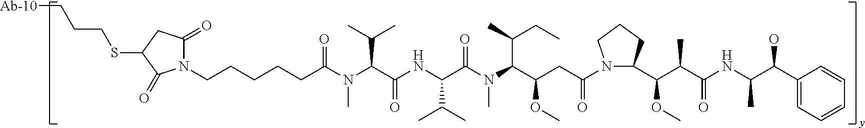 ADC-4 Anti c-Met antibody Ab-10 conjugated with toxin MC-MMAE |
| 5 | 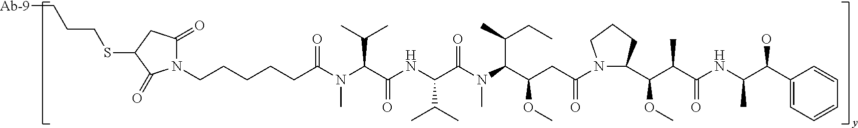 ADC-5 Anti c-Met antibody Ab-9 conjugated with toxin MC-MMAE |
| 6 | 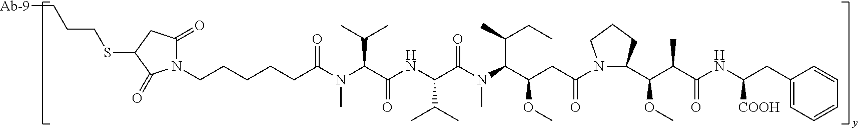 ADC-6 Anti c-Met antibody Ab-9 conjugated with toxin MC-MMAF |
| 7 | 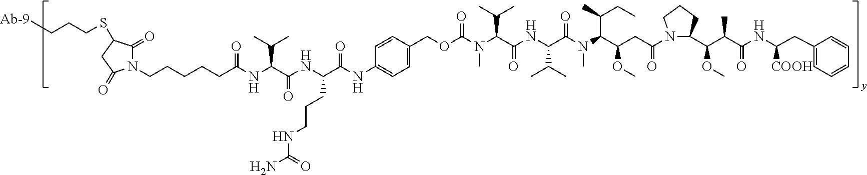 ADC-7 Anti c-Met antibody Ab-9 conjugated with toxin MC-VC-PAB-MMAF |
| 8 | 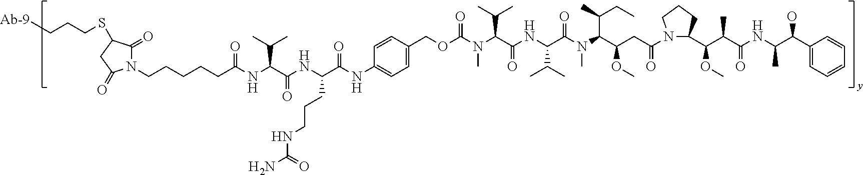 ADC-8 Anti c-Met antibody Ab-9 conjugated with toxin MC-VC-PAB-MMAE |
| 9 | 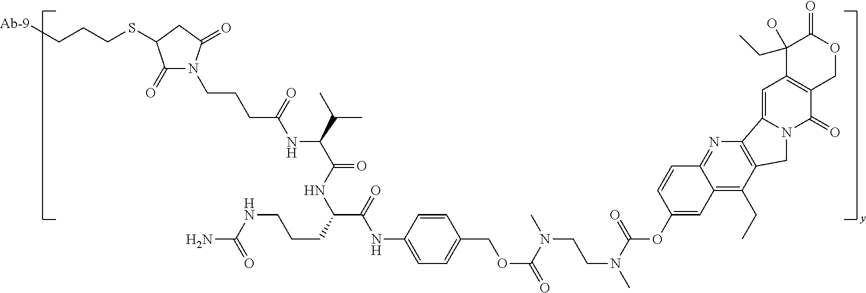 ADC-11 Anti c-Met antibody Ab-9 conjugated with toxin -SN-38 |
| 10 | 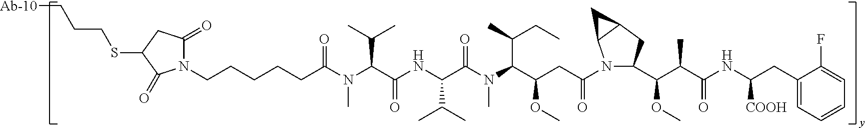 ADC-12 Anti c-Met antibody Ab-10 conjugated with toxin |
| 11 | 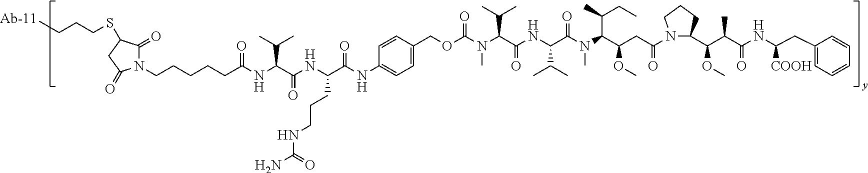 ADC-13 Anti c-Met antibody Ab-11 conjugated with toxin MC-VC-PAB-MMAF |
| 12 | 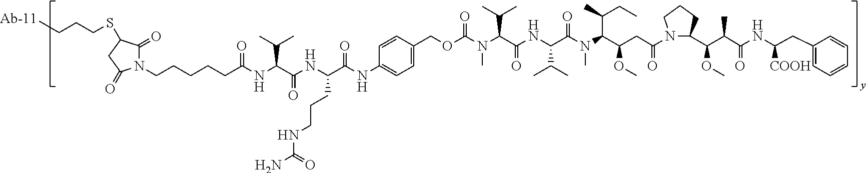 ADC-13 Anti c-Met antibody Ab-11 conjugated with toxin MC-MMAE |

wherein Ab-9, Ab-10, Ab-11 are c-Met antibodies as described above, and y is 1-8, preferably 2-5.

Wherein, y ranges from 1-8, preferably 1-4.

In a preferred embodiment of the present invention, the cancer cells of hepatic carcinoma are positive for c-Met expression or overexpresses c-Met, preferably ≥20% of hepatic carcinoma cells are positive/weak positive; more preferably ≥25% of hepatic carcinoma cells are positive; more preferably ≥50% of hepatic carcinoma cells are strongly positive.

### DETAILED DESCRIPTION OF THE INVENTION

### 1. Terms

In the specification and claims of present invention, unless specifically defined elsewhere in this document, the scientific and technical terms used herein have the meaning commonly understood by ordinary skilled in the art. However, in order to make the invention more readily understood, the definition and explanation of certain related terms are specifically provided below. Further, when the definition and explanation of the terms provided by the present application are inconsistent with the meanings generally understood by those skilled in the art, the definition and explanation of the terms provided by the present application shall prevail.

As used herein, the three-letter code and single-letter code for amino acids are as described in J. Biol. Chem, 243, p 3558 (1968).

The term “c-Met” or “c-Met polypeptide” or “c-Met receptor” refers to a receptor tyrosine kinase that binds to a hepatocyte growth factor (HGF). In the present invention, unless specified specifically, such as murine c-Met (m-c-Met) or monkey c-Met (cyno-c-Met), the term “c-Met” usually refers to human c-Met (h-c-Met). The human, murine and cynomolgus monkey c-Met used in the present invention are encoded by the nucleotide sequence or polypeptide sequence provided by GenBank, for example, the human polypeptide is encoded by the nucleotide sequence provided in GenBank Accession No. NM_000245, or the human protein or its extracellular domain have the polypeptide sequence provided in GenBank Accession No. NP_000236. The original single-stranded precursor proteins are cleaved after translation to produce alpha and beta subunits, which are linked by disulfide bonds to form mature receptors. The receptor tyrosine kinase c-Met is involved in cell processes including, for example, the process of migration, invasion and morphogenesis of tissue regeneration associated with embryogenesis.

The term “c-Met-related disorder or condition” refers to any disease, disorder or condition originating from adverse expression or lack of c-Met expression, adverse regulation or lack of regulation, or deleterious activity or lack of activity, or refers to any disease, disorder or condition which could be regulated, treated or cured by modulating c-Met expression or activity. The activation of the HGF/c-Met pathway can be expected, for example, in most cancer patients, or in patients whose disease is indeed driven by changes associated with the c-Met pathway. For example, upregulation is due to different mechanisms, such as overexpression of HGF and/or c-Met, or by constitutive activation of c-Met mutations. C-Met-related disorders or conditions include, but are not limited to, proliferative diseases and disorders and inflammatory diseases and disorders. Proliferative diseases include, but are not limited to, for example, cancer, including, for example, gastric cancer, esophageal cancer, breast cancer, kidney cancer including papillary renal cell carcinoma, lung cancer, glioma, head and neck cancer, epithelial cancer, skin cancer, leukemia, lymphoma, myeloma, brain cancer, pancreatic cancer, colorectal cancer, gastrointestinal cancer, intestinal cancer, genital cancer, urinary cancer, melanoma, prostate cancer, and other tumors known to those skilled in the art. Inflammatory diseases include, but are not limited to bacterial infections, including infections caused by Listeria bacteria.

“Antibody” in this invention refers to immunoglobulin, a four-peptide chain structure formed by two identical heavy chains and two identical light chains connected by interchain disulfide bonds. Different immunoglobulin heavy chain constant regions have different amino acid compositions and sequences, and thus present different kinds of antigenicity. Accordingly, immunoglobulins can be divided into five categories, also referred as immunoglobulin isotypes, namely IgM, IgD, IgG, IgA and IgE; the corresponding heavy chains thereof are chain, δ chain, γ chain, α chain, ε chain, respectively. According to the amino acid composition of the hinge region and the number and location of heavy chain disulfide bonds, immunoglobulins can be divided into different sub-categories, for example, IgG can be divided into IgG1, IgG2, IgG3, and IgG4. Light chains can be divided into κ or λ chains, based on different constant regions. Each category of Ig among these five categories involves a κ or λ chain.

Near the N-terminus of the antibody heavy and light chains, about 110 amino acids vary largely, and this region is known as the variable region (V region); the amino acid sequence near the C-terminus is relatively stable, and this region is known as the constant region (C region). The variable region comprises three hypervariable regions (HVR) and four framework regions (FR) with relatively conserved sequences. Three hypervariable regions determine the specificity of the antibody, also known as complementarity determining regions (CDRs). Each light chain variable region (LCVR) and each heavy chain variable region (HCVR) is composed of three CDR regions and four FR regions, arranged from the amino terminus to the carboxyl terminus as: FR1, CDR1, FR2, CDR2, FR3, CDR3, and FR4. The three light chain CDR regions are referred to as LCDR1, LCDR2, and LCDR3; the three heavy chain CDR regions are referred to as HCDR1, HCDR2 and HCDR3. The number and location of the CDR region amino acid residues in the LCVR and HCVR regions of the antibody or antigen binding fragment herein comply with the known Kabat numbering criteria (LCDR1-3, HCDE2-3), or comply with kabat and chothia numbering criteria (HCDR1).

The term “murine antibody” in the present invention refers to an anti-human c-Met monoclonal antibody prepared from mouse according to the knowledge and skills in the art. During the preparation, a test subject was injected with c-Met antigen, and then a hybridoma expressing the antibody possessing the desired sequence or functional characteristics was isolated. In a preferred embodiment of the present invention, the murine c-Met antibody or antigen binding fragment thereof, further comprises a light chain constant region of murine κ or λ chain, or a variant thereof, or further comprises a heavy chain constant region of murine IgG1, IgG2, IgG3 or IgG4, or a variant thereof.

The term “chimeric antibody” refers to an antibody that is obtained by fusing the variable region of a murine antibody to a constant region of a human antibody, wherein the chimeric antibody can alleviate the murine antibody-induced immune response. To establish a chimeric antibody, a hybridoma secreting a specific murine monoclonal antibody is first established, and the variable region gene is cloned from the murine hybridoma and then cloned into the constant region gene of a human antibody for recombinant expression.

The term “humanized antibody”, also known as humanized CDR-grafted antibody, refers to an antibody generated by grafting murine CDR sequences onto the framework of a human antibody variable region, that is to say, the antibodies are produced in different types of human germline antibody framework sequences. Humanized antibodies avoid the strong antibody immune response due to the chimeric antibody which carries a large number of murine protein components. The framework sequences can be obtained from public DNA databases covering germline antibody gene sequences or from published references. For example, germline DNA sequences of human heavy and light chain variable region genes can be found in the “VBase” human germline sequence database (available on the website www.mrccpe.com.ac.uk/vbase), as well as found in Kabat, E A, et al, 1991 Sequences of Proteins of Immunological Interest, 5th Ed. In a preferred embodiment of the invention, the murine CDR sequences of c-Met humanized antibody are selected from SEQ ID NOs: 6, 7, 8, 9, 10, and 11 (please check the # s, in case just copy from sost draft). Human antibody variable region frameworks were designed and selected, wherein the light chain FR region sequences of said antibody light chain variable regions are derived from human germline light chain sequences, preferably selected from human germline light chain IGKV085 or IGKV 4-1*01, comprising FR1, FR2, FR3 and FR4 regions of human germline light chain IGKV085 and IGKV 4-1*01; the heavy chain FR region sequences of said antibody heavy chain variable regions are derived from human germline heavy chain sequences, preferably selected from human germline heavy chain IGHV 3-33*01, comprising FR1, FR2, FR3 and FR4 regions of human germline heavy chain IGHV 3-33*01. To avoid a decrease of activity caused by a decrease of immunogenicity, a minimum of back mutation(s) could be introduced into a human antibody variable region to maintain the activity.

There are multiple methods available in the art to generate humanized antibodies. For example, humanized antibodies may be produced by obtaining HCVR and LCVR sequences of anti c-Met antibody (e.g., a murine antibody or antibody produced by a hybridoma), and grafting such sequences onto the selected human framework-encoding sequences. Optionally, a CDR region may be optimized by random mutagenesis or mutagenesis at particular locations in order to substitute one or more amino acids in the CDR with different amino acids prior to grafting the CDR region onto the framework region. Alternatively, a CDR region may be optimized after being inserted into the human framework region by using methods available to one of skilled in the art. Preferably, a “humanized antibody” has CDRs that originate from or are derived from a parent antibody (i.e., a non-human antibody, preferably a mouse monoclonal antibody), while framework and constant regions, to the extent they are present, (or a significant or substantial portion thereof, i.e., at least about 90%, 92%, 94%, 95%, 96%, 97%, 98% or 99%) are encoded by nucleic acids that occur in the human germline immunoglobulin region (see, e.g., the International ImMunoGeneTics Database) or in recombined or mutated forms thereof, regardless of whether said antibodies are produced in a human cell. Preferably, at least two, three, four, five or six CDRs of a humanized antibody are optimized from the CDRs of a non-human parent antibody from which the humanized antibody was derived, to generate a desired property, e.g., improved specificity, affinity or neutralization, which may be identified by a screening assay, e.g., an ELISA assay. Preferably, an optimized CDR in an antibody of the invention comprises at least one amino acid substitution when compared with that present in the parent antibody. When compared with CDRs of parent antibodies, certain amino acid substitutions in the CDRs of humanized antibodies of the invention (see example 6 herein) decrease the likelihood of instability of the antibody (e.g., removal of Asn residues from CDRs) or decrease the immunogenicity of the antibody when administered to a human subject (e.g., as predicted by IMMUNOFILTER™ Technology).

After the CDR-encoding sequences are grafted onto the selected human framework encoding sequences, the resultant DNA sequences encoding the humanized variable heavy and variable light chain sequences are then expressed to produce a humanized antibody that binds to c-Met. The humanized HCVR and LCVR may be expressed as part of a whole anti-c-Met antibody molecule, i.e., as a fusion protein with human constant domain sequences. However, the HCVR and LCVR sequences can also be expressed in the absence of constant sequences to produce a humanized anti-c-Met scFv.

References further describing methods involved in humanization of a mouse antibody that may be used include e.g., Queen et al., Proc. Natl. Acad. Sci. USA 88: 2869, 1991 and the method of Winter and co-workers [Jones et al., Nature, 321:522 (1986); Riechmann et al., Nature, 332:323-327 (1988); Verhoeyen et al., Science, 239:1534 (1988)].

“Antigen-binding fragment” in the present invention refers to a Fab fragment, a Fab′ fragment, or a F(ab′)2 fragment having antigen-binding activity, as well as an Fv fragment or an scFv fragment binding with human c-Met. It comprises one or more CDR regions of antibodies described in the present invention, selected from the group consisting of SEQ ID NO:3 to SEQ ID NO:8. An Fv fragment is a minimum antibody fragment comprising a heavy chain variable region, a light chain variable region, and all antigen-binding sites, without a constant region. Generally, an Fv antibody further comprises a polypeptide linker between the VH and VL domains, and is capable of forming a structure necessary for antigen binding. Also, different linkers can be used to connect the variable regions of two antibodies to form a polypeptide chain, referred to as a single chain antibody or a single chain Fv (scFv). An scFv can also be used with other antibodies such as an anti-EGFR antibody to construct a bispecific antibody. The term “binding with c-Met” used in this invention means being capable of interacting with human c-Met. The term “antigen-binding sites” in the present invention refers to discontinuous, three-dimensional sites on the antigen, recognized by the antibody or the antigen-binding fragment of the present invention. As used herein, the term “ADCC”, namely antibody-dependent cell-mediated cytotoxicity, means that the cells expressing Fc receptors directly kill the target cells coated by an antibody by recognizing the Fc segment of the antibody. ADCC effector function of the antibody can be reduced or eliminated by modifying the Fc segment in IgG. The modification refers to mutations performed on the antibody heavy chain constant region, such as mutations selected from N297A, L234A, L235A in IgG1; IgG2/4 chimera; F235E, and L234A/E235A mutations in IgG4.

As used herein, a fusion protein described in the present invention is a protein product obtained by co-expressing two genes via recombinant DNA technology. A recombinant c-Met extracellular domain Fc fusion protein is obtained by co-expressing a c-Met extracellular domain and a human antibody Fc fragment via recombinant DNA technology. The c-Met extracellular domain refers to the extracellular moiety of the c-Met protein.

The engineered antibody or antigen-binding fragment of the present invention may be prepared and purified using conventional methods. For example, cDNA sequences encoding a heavy chain (SEQ ID NO: 4) and a light chain (SEQ ID NO: 5) may be cloned and recombined into pEE6.4 expression vector (Lonza Biologics). The recombinant immunoglobulin expression vector may then be stably transfected into CHO cells. As a more recommended method well known in the art, mammalian expression system will make antibodies glycosylated, typically at the highly conserved N-terminus in the FC region. Stable clones may be obtained through expression of an antibody specifically binding to human c-Met. Positive clones may be expanded in a serum-free culture medium for antibody production in bioreactors. Culture medium, into which an antibody has been secreted, may be purified by conventional techniques. For example, the medium may be conveniently applied to a Protein A or G Sepharose FF column that has been equilibrated with a compatible buffer. The column is washed to remove nonspecific binding components. The bound antibody is eluted by PH gradient and the antibody fragments are detected by SDS-PAGE, and then collected. The antibody may be filtered and concentrated using common techniques. Soluble aggregate and multimers may be effectively removed by common techniques, including size exclusion or ion exchange. The obtained product may be immediately frozen, for example at −70° C., or may be lyophilized.

The term “antibody,” in this invention refers to a monoclonal antibody. As used herein, the term “monoclonal antibody” or “mAb” refers to an antibody secreted by a clone derived from a single cell strain. The cell strain is not limited to eukaryotic, prokaryotic, or phage clonal cell lines. Monoclonal antibodies or antigen-binding fragments can be obtained by recombinant methods, for example, hybridoma techniques, recombinant techniques, phage display techniques, synthetic techniques (such as CDR-grafting), or other techniques readily known in the art.

“Administration” and “treatment,” as they apply to an animal, human, experimental subject, cell, tissue, organ, or biological fluid, refer to contacting an exogenous pharmaceutical, therapeutic, diagnostic agent, or composition with the animal, human, subject, cell, tissue, organ, or biological fluid. “Administration” and “treatment” can refer, e.g., to therapeutic, pharmacokinetic, diagnostic, research, and experimental methods. Treatment of a cell encompasses contacting an agent with the cell, as well as contacting an agent with a fluid, where the fluid is in contact with the cell.

“Treat” means to administer a therapeutic agent, such as a composition comprising any of the binding compounds of the present invention, internally or externally to a patient having one or more disease symptoms for which the agent has known therapeutic activity. Typically, the therapeutic agent is administered in an amount effective to alleviate one or more disease symptoms in the treated patient or population, by inducing the regression of or inhibiting the progression of such symptom(s) to any clinically measurable degree. The amount of a therapeutic agent that is effective to alleviate any particular disease symptom (also referred to as “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the disease symptom(s) of interest in every patient, it can alleviate the target disease symptom(s) of interest in a statistically significant number of patients as determined by any statistical test known in the art such as the Student's t-test, the chi-square test, the U-test according to Mann and Whitney, the Kruskal-Wallis test (H-test), Jonckheere-Terpstra-test and the Wilcoxon-test.

“Conservative modification” or “conservative replacement or substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those skilled in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4.th Ed.)). In addition, substitutions of structurally or functionally similar amino acids are less likely to disrupt biological activity.

The term “consisting essentially of” or variations thereof as used throughout the specification and claims, indicates the inclusion of any of the recited elements or group of elements, and optionally inclusion of other elements, of similar or different nature than the recited elements, which do not significantly change the basic or novel properties of the specified dosage regimen, method, or composition. As a non-limiting example, a binding compound which consists essentially of a recited amino acid sequence may also include one or more amino acids that do not significantly affect the properties of the binding compound.

“Effective amount” encompasses an amount sufficient to ameliorate or prevent a symptom or sign of a medical condition. Effective amount also refers to an amount sufficient to allow or facilitate diagnosis. An effective amount for a particular patient or veterinary subject may vary depending on factors such as the condition being treated, the general health of the patient, the route and dose of administration and the severity of side effects. An effective amount can be the maximal dose or dosing regimen that avoids significant side effects or toxic effects.

“Exogenous” refers to substances that are produced outside an organism, cell, or human body, depending on the context. “Endogenous” refers to substances that are produced within a cell, organism, or human body, depending on the context.

“Homology” refers to sequence similarity between two polynucleotide sequences or between two polypeptides. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions to be compared and then multiplied by 100. For example, if 6 of 10 positions in two sequences are matched or homologous when the sequences are optimally aligned, then the two sequences are 60% homologous. Generally, the comparison is made when two sequences are aligned to give maximum percent homology.

“Optional” or “optionally” means that the event or situation that follows may but does not necessarily occur, and the description includes the instances in which the event or situation does or does not occur. For example, “optionally comprises 1-3 antibody heavy chain variable regions” means that the antibody heavy chain variable region with specific sequence can be, but is not necessarily present.

“Pharmaceutical composition” refers to a mixture comprising one or more compounds according to the present invention or physiologically/pharmaceutically acceptable salt or prodrug thereof with other chemical components, as well as additional components such as physiologically/pharmaceutically acceptable carriers and excipients. The pharmaceutical composition aims at promoting the administration to an organism, facilitating the absorption of the active ingredient and thereby exerting a biological effect.

Preparation of conventional pharmaceutical compositions can be found in Chinese pharmacopoeia.

The term “carrier” is applied for the drug of the present invention, and refers to a system that can change the manner in which a drug enters into the human body, and change the in vivo distribution, control the release rate of the drug, and delivery of the drug to the target organ. Drug carrier releasing and targeting systems are capable of reducing drug degradation and loss, decreasing side effects, and improving bioavailability. For example, a macromolecular surfactant used as a carrier can be self-assembled to form aggregates in various forms because of its unique amphiphilic structure, and preferred examples include micelles, emulsions, gels, liquid crystals, vesicles, etc. These aggregates not only have the ability to entrap drug molecules, but also display good membrane permeability, and can be used as excellent drug carriers.

The term “diluent” is also referred to as filler, and its main purpose is to increase the weight and volume of the tablet. The addition of diluent is not only to ensure a certain volume, but also to reduce the dose deviation of the main components and to improve the compression moldability of the drug. When pharmaceutical tablets contain an oil component, an absorbent must be added to absorb the oil material, and maintain the “dry” state, which facilitates tablet formation.

The term “pharmaceutically acceptable salt” refers to a salt form of a ligand-cytotoxic drug conjugate of the present invention, wherein the salt is safe and effective, and has the desired biological activity in mammals in vivo. The antibody-drug conjugate compound of the present invention comprises at least one amino group, by which the antibody-drug conjugate compound can form a salt with acid, including salt formed with inorganic or organic acids, such as carboxylic acid etc.

The term “solvate” refers to a pharmaceutically acceptable solvate formed by a ligand-drug conjugate of the present invention with one or more solvent molecule(s).

The term “ligand” is a macromolecular compound which is able to recognize and bind to the target cell-associated antigens or receptors. The role of the ligand is to deliver the drug to the target cell population bound to the ligand. The ligand includes, but is not limited to, proteinaceous hormones, lectins, growth factors, antibodies and other molecules capable of binding to cells.

The therapeutic agent is a molecule or atom that is administered separately, simultaneously or successively with a binding moiety, such as an antibody or antibody fragment, or sub-fragment thereof, and is useful for the treatment of the disease. Examples of therapeutic agents include, but are not limited to, antibodies, antibody fragments, conjugates, drugs, cytotoxic agents, apoptotic agents, toxins, nucleases (including DNase and RNase), hormones, immunomodulators, chelating agents, Boron compounds, photosensitizers or dyes, radioisotopes or radionuclides, oligonucleotides, interfering RNAs, peptides, antiangiogenic agents, chemotherapeutic agents, cytokines, chemokines, prodrugs, enzymes, binding proteins or peptides, or combination thereof.

The conjugate is an antibody component or other targeting moiety conjugated to a therapeutic agent as described above. As used herein, the terms “conjugate” and “immunoconjugate” are used interchangeably.

The term “cytotoxic agent” as used herein refers to a substance that inhibits or prevents the function of the cell and/or causes cell death or destruction.

“Toxin” refers to any substance capable of adversely affecting cell growth or proliferation.

“Chemotherapeutic agent” refers to a chemical compound that can be used to treat cancer. The definition also includes anti-hormonal agents that regulate, reduce, block or inhibit the effects of hormones that promote cancer growth, and chemotherapeutic agents are often used for systemic treatment. They can be hormones.

Auristatins are completely synthetic drugs with a relatively easily modified chemical structure that facilitates the optimization of physical properties and drug features. Auristatin derivatives used for antibody conjugation include monomethyl auristatin E (MMAE) and monomethyl auristatin F (MMAF). MMAE is a synthetic penta-peptide derived from natural tubulin polymerase inhibitor dolastatin-10, synthesized by adding 2-amino-1-phenylpropyl-1-ol at the C-terminus. The inhibitory activities of MMAE against a variety of human tumor cell lines are less than one nanomolar. In order to reduce the cytotoxic activity of MMAE itself, a phenylalanine is introduced at the C-terminus of dolastatin-10 in the case of MMAF. Due to the introduction of a carboxyl group in the structure, MMAF has poor membrane permeability, and therefore the biological activity against cells is significantly decreased, but the inhibitory activity against cells is increased substantially after it is conjugated to an antibody (U.S. Pat. No. 7,750,116).

The term “tubulin inhibitor” refers to a class of compounds that exert an anti-tumor effect by inhibiting or promoting polymerization of tublin, and consequently interfering with the cell mitosis process. Non-limiting examples include maytansines, calicheamicins, taxanes, vincristines, colchicines, and Dolastatins/Auristatins, preferably maytansines or Dolastatins/Auristatins; more preferably compounds of formula Di or DM.

CPT is short for camptothecin, and in this application CPT is used to refer to camptothecin itself or analogs or derivatives of camptothecin. The structures of camptothecin having the indicated number and the rings labeled with the letters A-E and some analogs thereof are provided in the following formula.

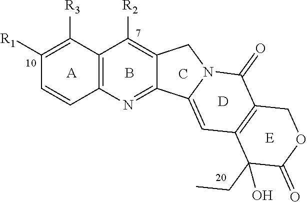

CPT: R₁=R₂=R₃=H

10-hydroxy-CPT:R₁=OH; R₂=R₃=H

Irinotecan: R₁=

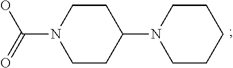 [SMILES: O=C(O)N1CCC(N2CCCCC2)CC1]

R₂=ethyl; R₃=H

SN-38: R₁=OH; R₂=ethyl; R₃=H

Topotecan: R₁=OH; R₂=H; R₃=CH—N(CH₃)₂

The term “intracellular metabolite” refers to a compound produced by intracellular metabolic processes or reactions of antibody-drug conjugates (ADCs). The metabolic process or reaction may be an enzymatic process, such as proteolytic cleavage of a peptide linker of an ADC, or hydrolysis of a functional group such as a hydrazone, ester or amide. Intracellular metabolites include, but are not limited to, antibodies and free drugs that undergo intracellular cleavage after entering, diffusing, ingesting or transporting into cells.

The terms “of intracellular cleavage” and “intracellular cleavage” refer to intracellular metabolic processes or reactions of antibody-drug conjugates (ADCs), wherein the covalent attachment between drug moiety (D) and antibody (Ab) is cleaved (i.e. the linker is cleaved), resulting in intracellular dissociation of free drug from the antibody. The module cleaved from ADC is thus an intracellular metabolite.

The term “bioavailability” refers to the systemic availability (i.e., blood/plasma level) of a given amount of drug administered to a patient. Bioavailability is an absolute term that indicates the time (rate) and the total amount (degree) required by the drug to achieve systemic circulation from the administered dose.

The term “cytotoxic activity” refers to cell killing, cytostatic, or growth inhibitory effects of intracellular metabolites of antibody-drug conjugates. Cytotoxic activity can be expressed as the IC50 value, that is, the concentration (molar or mass) per unit volume when half of cells survive.

The “C₁₋₆ alkyl” described in the present invention refers to a linear or branched alkyl group having 1 to 6 carbon atoms, and includes, for example, “C₁₋₄ alkyl”, “C₁₋₃ alkyl” etc., specific examples include but are not limited to methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl and the like.

The “C₂₋₆ alkenyl” described in the present invention refers to a linear, branched or cyclic alkenyl group having at least one double bond and having 2 to 6 carbon atoms, and includes for example “C₂₋₄ alkenyl group” and the like. Examples include, but are not limited to, vinyl, 1-propenyl, 2-propenyl, 1-butenyl, 2-butenyl, 1,3-butadienyl, 1-pentenyl, 2-pentenyl, 3-pentenyl, 1,3-pentadienyl, 1,4-pentadienyl, 1-hexenyl, 2-hexenyl, 3-hexenyl, 1,4-hexadienyl, cyclopentenyl, 1,3-cyclopentadienyl, cyclohexenyl, 1,4-cyclohexadienyl and the like.

The “3-8 membered cycloalkyl” described in the present invention refers to a saturated cyclic alkyl group having 3 to 8 carbon atoms, and includes, for example, “3-6 membered cycloalkyl” and “5-6 membered cycloalkyl” etc. Specific examples include, but are not limited to, cyclopropyl, cyclobutyl, cyclopentyl, cyclohexyl, cycloheptyl, cyclooctyl, and the like. The “5-6 membered cycloalkyl” refers to a saturated cyclic alkyl group having 5 to 6 carbon atoms.

The “C₁₋₆ alkoxy” described in the present invention refers to a group which is linked in a form of C₁₋₆ alkyl-O—, wherein “C₁₋₆ alkyl” is as defined above.

The term “bond” refers to a covalent bond presented as “—”.

The term “Hydroxy” refers to an —OH group.

The term “Halogen” refers to fluoro, chloro, bromo or iodo atoms, etc.

The term “Amino” refers to an —NH₂ group.

The term “Cyano” refers to a —CN group.

The term “Nitro” refers to a —NO₂ group.

The term “Oxo group” refers to a ═O group.

The “3-8 membered heterocyclic group” described in the present invention refers to a cyclic group having 3 to 8 ring atoms (at least one of which is a hetero atom such as a nitrogen atom, an oxygen atom or a sulfur atom). Optionally, a ring atom (e.g., a carbon atom, a nitrogen atom, or a sulfur atom) in the cyclic structure can be oxidized. A “5-6 membered heterocyclic group” is preferred. Specific examples include, but are not limited to, azacyclopropyl, 2H-azacyclopropyl, diazacyclopropyl, 3H-diazacyclopropenyl, azacyclobutyl, 1,4-dioxoheterocyclohexyl, 1,3-dioxoheterocyclohexyl, 1,3-dioxoheterocyclopentyl, 1,4-dioxoheterocyclodiallyl, tetrahydrofuranyl, dihydropyrrolyl, pyrrolidinyl, pyrrolidine-2,5-dione, imidazolidinyl, 4,5-dihydroimidazolyl, pyrazolidinyl, 4,5-dihydropyrazolyl, 2,5-dihydrothiophenyl, tetrahydrothiophenyl, 4,5-dihydrothiazolyl, thiazolidinyl, piperidinyl, tetrahydropyridyl, piperidinone, tetrahydropyridinone, dihydropyridinone, piperazinyl, morpholinyl, 4,5-dihydrooxazolyl, 4,5-dihydroisoxazolyl, 2,3-dihydroisoxazolyl, oxazolidinyl, 2H-1,2-oxazinyl, 6H-1,3-oxazinyl, 4H-1,3-thiazinyl, 6H-1,3-thiazinyl, 2H-pyranyl, 2H-pyranyl-2-one, 3,4-dihydro-2H-pyranyl and the like. The “5-6 membered heterocyclic group” refers to a particular example of 3-8 membered heterocyclic group which comprises 5 to 6 ring atoms.

The “6-8 membered aryl” described in the present invention refers to a monocyclic aryl group having 6 to 8 ring carbon atoms, and examples include, but are not limited to, phenyl, cyclooctatetraenyl, and the like.

The “6-15 membered fused aryl” described in the present invention refers to an unsaturated aromatic cyclic group having 6 to 15 ring carbon atoms, which is formed by two or more cyclic structures sharing two adjacent atoms with each other. Specific examples include, but are not limited to, naphthyl, anthryl, phenanthryl and the like. The “6-10 membered fused aryl” refers to a specific example of 6-14 membered fused aryl, which has 6 to 10 ring atoms.

The “5-8 membered heteroaryl” described in the present invention refers to an aromatic cyclic group having 5 to 8 ring atoms (in which at least one ring atom is hetero atom, such as a nitrogen atom, an oxygen atom or a sulfur atom). Optionally, the ring atom in the cyclic structure (e.g., a carbon atom, a nitrogen atom or a sulfur atom) can be oxidized. A “5-6 membered heteroaryl” is preferred. Specific examples include, but are not limited to, furyl, thienyl, pyrrolyl, thiazolyl, isothiazolyl, thiadiazolyl, oxazolyl, isoxazolyl, oxadiazolyl, imidazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, pyridyl, 2-pyridinone, 4-pyridinone, pyrimidinyl, pyridazinyl, pyrazinyl, 1,2,3-triazinyl, 1,3,5-triazinyl, 1,2,4,5-tetrazinyl, azacycloheptatrienyl, 1,3-diazacycloheptatrienyl, azacyclooctatetraenyl and the like. The “5-6 membered heteroaryl” refers to a specific example of 5-8 membered heteroaryl, which has 5 to 6 ring atoms.

The “carbon atom, nitrogen atom or sulfur atom is oxidized” described in the present invention refers to a structure in which C═O, N═O, S═O or SO₂ is formed.

The term “optional” or “optionally” means that the event or circumstance described subsequently can, but does not necessarily, occur, and the description includes the instances in which the event or circumstance does or does not occur. For example, “the heterocyclic group optionally substituted with an alkyl” means that an alkyl group can be, but is not necessarily, present, and the description includes a case wherein the heterocyclic group is substituted with an alkyl and a case wherein the heterocyclic group is not substituted with an alkyl.

“Substituted” refers to one or more hydrogen atoms in the group, preferably up to 5, more preferably 1 to 3 hydrogen atoms, each independently substituted with the corresponding number of substituents. It is clear that the substituents only occur in their possible chemical position. The person skilled in the art is able to determine if the substitution is possible or impossible without paying excessive efforts by experiment or theory. For example, the conjugation between amino or hydroxy group having free hydrogen and carbon atoms having unsaturated bonds (such as alkene) may be unstable.

“Linker or linker unit” refers to a chemical module comprising a covalent or atomic chain that covalently attaches the antibody to the drug module. In various embodiments, the linker includes: divalent radicals such as alkyldiyl, arylene, heteroarylene, such as unit like —(CR₂)ₙO (CR₂)ₙ—, hydrocarbyloxy repeat units (e.g., polyethyleneamino, PEG, polymethyleneoxy) and aminoalkyl (e.g., polyvinylamino, Jeffamine™), and the like; and diesters and amides including succinate, succinamide, bis-glycolate, malonate and caproamide.

Abbreviations:

Linker Units:

MC=6-maleimido-caproyl

Val-Cit or “vc”=valine-citrulline (an exemplary dipeptide of a protease cleavable linker)

Citrulline=2-Amino-5-ureido pentanoic acid

PAB=p-aminobenzyloxycarbonyl (examples of “self-immolative” linker unit)

Me-Val-Cit=N-methyl-valine-citrulline (wherein the linker peptide bond has been modified to prevent from being cleaved by cathepsin B)

MC(PEG)₆-OH=maleimido-caproyl-polyethylene glycol (which can be attached to antibody cysteine)

SPP=N-Succinimidyl 4-(2-pyridylthio) valerate

SPDP=N-Succinimidyl 3-(2-pyridyldithio) propionate

SMCC═Succinimidyl-4-(N-maleimidomethyl) cyclohexane-1-carboxylate

IT=imino sulfane

Cytotoxic drugs:

MMAE=Monomethyl auristatin E (MW 718)

MMAF=variant of auristatin E (MMAE), which has phenylalanine at the C-terminus of the drug (MW731.5)

MMAF-DMAEA=DMAEA (dimethylaminoethylamine) linked to the phenylalanine at C-terminus of MMAF (MW 801.5) via amide

MMAF-TEG=tetraethylene glycol is esterified to phenylalanine of MMAF

MMAF-NtBu=N-tert-butyl as an amide attached to the C-terminus of the MMAF

DM1=N(2′)-deacetyl-N(2′)-(3-mercapto-1-oxopropyl)-maytansine

DM3=N(2′)-deacetyl-N2-(4-mercapto-1-oxopentyl)-maytansine

DM4=N(2′)-deacetyl-N2-(4-mercapto-4-methyl-1-oxopentyl)-maytansine

The present invention also provides an antibody-cytotoxic drug conjugate comprising any anti-c-Met antibody of the invention or other c-Met antibody showing endocytosis activity (e.g., LY-2875358) conjugated to one or more cytotoxic agents, or pharmaceutically acceptable salt or solvate thereof (interchangeable as “antibody-drug conjugate” or “ADC”), wherein the cytotoxic agents include, for example, chemotherapeutic agents, drugs, growth inhibitors, toxins (e.g., bacterial, fungal, plant or animal-derived enzyme-active toxins or fragments thereof) or radioisotopes (i.e., radio-conjugates).

In certain embodiments, the antibody-cytotoxic drug conjugate or pharmaceutically acceptable salt or solvate thereof comprises an anti-c-Met antibody and a chemotherapeutic agent or other toxin. The chemotherapeutic agents that can be used to produce an antibody-cytotoxic drug conjugate or pharmaceutically acceptable salt or solvate thereof have been described herein (described above). Enzyme-active toxins and fragments thereof are also used, which are described in the specification.

In certain embodiments, the antibody-cytotoxic drug conjugate or pharmaceutically acceptable salt or solvate thereof comprises an anti-c-Met antibody and one or more small molecule toxins including, but not limited to small molecule drugs such as camptothecin derivatives, calicheamicin, maytansinoids, dolastatin, oricotine, trichothecene and CC1065, and cytotoxic fragments of these drugs.

Exemplary L₂ linkers include 6-maleimidocaproyl (“MC”), maleimidopropionyl (“MP”), valine-citrulline (“val-cit” or “vc”), alanine-phenylalanine (ala-phe), p-aminobenzyloxycarbonyl (“PAB”), N-succinimidyl 4-(2-pyridylthio) pentanoate “SPP”), N-succinimidyl 4-(N-maleimidomethyl) cyclohexane-1 carboxylate (“SMCC”), and N-succinimidyl (4-iodo-acetyl) aminobenzoate (“SIAB”). A variety of linkers are known in the art and are described below.

The linker may be a “cleavable linker” that facilitates the release of the drug in the cell. For example, an acid-labile linker (e.g., hydrazone), a protease-sensitive (e.g., peptidase-sensitive) linker, a light-labile linker, a dimethyl linker, or disulfide-containing linker may be used (Chari et al, Cancer Research 52: 127-131(1992); U.S. Pat. No. 5,208,020).

In some embodiments, the linker element may be a “stretcher unit” that connects the antibody to another linker element or drug module. Exemplary stretcher units are shown below (where the wavy line indicates the site to which the antibody is covalently attached):

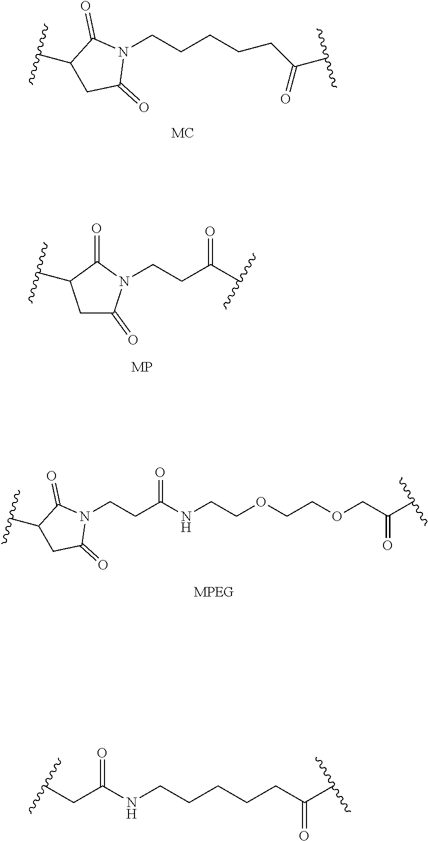 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, the linker unit may be an amino acid unit. In one such embodiment, the amino acid unit allows the linker to be cleaved by the protease, thereby facilitating release of the drug from the antibody-cytotoxic drug conjugate or its pharmaceutically acceptable salt or solvate after exposure to intracellular proteases, such as lysosomal enzymes. See for example Doronina et al (2003) Nat. Biotechnol. 21: 778-784. Exemplary amino acid units include, but are not limited to, dipeptides, tripeptides, tetrapeptides, and pentapeptides. Exemplary dipeptides include: valine-citrulline (VC or val-cit); alanine-phenylalanine (AF or ala-phe); phenylalanine-lysine (FK or phe-lys); or N-Methyl-valine-citrulline (Me-val-cit). Exemplary tripeptides include glycine-valine-citrulline (gly-val-cit) and glycine-glycine-glycine (gly-gly-gly). The amino acid units may comprise naturally occurring amino acid residues, as well as minor amino acids and non-naturally occurring amino acid analogs, such as citrulline. Amino acid units can be designed and optimized for their selectivity to enzymatic cleavage by specific enzymes, such as tumor-associated proteases, cathepsin B, C or D, or plasma proteases.

In some embodiments, the linker unit may be a “spacer” unit that connects the antibody (either directly or through the stretcher unit and/or the amino acid unit) to the drug module. The spacer unit may be “self-immolative” or “non-self immolative”.

The “non-self immolative” spacer unit refers to a spacer unit of which portion or the whole remains bound to the drug module after enzymatic cleavage (protein hydrolysis) of the ADC. Examples of non-self immolative spacer units include, but are not limited to, glycine spacer units and glycine-glycine spacer units. Other combinations of peptide spacers susceptible to sequence-specific enzymatic cleavage are also contemplated. For example, enzymatic cleavage of glycine-glycine spacer unit-containing ADC by tumor-cell-associated protease will result in the release of the glycine-glycine-drug module from the remainder of the ADC. In one such embodiment, the glycine-glycine-drug module is then subjected to a separate hydrolysis step in the tumor cells, thereby the glycine-glycine spacer unit is cleaved from the drug module.

The “self-immolative” spacer unit allows the release of the drug module without separate hydrolysis steps. In certain embodiments, the spacer unit of the linker comprises a p-aminobenzyl unit. In one such embodiment, p-aminobenzyl alcohol is attached to the amino acid unit via an amide bond, thereby forming a carbamate, methyl carbamate, or carbonate between benzyl alcohol and the cytotoxic agent. See, for example, in Hamann et al, (2005) Expert Opin. Ther. Patents (2005) 15: 1087-1103. In one embodiment, the spacer unit is p-aminobenzyloxycarbonyl (PAB).

Exemplary linkers in the present invention are as follows:

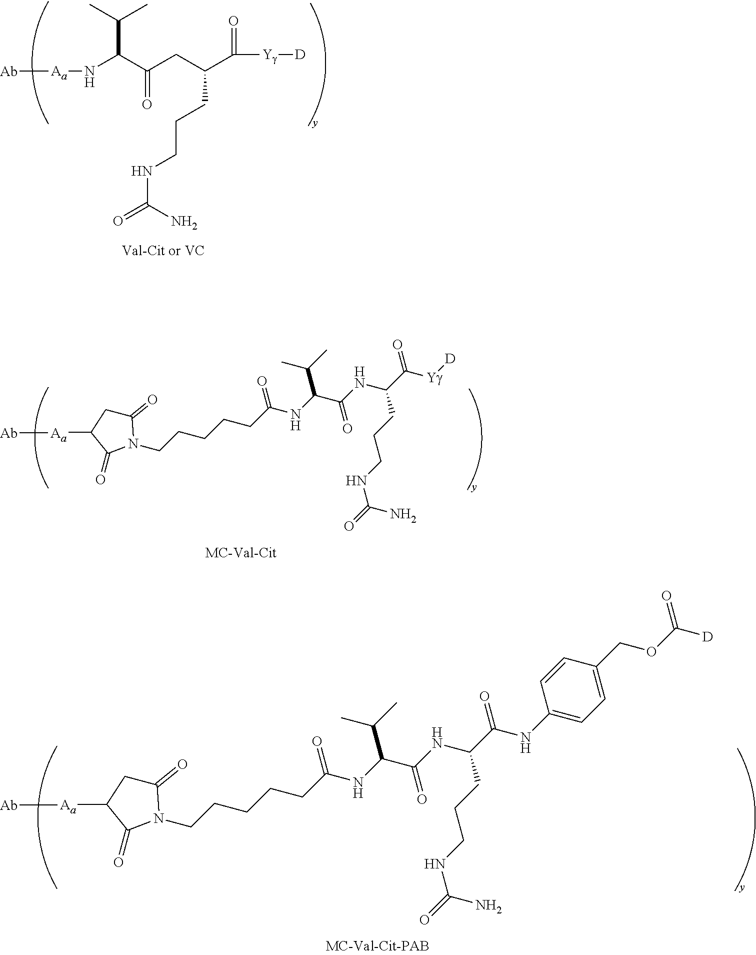

The linker, including stretcher, spacer, and amino acid unit, can be synthesized by methods known in the art, such as those described in US2005-0238649A1.

Exemplary Drug Modules:

Maytansine and Maytansinoids

In some embodiments, the antibody-cytotoxic drug conjugate or pharmaceutically acceptable salt or solvate thereof comprises an antibody of the invention conjugated to one or more maytansinoid molecules. The maytansinoid is a mitotic inhibitor that acts by inhibiting tubulin multimerization. Maytansine was originally isolated from the Maytansine tree (Maytenus serrata) from the East African shrubs (U.S. Pat. No. 3,896,111). It was subsequently found that certain microorganisms also generate maytansinoids such as maytansinol and C-3 maytansinol ester (U.S. Pat. No. 4,151,042).

The maytansinoid drug modules are attractive drug modules in antibody-drug conjugates because they are: (i) relatively easy to be prepared from fermentation, or prepared by modification or derivation from the fermentation products; (ii) readily derived with a functional group suitable for coupling to an antibody through a non-disulfide linker; (iii) stable in plasma; and (iv) effective for variety of tumor cell lines.

Maytansine compounds suitable for use as the maytansinoid drug modules are well known in the art and can be isolated from natural sources according to known methods or produced using genetic engineering techniques (See Yu et al (2002) PNAS 99: 7968-7973). The maytansinol and maytansinol analogs can also be prepared according to known methods.

Exemplary embodiments of the maytansinoid drug module include: DM1, DM3 and DM4, as disclosed herein.

In some embodiments, the antibody-cytotoxic drug conjugate or pharmaceutically acceptable salt or solvate thereof comprises an antibody of the invention conjugated to dolastatin or dolastatin peptide analog or derivative (e.g., auristatin) (U.S. Pat. Nos. 5,635,483; 5,780,588). Dolastatin and auristatin have been shown to interfere with microtubule kinetics, GTP hydrolysis, and nuclear and cell division (Woyke et al. (2001) Antimicrob. Agents and Chemother. 45 (12): 3580-3584), and to have anti-cancer activity (U.S. Pat. No. 5,663,149) and antifungal activity (Pettit et al. (1998) Antimicrob. Agents Chemother. 42: 2961-2965). Dolastatin or auristatin drug modules may be attached to the antibody via the N (amino) terminus or the C (carboxy) terminus of the peptide drug module (WO002/088172).

Exemplary embodiments of auristatin include N-terminus linked monomethyl auristatin drug modules DE and DF, which are disclosed by Senter et al, Proceedings of the American Association for Cancer Research, volume 45, abstract number 623, Mar. 28, 2004, the disclosure of which is expressly incorporated herein by reference in its entirety. The peptide drug module may be selected from the general formulas D_{E} and D_{F} as below:

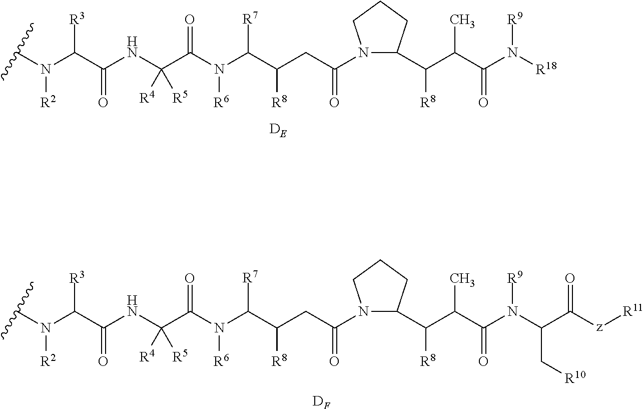

wherein the wavy lines of the D_{E} and D_{F} indicate the covalent attachment sites of the antibody or antibody-linker, and each site is independent from one another:

R² is selected from H and C1-C8 hydrocarbyl;

R³ is selected from the group consisting of H, C1-C8 hydrocarbyl, C3-C8 carbocycle, aryl, C1-C8 hydrocarbyl-aryl, C1-C8 hydrocarbyl-(C3-C8 carbocycle), C3-C8 heterocycle and C1-C8 hydrocarbyl —(C3-C8 heterocycle);

R⁴ is selected from the group consisting of H, C1-C8 hydrocarbyl, C3-C8 carbocycle, aryl, C1-C8 hydrocarbyl-aryl, C1-C8 hydrocarbyl-(C3-C8 carbocycle), C3-C8 heterocycle and C1-C8 hydrocarbyl-(C3-C8 heterocycle);

R⁵ is selected from H and methyl;

or R⁴ and R⁵ form a carbocycle of formula —(CRaRb)n-, wherein R^{a} and R^{b} are each independently selected from the group consisting of H, C1-C8 hydrocarbyl and C3-C8 carbocycle, and n is selected from 2, 3, 4, 5 and 6;

R⁶ is selected from H and C1-C8 hydrocarbyl;

R⁷ is selected from the group consisting of H, C1-C8 hydrocarbyl, C3-C8 carbocycle, aryl, C1-C8 hydrocarbyl-aryl, C1-C8 hydrocarbyl-(C3-C8 carbocycle), C3-C8 heterocycle and C1-C8 hydrocarbyl-(C3-C8 heterocycle);

Each R⁸ is independently selected from the group consisting of H, OH, C1-C8 hydrocarbyl, C3-C8 carbocycle and O—(C1-C8 hydrocarbyl);

R⁹ is selected from H and C1-C8 hydrocarbyl;

R¹⁰ is selected from aryl and C3-C8 heterocycle;

Z is selected from O, S, NH and NR¹², wherein R¹² is C1-C8 hydrocarbyl;

R¹¹ is selected from the group consisting of H, C1-C20 hydrocarbyl, aryl, C3-C8 heterocycle, —(R¹³O)m-R¹⁴ and —(R¹³O)m-CH(R¹⁵)₂;

m is an integer selected from 1-1000;

R¹³ is C₂-C8 hydrocarbyl;

R¹⁴ is H or C1-C8 hydrocarbyl;

R¹⁵ is independently selected from the group consisting of H, COOH, —(CH₂)ₙ—N(R₁₆)₂, —(CH₂)ₙ—SO₃H and —(CH₂)ₙ—SO₃-C1-C8 hydrocarbyl;

R¹⁶ is independently selected from the group consisting of H, C1-C8 hydrocarbyl and —(CH₂)ₙ—COOH;

R¹⁸ is selected from the group consisting of —C(R₈)₂—C(R₈)₂-aryl, —C(R₈)₂—C(R₈)₂—(C3-C8 heterocycle) and —C(R₈)₂—C(R₈)₂—(C3-C8 carbocycle); and

n is an integer selected from 0 to 6.

MMAE is an exemplary auristatin of the formula D_{E}, wherein the wavy line indicates a linker (L) covalently attached to the antibody-drug conjugate:

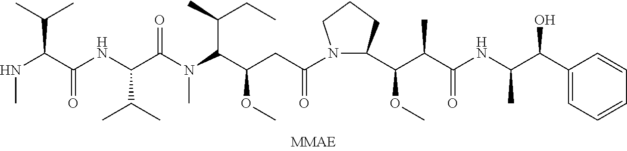

MMAF is an exemplary auristatin of the formula D_{F}, wherein the wavy line indicates a linker (L) covalently attached to the antibody-drug conjugate (see US2005/0238649 and Doronina et al (2006) Bioconjugate Chem. 17: 114-124):

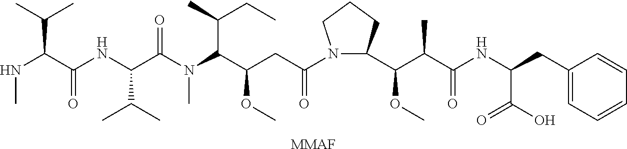

The other drug modules comprise MMAF derivative selected from the following, wherein the wavy line indicates a linker (L) covalently attached to the antibody-drug conjugate:

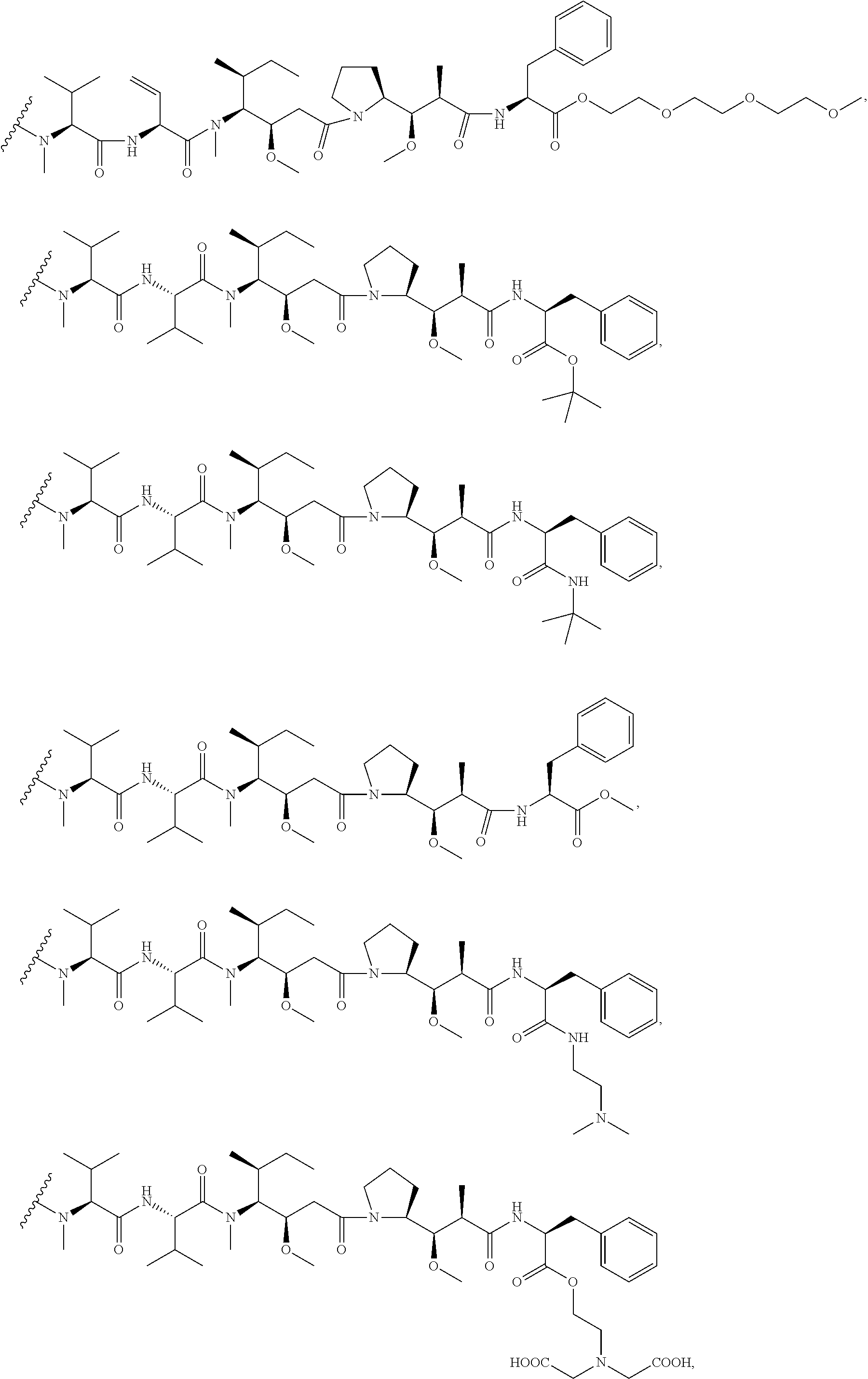

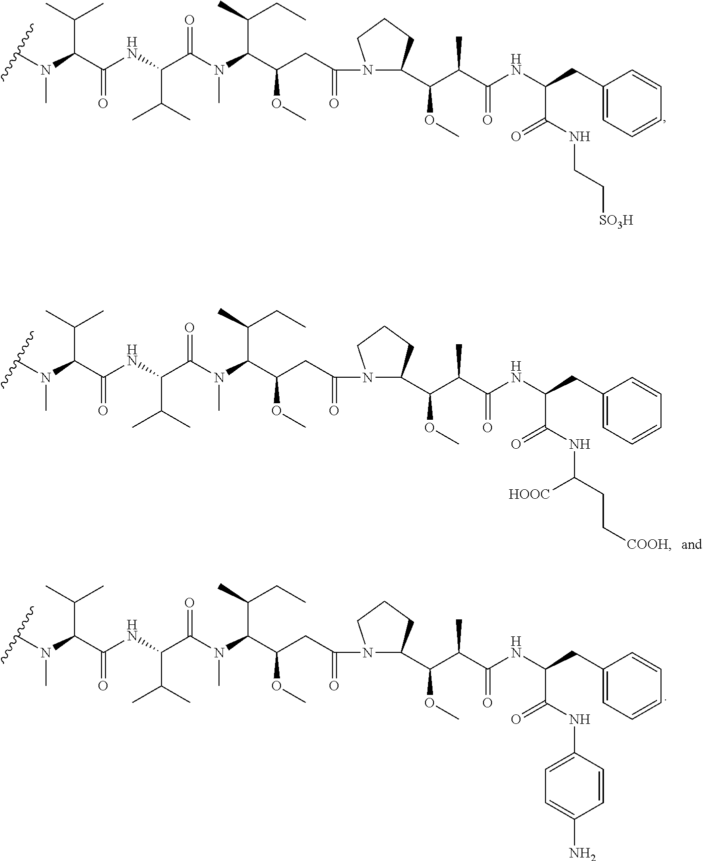 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NCCS(=O)(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)NC(CCC(=O)O)C(=O)O)OC)C(C)C)C(C)C ; *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)Nc1ccc(N)cc1)OC)C(C)C)C(C)C]

In one aspect, a hydrophilic group may be attached to a drug module at R¹¹, wherein said hydrophilic group includes, but is not limited to, triethylene glycol ester (TEG), as described above. Without being limited to any particular theory, the hydrophilic groups contribute to the internalization and non-agglomeration of the drug modules. Exemplary embodiments of ADC of general formula I comprising auristatin/dolastatin or a derivative thereof are described in US2005-0238649A1 and Doronina et al (2006) Bioconjugate Chem. 17:114-124, which is expressly incorporated herein by reference. Exemplary embodiments of ADCs of general formula I comprising MMAE or MMAF and various linkers have the following structure and abbreviations (wherein “Ab” is antibody; p ranges from 1 to about 8; “Val-Cit” is Valine-citrulline dipeptide; and “S” is a sulfur atom):

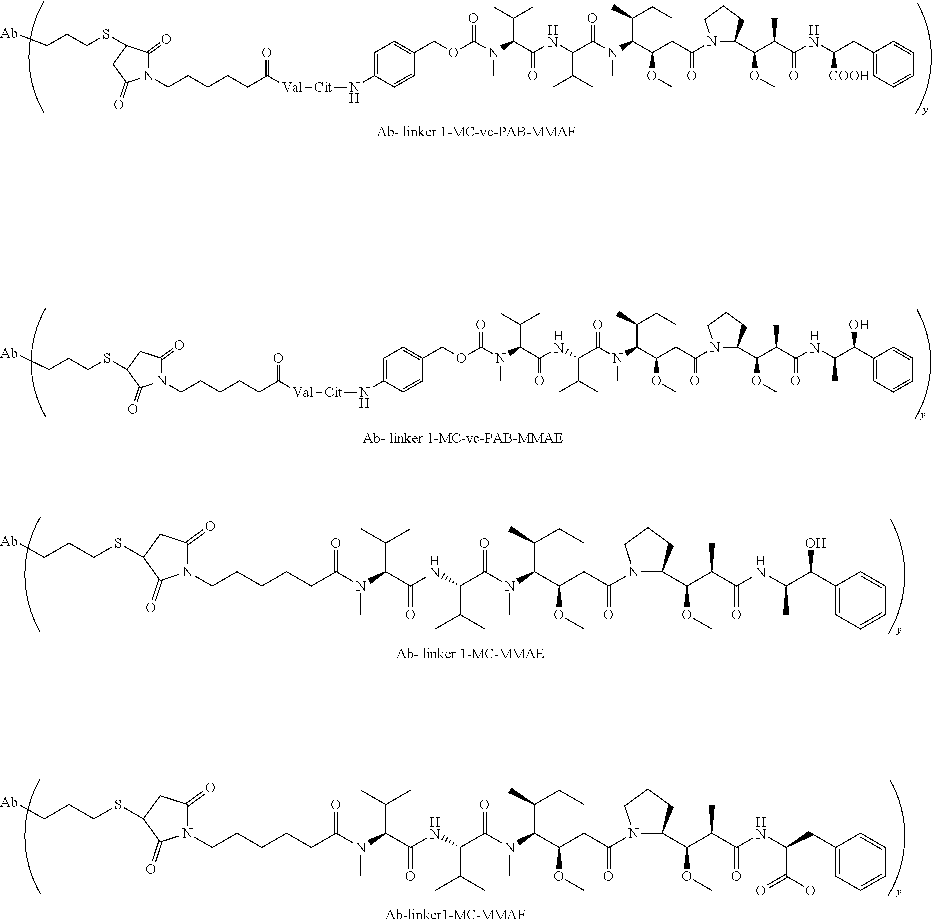

Typically, peptide-based drug modules can be prepared by forming peptide bonds between two or more amino acids and/or peptide fragments. Such peptide bonds can be prepared according to, for example, liquid phase synthesis methods well known in the art of peptide chemistry (see E. Schroder and K. Lubke, The Peptides, volume 1, pp 76-136, 1965, Academic Press). The auristatin/dolastatin drug modules can be prepared according to the methods described in the following literature: US20050238649A1; U.S. Pat. Nos. 5,635,483; 5,780,588; Pettit et al (1989) J. Am. Chem. Soc. 111: 5463-5465; Pettit et al (1998) Anti-Cancer Drug Design 13: 243-277; Pettit, G. R. et al, Synthesis, 1996, 719-725; Pettit et al (1996) J. Chem. Soc. Perkin Trans. 15: 859-863; and Doronina (2003) Nat. Biotechnol. 21(7): 778-784.

In particular, the auristatin/dolastatin drug modules of the general formula DF, such as MMAF and derivatives thereof, can be prepared using the methods described in US20050238649A1 and Doronina et al. (2006) Bioconjugate Chem. 17: 114-124. The auristatin/dolastatin drug modules of the general formula DE, such as MMAE and derivatives thereof, can be prepared by the method described in Doronina et al. (2003) Nat. Biotech. 21: 778-784. The drug-linker modules of MC-MMAF, MC-MMAE, MC-vc-PAB-MMAF and MC-vc-PAB-MMAE can be conveniently synthesized by conventional methods such as those described in Doronina et al. (2003) Nat. Biotech. 21: 778-784 and U.S. Patent Application Publication No. US2005/0238649A1, and then conjugated to the antibody of interest.

The Immunohistochemistry (IHC) of the present invention is used for detecting overexpression of c-Met protein in c-Met-positive tumor cells, and the expression amount is reflected based on the staining of cancer cells. Gray density analysis was performed with reference to perception test scoring or by using software.

The expression of c-Met is divided into two types: high expression and low expression. 3+(≥50% of tumor cells are strongly positive), 2+(≥50% of tumor cells are positive/weak positive, and <50% of tumor cells are strongly positive), 1+(≥50% of tumor cells are weakly positive, and <50% of cells are positive), 0 (no staining or tumor cells with any intensity <50%), and 2+ or 3+ is defined as high expression.

The c-Met positive marker scoring criteria are as follows:

(1) According to the degree of cell positive staining (antigen content), c-Met expression is divided into: weak positive (+), 1 score; medium positive (++), 2 score; or strongly positive (+++), 3 score;

(2) According to the number of positive cells, c-Met expression is divided into: weakly positive (+, refers to the total number of positive cells below 25%), medium positive (++, refers to the total number of positive cells between 25%-49%), strongly positive (+++, refers to the total number of positive cells above 50%).

At present, weighted integral measurement is used. The calculation formula is: (+)%×1+(++)%×2+(+++)%×3; a score of 0-0.3 is weakly positive, 0.3-1.5 is medium positive, and 1.5-3 is strongly positive.

The c-Met positive or overexpression of c-Met described in the present invention means that ≥20% of the hepatic carcinoma cells are positive/weakly positive, preferably ≥25% of the hepatic carcinoma cells are positive, more preferably ≥50% of the hepatic carcinoma cells are strongly positive.

Drug Load

The drug load is represented by y, and is the mean number of drug modules per antibody in the molecule of formula I. The drug load can range from 1 to 20 drug modules (D) per antibody. The ADC of Formula I includes a collection of antibodies conjugated to a range of (1-20) drug modules. The mean number of drug modules per antibody in an ADC preparation obtained from a coupling reaction can be characterized by conventional means such as mass spectrometry, ELISA assay, and HPLC. It is also possible to determine the quantitative distribution of ADCs with respect to y. In some cases, homogeneous ADCs with certain p values are isolated from ADCs of other drug loads, and then purified and characterized. This can be achieved by means such as reverse phase HPLC or electrophoresis.

For some antibody-drug conjugates, y may be limited by the number of attachment sites on the antibody. For example, if the cysteine thiol is attached, as in the above illustrative embodiment, the antibody may have only one or several cysteine thiol groups, or may have only one or more reactive thiol groups which can be attached to the linker. In certain embodiments, a higher drug loading, such as y>5, may cause aggregation, insolubility, toxicity, or loss of cell permeability of certain antibody-drug conjugates. In certain embodiments, the drug loading of the ADC of the invention ranges from 1 to about 8; from about 2 to about 6; from about 3 to about 5; from about 3 to about 4; from about 3.1 to about 3.9; from about 3.2 to about 3.8; about 3.2 to about 3.7; about 3.2 to about 3.6; about 3.3 to about 3.8; or about 3.3 to about 3.7. In fact, for some ADCs, it has been shown that the optimal ratio of each drug module to antibody may be less than 8 and may be from about 2 to about 5. See US20050238649A1 (incorporated herein by reference in its entirety).

In certain embodiments, less than the theoretical maximum number of drug molecules are coupled to the antibody in the coupling reaction. The antibody may comprise, for example, a lysine residue that does not react with a drug-linker intermediate or linker agent, as discussed below. Only the most reactive lysine groups can react with amine-reactive linker agents. In general, the antibody does not contain a number of free and reactive cysteine thiol groups, which can be linked to a drug module; in fact, most of the cysteine thiol groups in the antibody are present in the form of a disulfide bridge. In certain embodiments, the antibody may be reduced with a reducing agent such as dithiothreitol (DTT) or tricarbonyl ethyl phosphine (TCEP) under partially or completely reductive conditions to produce a reactive cysteine thiol group. In certain embodiments, the antibody is placed under denaturing conditions to expose a reactive nucleophilic group, such as lysine or cysteine.

The drug load (drug/antibody ratio, DAR) of the ADC can be controlled in different ways, for example by: (i) limiting the molar excess of the drug-linker intermediate or linker agent; (ii) limiting the time or temperature of the coupling reaction; (iii) limiting the thiol modification of the cysteine or restricting the reductive condition; (iv) engineering the amino acid sequence of the antibody by recombinant techniques, such that the number and location of cysteine residues are altered in order to control the number and/or position of the linker-drug attachment (such as the thioMab or the thioFab prepared as those described in the present invention and WO2006/034488 (incorporated herein by reference in its entirety)).

It is to be understood that if more than one nucleophilic group is reacted with a drug-linker intermediate or with a linker and subsequent drug module agents, the resulting product is an ADC compound mixture having one or more drug modules attached to the antibody. The mean number of drugs per antibody can be calculated from the mixture by ELISA assay which involves antibody-specific and drug-specific antibodies. The various ADC molecules in the mixture can be identified by mass spectrometry, and separated by HPLC, for example, by hydrophobic interaction chromatography. In certain embodiments, a homogeneous ADC with a single load value can be isolated from the coupling mixture by electrophoresis or chromatography.

Methods for Preparing Antibody-Cytotoxic Drug Conjugates or Pharmaceutically Acceptable Salts or Solvates Thereof

The ADC of general formula I can be prepared by several routes using organic chemical reactions, conditions and agents known to those skilled in the art, including: (1) the nucleophilic group of the antibody reacts with the divalent linker agent via a covalent bond to form Ab-L, followed by reaction with the drug module D; and (2) the nucleophilic group of the drug module reacts with the divalent linker agent via a covalent bond to form D-L, followed by reaction with the nucleophilic group of the antibody. The exemplary method for preparing the ADC of Formula I via the latter route is described in US2005-0238649A1, which is expressly incorporated herein by reference.

Nucleophilic groups of antibodies include, but are not limited to: (i) an N-terminal amine group; (ii) a side chain amine group such as lysine; (iii) a side chain thiol group such as cysteine; and (iv) a hydroxyl or amino group of saccharide in the glycosylated antibody. Amines, thiols and hydroxyl groups are nucleophilic and are capable of reacting with the electrophilic groups on the linker module to form covalent bonds, and the linker agents include: (i) active esters such as NHS esters, HOBt esters, haloformates, and acid halides; (ii) hydrocarbyl and benzyl halides, such as haloacetamides; (iii) aldehydes, ketones, carboxyl groups and maleimide groups. Some antibodies have a reducible interchain disulfide, that is, a cysteine bridge. The antibody can be completely or partially reduced by treatment with a reducing agent such as DTT (dithiothreitol) or tricarbonyl ethylphosphine (TCEP) to provide coupling reactivity with the linker. Each cysteine bridge will theoretically form two reactive thiol nucleophiles. Alternatively, the sulfhydryl group may be introduced into the antibody via modification of the lysine residue, for example by reacting the lysine residue with 2-iminothiolane (Traut reagent), resulting in the conversion of the amine to the thiol.

The antibody-drug conjugates of the present invention can also be produced by the reaction between an electrophilic group on an antibody (such as an aldehyde or ketone carbonyl group) and a linker or nucleophilic group on a drug. Useful nucleophilic groups on the linker include, but are not limited to: hydrazide, oxime, amino, hydrazine, thiosemicarbazone, hydrazine carboxylate and arylhydrazide. In one embodiment, the saccharide of the glycosylated antibody can be oxidized with, for example, a periodate oxidant to form an aldehyde or ketone group that can react with the amine group of the linker or drug module. The resulting imine Schiff base may form a stable linkage or may be reduced with, for example, a borohydride agent to form a stable amine linkage. In one embodiment, the reaction of the carbohydrate moiety of the glycosylated antibody with galactose oxidase or sodium metaperiodate may produce a carbonyl group (aldehyde group and keto group) in the antibody, which may be reacted with a suitable group on the drug (Hermanson, Bioconjugate Techniques). In another embodiment, an antibody comprising an N-terminal serine or threonine residue may react with sodium metaperiodate, resulting in the formation of an aldehyde at the first amino acid (Geoghegan and Stroh, (1992) Bioconjugate Chem. 3: 138-146; U.S. Pat. No. 5,362,852). Such aldehydes can react with the drug module or the linker nucleophile.

Nucleophilic groups on the drug module include, but are not limited to: amine, thiol, hydroxy, hydrazide, oxime, hydrazine, thiosemicarbazone, hydrazine carboxylate and arylhydrazide groups, which can react with the electrophilic groups on the linker module to form covalent bonds. The linker agents include: (i) active esters such as NHS esters, HOBt esters, haloformates, and acid halides; (ii) hydrocarbyl and benzyl halides, such as haloacetamides; and (iii) aldehydes, ketones, carboxyl groups, and maleimide groups.

The compounds of the present invention clearly cover but are not limited to the ADC prepared by the following crosslinking agents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB and SVSB (succinimidyl-(4-vinylsulfone) benzoate), which are commercially available (such as Pierce Biotechnology, Inc., Rockford, Ill., U.S.A, refer to the 2003-2004 Application Manual and product catalog (2003-2004 Applications Handbook and Catalog) pages 467-498).

Antibody-cytotoxic drug conjugates or their pharmaceutically acceptable salts or solvates containing antibodies and cytotoxic agents can also be prepared using a variety of bifunctional protein coupling agents, such as N-succinimidyl 3-(2-pyridyldithio) propionate (SPDP), succinimidyl-4-(N-maleimidomethyl) cyclohexane-1-carboxylate (SMCC), aminosulfane (IT), imidates (such as dimethyl adipamide HCl), active esters (such as disuccinimidyl suberate), aldehydes (such as glutaraldehyde), bis azide compounds (such as bis (p-azidobenzoyl) hexamethylene diamine), bis diazo derivatives (such as bis (p-diazo benzoyl)-ethylenediamine), diisothiocyanate (such as toluene 2,6-Diisocyanate) and dual active fluorine compounds (such as 1,5-difluoro-2,4-dinitrobenzene). For example, ricin immunotoxins can be prepared as described in Vitetta et al., Science 238: 1098 (1987). The carbon-14 labeled 1-isothiocyanate benzyl-3-methyl diethylene triamine pentaacetic acid (MX-DTPA) is an exemplary chelating agent for coupling a radioactive nucleotide to an antibody. See WO 94/11026.

Alternatively, a fusion protein comprising an antibody and a cytotoxic agent can be prepared by, for example, recombinant techniques or peptide synthesis. The recombinant DNA molecule may comprise regions encoding the antibody and cytotoxic moiety of the conjugate respectively, either adjacent to each other or separated by a region encoding a linker peptide, wherein said linker peptide does not destroy the desired properties of the conjugate.

In yet another embodiment, the antibody may be conjugated to a “receptor”; (such as streptavidin) for pre-targeting the tumor, the antibody-receptor conjugate is administered to a patient, followed by the use of a scavenger which removes the unbound conjugates from circulation. Then, a “ligand” (e.g., avidin) coupled to a cytotoxic agent (such as a radioactive nucleotide) is administrated. The following examples are provided for illustrative purposes only and are not intended to limit the scope of the invention.

### Beneficial Effects of the Invention

When compared with the prior art, the technical solution of the present invention has the following advantages:

(1) The anti-c-Met antibody-cytotoxic drug conjugate (ADC) of the present invention, alone, has a significant inhibitory effect on hepatic carcinoma cells, effectively inhibits the proliferation thereof, and can inhibit the growth and volume of hepatic carcinoma cells for a long time. On the other hand, the antibody alone has a poor inhibitory effect on hepatic carcinoma cells, and the difference between the antibody and the ADC is remarkable. Therefore, the ADC of the present invention can be preferably used as a pharmaceutically active ingredient.

(2) Studies have shown that the anti-c-Met antibody-cytotoxic drug conjugate (ADC) of the present invention is well tolerated in animals, and the anti-c-Met ADC obtained by the technical solution of the present invention can be applied to industrial production.

### BRIEF DESCRIPTION OF THE DRAWINGS

FIG. 1 is the curve tracing the tumor volume, showing the inhibition of hepatic carcinoma cells by ADC molecules of the present invention in a LI-03-0022 HCC PDX tumor model. The results show that the ADC molecule can achieve complete tumor inhibition by the introduced toxin, whereas the antibody alone cannot. The ADC drug of the invention has good tolerance in tumor-bearing animals; the data represent the mean value of a group, and the error bars represent the standard error of the mean value (SEM);

FIG. 2 is the IHC (immunohistochemical staining) score of LI-03-0010 (negative control) in a LI-03-0022 HCC PDX tumor model;

FIG. 3 is the IHC (immunohistochemical staining) score of LI-03-0022 tissue in a LI-03-0022 HCC PDX tumor model;

FIG. 4 is the curve tracing the tumor volume, showing the inhibition of hepatic carcinoma cells by ADC molecules of the present invention in a LI-03-0240 HCC PDX tumor model;

FIG. 5 is the IHC (immunohistochemical staining) score of LI-03-0010 (negative control) in a LI-03-0240 HCC PDX tumor model;

FIG. 6 is the IHC (immunohistochemical staining) score of LI-03-0240 tissue in a LI-03-0240 HCC PDX tumor model.

### DETAILED DESCRIPTION OF THE INVENTION

Hereinafter, the present invention is illustrated in more details with reference to examples. The examples of the invention are merely used to exemplify the technical solution of the invention, and the merits and scope of the invention are not limited thereto.

In the examples or test examples of the present invention, where specific conditions are not described, the experiments are generally conducted under conventional conditions, or under conditions proposed by the manufacturers of the material or product. See Sambrook et al., Molecular Cloning, A Laboratory Manual, Cold Spring Harbor Laboratory; Current Protocols in Molecular Biology, Ausubel et al, Greene Publishing Associates, Wiley Interscience, NY. Where the source of the agents is not specifically given, the agents are commercially available.

### EXAMPLES

### Example 1. Clonal Expression of Antigen and Antibody

The antibodies (light and heavy chains) and antigens used in the present invention are constructed by overlapping extension PCR methods known in the art. The DNA fragment obtained by overlapping extension PCR was inserted into the expression vector pEE6.4 (Lonza Biologics) using HindIII/BstBI restriction sites, and expressed in 293F cells (Invitrogen, Cat # R790-07). The resulting recombinant protein was used for immunization or screening. The c-Met gene template was derived from origene Corporation (number RC217003). The DNA sequences cloned and expressed are as follows:

DNA sequence for fusion protein of human c-Met extracellular region (ECD) and murine Fc region (human c-Met ECD-mFc):

(SEQ ID NO: 1)
atgaaggcccccgctgtgcttgcacctggcatcctcgtgctcctgtttac
cttggtgcagaggagcaatggggagtgtaaagaggcactagcaaagtccg
agatgaatgtgaatatgaagtatcagcttcccaacttcaccgcggaaaca
cccatccagaatgtcattctacatgagcatcacattttccttggtgccac
taactacatttatgttttaaatgaggaagaccttcagaaggttgctgagt
acaagactgggcctgtgctggaacacccagattgtttcccatgtcaggac
tgcagcagcaaagccaatttatcaggaggtgtttggaaagataacatcaa
catggctctagttgtcgacacctactatgatgatcaactcattagctgtg
gcagcgtcaacagagggacctgccagcgacatgtctttccccacaatcat
actgctgacatacagtcggaggttcactgcatattctccccacagataga
agagcccagccagtgtcctgactgtgtggtgagcgccctgggagccaaag
tcctttcatctgtaaaggaccggttcatcaacttctttgtaggcaatacc
ataaattcttcttatttcccagatcatccattgcattcgatatcagtgag
aaggctaaaggaaacgaaagatggttttatgtttttgacggaccagtcct
acattgatgttttacctgagttcagagattcttaccccattaagtatgtc
catgcctttgaaagcaacaattttatttacttcttgacggtccaaaggga
aactctagatgctcagacttttcacacaagaataatcaggttctgttcca
taaactctggattgcattcctacatggaaatgcctctggagtgtattctc
acagaaaagagaaaaaagagatccacaaagaaggaagtgtttaatatact
tcaggctgcgtatgtcagcaagcctggggcccagcttgctagacaaatag
gagccagcctgaatgatgacattcttttcggggtgttcgcacaaagcaag
ccagattctgccgaaccaatggatcgatctgccatgtgtgcattccctat
caaatatgtcaacgacttcttcaacaagatcgtcaacaaaaacaatgtga
gatgtctccagcatttttacggacccaatcatgagcactgctttaatagg
acacttctgagaaattcatcaggctgtgaagcgcgccgtgatgaatatcg
aacagagtttaccacagctttgcagcgcgttgacttattcatgggtcaat
tcagcgaagtcctcttaacatctatatccaccttcattaaaggagacctc
accatagctaatcttgggacatcagagggtcgcttcatgcaggttgtggt
ttctcgatcaggaccatcaacccctcatgtgaattttctcctggactccc
atccagtgtctccagaagtgattgtggagcatacattaaaccaaaatggc
tacacactggttatcactgggaagaagatcacgaagatcccattgaatgg
cttgggctgcagacatttccagtcctgcagtcaatgcctctctgccccac
cctttgttcagtgtggctggtgccacgacaaatgtgtgcgatcggaggaa
tgcctgagcgggacatggactcaacagatctgtctgcctgcaatctacaa
ggttttcccaaatagtgcaccccttgaaggagggacaaggctgaccatat
gtggctgggactttggatttcggaggaataataaatttgatttaaagaaa
actagagttctccttggaaatgagagctgcaccttgactttaagtgagag
cacgatgaatacattgaaatgcacagttggtcctgccatgaataagcatt
tcaatatgtccataattatttcaaatggccacgggacaacacaatacagt
acattctcctatgtggatcctgtaataacaagtatttcgccgaaatacgg
tcctatggctggtggcactttacttactttaactggaaattacctaaaca
gtgggaattctagacacatttcaattggtggaaaaacatgtactttaaaa
agtgtgtcaaacagtattcttgaatgttataccccagcccaaaccatttc
aactgagtttgctgttaaattgaaaattgacttagccaaccgagagacaa
gcatcttcagttaccgtgaagatcccattgtctatgaaattcatccaacc
aaatcttttattagtggtgggagcacaataacaggtgttgggaaaaacct
gaattcagttagtgtcccgagaatggtcataaatgtgcatgaagcaggaa
ggaactttacagtggcatgtcaacatcgctctaattcagagataatctgt
tgtaccactccttccctgcaacagctgaatctgcaactccccctgaaaac
caaagcctttttcatgttagatgggatcctttccaaatactttgatctca
tttatgtacataatcctgtgtttaagccttttgaaaagccagtgatgatc
tcaatgggcaatgaaaatgtactggaaattaagggaaatgatattgaccc
tgaagcagttaaaggtgaagtgttaaaagttggaaataagagctgtgaga
atatacacttacattctgaagccgttttatgcacggtccccaatgacctg
ctgaaattgaacagcgagctaaatatagagtggaagcaagcaatttcttc
aaccgtccttggaaaagtaatagttcaaccagatcagaatttcaca

DNA sequence for human c-Met extracellular Sema region and Flag-His tag (Human c-Met Sema-Flis):

(SEQ ID NO: 2)
atgaaggcccccgctgtgcttgcacctggcatcctcgtgctcctgtttac
cttggtgcagaggagcaatggggagtgtaaagaggcactagcaaagtccg
agatgaatgtgaatatgaagtatcagcttcccaacttcaccgcggaaaca
cccatccagaatgtcattctacatgagcatcacattttccttggtgccac
taactacatttatgttttaaatgaggaagaccttcagaaggttgctgagt
acaagactgggcctgtgctggaacacccagattgtttcccatgtcaggac
tgcagcagcaaagccaatttatcaggaggtgtttggaaagataacatcaa
catggctctagttgtcgacacctactatgatgatcaactcattagctgtg
gcagcgtcaacagagggacctgccagcgacatgtctttccccacaatcat
actgctgacatacagtcggaggttcactgcatattctccccacagataga
agagcccagccagtgtcctgactgtgtggtgagcgccctgggagccaaag
tcctttcatctgtaaaggaccggttcatcaacttctttgtaggcaatacc
ataaattcttcttatttcccagatcatccattgcattcgatatcagtgag
aaggctaaaggaaacgaaagatggttttatgtttttgacggaccagtcct
acattgatgttttacctgagttcagagattcttaccccattaagtatgtc
catgcctttgaaagcaacaattttatttacttcttgacggtccaaaggga
aactctagatgctcagacttttcacacaagaataatcaggttctgttcca
taaactctggattgcattcctacatggaaatgcctctggagtgtattctc
acagaaaagagaaaaaagagatccacaaagaaggaagtgtttaatatact
tcaggctgcgtatgtcagcaagcctggggcccagcttgctagacaaatag
gagccagcctgaatgatgacattcttttcggggtgttcgcacaaagcaag
ccagattctgccgaaccaatggatcgatctgccatgtgtgcattccctat
caaatatgtcaacgacttcttcaacaagatcgtcaacaaaaacaatgtga
gatgtctccagcatttttacggacccaatcatgagcactgctttaatagg
acacttctgagaaattcatcaggctgtgaagcgcgccgtgatgaatatcg
aacagagtttaccacagctttgcagcgcgttgacttattcatgggtcaat
tcagcgaagtcctcttaacatctatatccaccttcattaaaggagacctc
accatagctaatcttgggacatcagagggtcgcttcatgcaggttgtggt
ttctcgatcaggaccatcaacccctcatgtgaattttctcctggactccc
atccagtgtctccagaagtgattgtggagcatacattaaaccaaaatggc
tacacactggttatcactgggaagaagatcacgaagatcccattgaatgg
cttgggctgcagacatttccagtcctgcagtcaatgcctctctgccccac
cctttgttcagtgtggctggtgccacgacaaatgtgtgcgatcggaggaa
tgcctgagcgggacatggactcaacagatctgtctgcctgcaatctacaa
ggactacaaggacgacgacgacaagcatgtccaccatcatcaccatcact
gattcgaa

DNA sequence for human c-Met ECD his tag (Human c-Met ECD-His) recombinant protein:

(SEQ ID NO: 3)
atgaaggcccccgctgtgcttgcacctggcatcctcgtgctcctgtttac
cttggtgcagaggagcaatggggagtgtaaagaggcactagcaaagtccg
agatgaatgtgaatatgaagtatcagcttcccaacttcaccgcggaaaca
cccatccagaatgtcattctacatgagcatcacattttccttggtgccac
taactacatttatgttttaaatgaggaagaccttcagaaggttgctgagt
acaagactgggcctgtgctggaacacccagattgtttcccatgtcaggac
tgcagcagcaaagccaatttatcaggaggtgtttggaaagataacatcaa
catggctctagttgtcgacacctactatgatgatcaactcattagctgtg
gcagcgtcaacagagggacctgccagcgacatgtctttccccacaatcat
actgctgacatacagtcggaggttcactgcatattctccccacagataga
agagcccagccagtgtcctgactgtgtggtgagcgccctgggagccaaag
tcctttcatctgtaaaggaccggttcatcaacttctttgtaggcaatacc
ataaattcttcttatttcccagatcatccattgcattcgatatcagtgag
aaggctaaaggaaacgaaagatggttttatgtttttgacggaccagtcct
acattgatgttttacctgagttcagagattcttaccccattaagtatgtc
catgcctttgaaagcaacaattttatttacttcttgacggtccaaaggga
aactctagatgctcagacttttcacacaagaataatcaggttctgttcca
taaactctggattgcattcctacatggaaatgcctctggagtgtattctc
acagaaaagagaaaaaagagatccacaaagaaggaagtgtttaatatact
tcaggctgcgtatgtcagcaagcctggggcccagcttgctagacaaatag
gagccagcctgaatgatgacattcttttcggggtgttcgcacaaagcaag
ccagattctgccgaaccaatggatcgatctgccatgtgtgcattccctat
caaatatgtcaacgacttcttcaacaagatcgtcaacaaaaacaatgtga
gatgtctccagcatttttacggacccaatcatgagcactgctttaatagg
acacttctgagaaattcatcaggctgtgaagcgcgccgtgatgaatatcg
aacagagtttaccacagctttgcagcgcgttgacttattcatgggtcaat
tcagcgaagtcctcttaacatctatatccaccttcattaaaggagacctc
accatagctaatcttgggacatcagagggtcgcttcatgcaggttgtggt
ttctcgatcaggaccatcaacccctcatgtgaattttctcctggactccc
atccagtgtctccagaagtgattgtggagcatacattaaaccaaaatggc
tacacactggttatcactgggaagaagatcacgaagatcccattgaatgg
cttgggctgcagacatttccagtcctgcagtcaatgcctctctgccccac
cctttgttcagtgtggctggtgccacgacaaatgtgtgcgatcggaggaa
tgcctgagcgggacatggactcaacagatctgtctgcctgcaatctacaa
ggttttcccaaatagtgcaccccttgaaggagggacaaggctgaccatat
gtggctgggactttggatttcggaggaataataaatttgatttaaagaaa
actagagttctccttggaaatgagagctgcaccttgactttaagtgagag
cacgatgaatacattgaaatgcacagttggtcctgccatgaataagcatt
tcaatatgtccataattatttcaaatggccacgggacaacacaatacagt
acattctcctatgtggatcctgtaataacaagtatttcgccgaaatacgg
tcctatggctggtggcactttacttactttaactggaaattacctaaaca
gtgggaattctagacacatttcaattggtggaaaaacatgtactttaaaa
agtgtgtcaaacagtattcttgaatgttataccccagcccaaaccatttc
aactgagtttgctgttaaattgaaaattgacttagccaaccgagagacaa
gcatcttcagttaccgtgaagatcccattgtctatgaaattcatccaacc
aaatcttttattagtggtgggagcacaataacaggtgttgggaaaaacct
gaattcagttagtgtcccgagaatggtcataaatgtgcatgaagcaggaa
ggaactttacagtggcatgtcaacatcgctctaattcagagataatctgt
tgtaccactccttccctgcaacagctgaatctgcaactccccctgaaaac
caaagcctttttcatgttagatgggatcctttccaaatactttgatctca
tttatgtacataatcctgtgtttaagccttttgaaaagccagtgatgatc
tcaatgggcaatgaaaatgtactggaaattaagggaaatgatattgaccc
tgaagcagttaaaggtgaagtgttaaaagttggaaataagagctgtgaga
atatacacttacattctgaagccgttttatgcacggtccccaatgacctg
ctgaaattgaacagcgagctaaatatagagtggaagcaagcaatttcttc
aaccgtccttggaaaagtaatagttcaaccagatcagaatttcacacacc
atcatcaccatcactgattcgaa

### Example 2. Binding Assay of Antibody and Antigen (ELISA)

This experiment uses enzyme linked immunosorbent assay to detect affinity of c-Met antibody (including supermatant of hybridonma or recombinant expressed monoclonal antibodies) to c-Met antigen in vitro.

Experimental procedures: Coating buffer (PBS; Hyclone, Cat No.: SH30256.01B) was used to dilute antigen (human c-Met-His, example 1) to 2 μg/mL, which was added to a 96-well microplate at 100 μL/well (Costar 9018, Cat No.:03113024) and incubated overnight at 4° C. The next day, the antibody-coated 96-well microplate was restored to room temperature and was washed three times with washing buffer (PBS+0.05% Tween 20 (Sigma, Cat No.:P1379). Blocking buffer was added at 200 μL/well (PBS+1% BSA (Roche, Cat No.:738328) and the plate was incubated at 37° C. for 1 hour. The plate was then washed three times with washing buffer. The anti c-Met antibody to be tested was added to the 96-well microplate and was incubated for 1 hour at room temperature. The plate was then washed three times with washing buffer. Secondary antibody (Goat anti-Mouse IgG(H+L)(HRP) (Thermo, No.:31432) diluted with blocking buffer (10000× dilution) was added to the 96-well microplate at 100 μL/well and the plate was incubated for 1 hour at room temperature. The plate was then washed three times and TMB chromogenic substrate (eBioscience REF:00-4201-56) was added to the 96-well microplate at 100 μL/well. Stop solution 2N H₂SO₄ was added to the 96-well microplate at 100 μL/well. The plate was read with plate reader at 450 nm.

### Example 3. Production of Murine Monoclonal Antibody Cell Strain Against Human c-Met

Murine anti-human c-Met monoclonal cell lines were obtained by immunizing mice, fusion of spleen cells, and screening of hybridomas. This method is well-known in this field. Recombinant expressed antigen (human c-Met ECD-mFc, human c-Met Sema-flis, see example 1) was diluted to 1 mg/mL with PBS (Hyclone, Cat No.:SH30256.01B) and emulsified with Freund's adjuvant (the first immunization was performed with Freund's complete adjuvant, and the booster immunizations were performed with Freund's incomplete adjuvant); and injected into Balb/C mice subcutaneously (5 mice/group) with each mouse inoculated with 100 μg antigen, and booster immunizations were given every two weeks. After the first booster immunization, mice serum was collected during 7 to 10 days after each booster immunization, and the titer was detected by ELISA (Methods were described in Example 2).

After immunization, mice with a serum titer higher than 1:10⁵ were selected for cell fusion. Mouse B-cells and myeloma cells (SP2/0, ATCC number:CRL-1581™) were prepared respectively in aseptic conditions and counted. The two kinds of cells were mixed at a ratio of B-cells:SP2/0 of 1:4 and then were centrifuged (1500 r/min, 7 min). The supernatant was discarded and 1 mL of 50% polyethylene glycol (Supplier: SIGMA, Catalogue # RNBB306) was added. Next, 1 mL serum-free RPMI1640 (Supplier: GIBCO, Catalogue # C22400) was used for termination, and samples were centrifuged for 10 minutes. The supernatant was then discarded. The pellet was resuspended in RPMI1640 which comprised hybridoma cell growth factor (Supplier: Roche, Catalogue #1363735001), serum (Supplier: GIBCO, Catalogue # C20270) and HAT (Supplier: Invitrogen, Catalogue #21060-017). B-cells were plated on the plate at 10⁵ cells/well and each well was 100 μL. The plate was placed in a cell incubator at 37° C. Three days later, 100 μL of RPMI1640, which comprised hybridoma cell growth factor, serum and HT (Supplier: Invitrogen, Catalogue #11067-030) was added to each well. After 2 to 4 days, each well was replaced with 150 L RPMI1640 comprising hybridoma cell growth factor, serum and HT. The next day, positive clones were detected by ELISA (see Methods in Example 2).

Experimental Results:

TABLE 1
Detection of hybridoma fusion of mice immunized with human c-Met

| Clone No. | Detection Results (OD450) |
|---|---|
| Negative control | 0.07 |
| Ab-1 | 1.48 |
| Ab-2 | 1.38 |
| Ab-3 | 1.29 |
| Ab-4 | 1.6 |
| Ab-5 | 1.64 |
| Ab-6 | 1.75 |
| Ab-7 | 1.58 |
| Ab-8 | 1.24 |

### Example 4. Cloning of Anti c-Met Antibody Sequence

The cell line Ab-5 was selected for cDNA sequence cloning. The mAb was recombinantly expressed and subjected to various activity tests. The variable regions of the heavy chain and light chain of the antibody gene were amplified by reverse transcription PCR, and ligated to vector to obtain the heavy and light chain sequences of the monoclonal antibody by sequencing. First, an RNA purification kit (Qiagen company, Cat. No. 74134, see the instructions for this procedure) was used to extract total RNAs from the active single cell stain from example 3. Next, single stranded cDNA was prepared by the cDNA synthesis kit (Invitrogen company, Cat. No. 18080-051), which involves cDNA reverse transcription using Oligo-dT primers. The product served as a template, and the variable region sequence of the antibody heavy and light chain was synthesized by PCR. The products of PCR were cloned into the TA vector pMD-18T and then sequenced. The obtained heavy and light chain sequences of the antibody were separately cloned to expression vectors (see example 1), and the recombinant monoclonal antibody was expressed to prove its activity (see examples 2 and 3), followed by humanization.

Sequence of mouse hybridoma cell monoclonal
antibody Ab-5:
Heavy chain variable region:
(SEQ ID NO: 4)
QVQLKQSGPGLVQPSQSLSITCTVSGFSLPNYGVHWVRQSPGKGLEWLGV
IWSGGSTNYAAAFVSRLRISKDNSKSQVFFEMNSLQADDTAVYYCARNHD
NPYNYAMDYWGQGTTVTVSS
Light chain variable region:
(SEQ ID NO: 5)
DIVLTQSPGSLAVYLGQRATISCRANKSVSTSTYNYLHWYQQKPGQPPKL
LIYLASNLASGVPARFSGSGSGTDFTLNIHPLEEEDAATYYCQHSRDLPP
TFGAGTKLELKR

The amino acid residues of VH/VL CDR of anti-human c-Met antibodies were determined and annotated by the Kabat numbering system.

CDR sequences of murine in the invention are shown in Table below:

TABLE 2
CDR sequence of Murine anti-sclerostin antibody

| Antibody | Ab-5 | |
|---|---|---|
| Heavy chain CDR1 | NYGVH | (SEQ ID NO: 6) |
| Heavy chain CDR2 | VIWSGGSTNYAAAFVS | (SEQ ID NO: 7) |
| Heavy chain CDR3 | NHDNPYNYAMDY | (SEQ ID NO: 8) |
| Light chain CDR1 | RANKSVSTSTYNYLH | (SEQ ID NO: 9) |
| Light chain CDR2 | LASNLAS | (SEQ ID NO: 10) |
| Light chain CDR3 | QHSRDLPPT | (SEQ ID NO: 11) |

### Example 5. Humanization of Anti c-Met Antibody

The murine anti c-Met monoclonal antibody heavy and light chain sequences obtained from example 4 were aligned against an antibody database for homology, and a humanized antibody model was established then. The optimal humanized c-Met monoclonal antibody was selected as the preferred molecule of the invention according to the model for back-mutation. A crystal structure showing similar homology with the obtained murine candidate molecules was selected from the published database of mice Fab crystal structure models (e.g. PDB database), and a Fab crystal structure with high resolution (such as, less than 2.5 Å) was selected; and the mouse Fab model was established. The murine antibody heavy and light chain sequences of the invention were aligned against the sequences in the model, and the constant sequence was maintained so that the structural model of the mouse antibody of the invention could be obtained. The variable amino acids might be potential sites for back-mutation. Swiss-pdb viewer software was used to run the mouse antibody structure model to optimize energy (minimization). Back-mutation was performed at different amino acid sites other than those in CDRs of the model, and the activities of the resultant humanized antibody and of the antibody without humanization were compared. A humanized antibody with good activity was maintained. The CDR region was further optimized, including mutations to prevent glycosylation, deamination, oxidation sites and so on. CDR regions of the optimized humanized anti c-Met antibody are shown in table below:

TABLE 3
CDR sequence of the optimized anti c-Met antibody

| Antibody | Optimized humanized antibody | |
|---|---|---|
| Heavy Chain CDR1 | NYGVH | (SEQ ID NO: 6) |
| Heavy Chain CDR2 | VIWSGGSTNYAAAFVS | (SEQ ID NO: 7) |
| Heavy Chain CDR3 | NHDNPYNYAMDY | (SEQ ID NO: 8) |
| Light Chain CDR1 | RADKSVSTSTYNYLH | (SEQ ID NO: 12) |
| Light Chain CDR2 | LASNLAS | (SEQ ID NO: 10) |
| Light Chain CDR3 | QHSRDLPPT | (SEQ ID NO: 11) |

Variable regions of the humanized heavy and light chain sequences are shown below:

1. Heavy chain variable regions
Ab-9
(SEQ ID NO: 13)
QVTLKESGPVLVKPTETLTLTCTVSGFSLPNYGVHWVRQPPGKALEWLAV
IWSGGSTNYAAAFVSRLRISKDTSKSQVVFTMNNMDPVDTATYYCARNHD
NPYNYAMDYWGQGTTVTVSS
Ab-10
(SEQ ID NO: 14)
QVQLVESGGGVVQPGRSLRLSCAASGFSLSNYGVHWVRQAPGKGLEWLAV
IWSGGSTNYAAAFVSRLTISKDNSKNTVYLQMNSLRAEDTAVYYCARNHD
NPYNYAMDYWGQGTTVTVSS
Ab-11
(SEQ ID NO: 15)
QVQLVESGGGVVQPGRSLRLSCAASGFTLPNYGVHWVRQAPGKGLEWLAV
IWSGGSTNYAAAFVSRLTISKDNSKNTVYLQMNSLRAEDTAVYYCARNHD
NPYNYAMDYWGQGTTVTVSS
2. Light chain variable regions
Ab-9
(SEQ ID NO: 16)
DIVLTQSPASLAVSPGQRATITCRANKSVSTSTYNYLHWYQQKPGQPPKL
LIYLASNLASGVPARFSGSGSGTDFTLTINPVEANDTANYYCQHSRDLPP
TFGQGTKLEIKR
Ab-10
(SEQ ID NO: 17)
DIVLTQSPDSLAVSLGERATINCRADKSVSTSTYNYLHWYQQKPGQPPKL
LIYLASNLASGVPDRFSGSGSGTDFTLTISSLQAEDVAVYYCQHSRDLPP
TFGQGTKLEIKR
Ab-11
(SEQ ID NO: 18)
DIVLTQSPDSLAVSLGERATINCRANKSVSTSTYNYLHWYQQKPGQPPKL
LIYLASNLASGVPDRFSGSGSGTDFTLTISSLQAEDVAVYYCQHSRDLPP
TFGQGTKLEIKR

The humanized heavy and light chain sequences were recombined with IgG Fc regions to obtain the humanized anti c-Met monoclonal antibody of the invention. The Fc sequence used was selected optionally from the following sequences:

Heavy chain constant region:
(SEQ ID NO: 19)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(SEQ ID NO: 20)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK
(SEQ ID NO: 21)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Light chain constant region:
(SEQ ID NO: 22)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

The above antibodies were cloned, expressed and purified by gene cloning and recombinant expression, respectively. The humanized antibodies Ab-9, Ab-10 and Ab-11 with best activity were finally selected by ELISA (Example 2) and in vitro binding activity assay (Example 6). The sequences are shown below:

Ab-9 humanized antibody:
Heavy chain:
(SEQ ID NO: 23)
QVTLKESGPVLVKPTETLTLTCTVSGFSLPNYGVHWVRQPPGKALEWLAV
IWSGGSTNYAAAFVSRLRISKDTSKSQVVFTMNNMDPVDTATYYCARNHD
NPYNYAMDYWGQGTTVTVSSASTKGPSVFPLAPCSRSTSESTAALGCLVK
DYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTL
MISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
Light chain:
(SEQ ID NO: 26)
DIVLTQSPASLAVSPGQRATITCRANKSVSTSTYNYLHWYQQKPGQPPKL
LIYLASNLASGVPARFSGSGSGTDFTLTINPVEANDTANYYCQHSRDLPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
Ab-10 humanized antibody:
Heavy chain:
(SEQ ID NO: 24)
QVQLVESGGGVVQPGRSLRLSCAASGFSLSNYGVHWVRQAPGKGLEWLAV
IWSGGSTNYAAAFVSRLTISKDNSKNTVYLQMNSLRAEDTAVYYCARNHD
NPYNYAMDYWGQGTTVTVSSASTKGPSVFPLAPCSRSTSESTAALGCLVK
DYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTL
MISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
Light chain:
(SEQ ID NO: 27)
DIVLTQSPDSLAVSLGERATINCRADKSVSTSTYNYLHWYQQKPGQPPKL
LIYLASNLASGVPDRFSGSGSGTDFTLTISSLQAEDVAVYYCQHSRDLPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
Ab-11 humanized antibody:
Heavy chain:
(SEQ ID NO: 25)
QVQLVESGGGVVQPGRSLRLSCAASGFTLPNYGVHWVRQAPGKGLEWLAV
IWSGGSTNYAAAFVSRLTISKDNSKNTVYLQMNSLRAEDTAVYYCARNHD
NPYNYAMDYWGQGTTVTVSSASTKGPSVFPLAPCSRSTSESTAALGCLVK
DYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTL
MISRTPEVTCVVVDVSHEDPEVQFNWYVDGVEVHNAKTKPREEQFNSTFR
VVSVLTVVHQDWLNGKEYKCKVSNKGLPAPIEKTISKTKGQPREPQVYTL
PPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
Light chain:
(SEQ ID NO: 28)
DIVLTQSPDSLAVSLGERATINCRANKSVSTSTYNYLHWYQQKPGQPPKL
LIYLASNLASGVPDRFSGSGSGTDFTLTISSLQAEDVAVYYCQHSRDLPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

### Example 6. Detection of In Vitro Binding Activity of Anti c-Met Humanized Antibody

The humanized antibodies of the invention were analyzed for their in vitro activity by ELISA (Example 2), and also analyzed for their binding with the cell line MKN45, which highly expresses c-Met, and for their affinity to c-Met antigen (BIACore assay).

A FACS method was used to detect the binding activity of c-Met humanized antibodies with the cell line MKN45, which highly expresses c-Met.

MKN45 cells (JCRB, Cat No.: JCRB0254) were resuspended in RPMI1640 medium (GIBCO, Cat No.: 11835-030) which contains 10% (v/v) fetal calf serum (FBS GIBCO, Cat No.: 10099-141) and Penicillin/Streptomycin (GIBCO, Cat No.: 15070-063) to reach 10,000,000 cells/mL. 2 mL of resuspended MKN45 cells was added to 96-well microtiter plate (Corning, Cat No.: 3799) at 150,000 cells/well, and 8 concentrations of c-Met antibody (5-fold gradient diluted, starting from 20 μg/mL) were added to the corresponding wells, and the final volume was 100 μL. The plate was incubated for 1 hour at 4° C. FACS buffer (PBS comprising 2.5% (v/v) FBS (Hyclone, Cat: SH30256.01B)) was added. The plate was centrifuged under 4° C. at 1300 rmp for 4 minutes, and the supernatant was discarded. This procedure was repeated three times. 100 μL of secondary antibodies (Fluorescence labeled goat-anti-mouse secondary antibodies with 1:200 dilution, Biolegend, Cat No. 405307; Fluorescence labeled anti-human secondary antibody with 1:30 dilution, Biolegend, Cat No. 409304) were added to each well, and the plate was incubated for 1 hour at 4° C. FACS buffer was added, the plate was centrifuged under 4° C. at 1300 rpm for 4 minutes, the supernatants were discarded; and this procedure was repeated three times. 200 L FACS buffer was added to resuspend the cells, and the prepared samples were detected by flow cytometry (BD FACS Array).

The affinity of c-Met antibody to c-Met antigen Sema-His was detected by surface plasmon resonance (SPR) in the invention.

Anti-mouse IgG (GE Life Sciences catalog # BR-1008-38) or anti-human IgG (GE Life Sciences catalog # BR-1008-39) antibodies were respectively diluted to 30 μg/mL and 50 μg/mL by sodium acetate solution pH 5.0 (GE Healthcare, Cat # BR-1003-51). An amino coupling kit (GE Life Sciences, Cat # BR100050) was immobilized onto the test channels and control channels on a CM5 chip (GE Life Sciences catalog # BR-1000-12), and the coupling level was set at 15000 RU. The c-Met antibody was diluted with Running buffer PBS (Hyclone, Cat # SH30256.01B)+0.05% P20 (GE Life Sciences, Cat # BR-1000-54) to 1.5 μg/mL. Antigen Sema-His was diluted to 200 nM with running buffer, and then diluted at a 1:2 dilution with the same buffer until 0.78 nM was reached. The diluted antibody passed through the test channel for 1 minute at a speed of 30 μL/min, and the antigen passed through the test channels and control channels for 3 minutes at the same speed. 10 minutes after dissociation, the flow speed was adjusted to 10 μL/min, and regeneration buffer was passed through test channels and control channels for 3 minutes. Data was fitted by BiaEvaluation 4.1 after double deduction, and the fitting model was a 1:1 Langmuir model.

Experimental Results:

TABLE 4
Binding activity of humanized anti c-Met antibodies

| | Humanized antibody | | |
|---|---|---|---|
| | Ab-9 | Ab-10 | Ab-11 |
| ELISA assay (EC₅₀, nM) | 0.13 | 0.39 | 0.2 |

TABLE 5
Binding activity and affinity of humanized anti c-Met antibodies to
MKN45 cells and to antigen

| | MKN45/FCAS Binding | affinity to antigen |
|---|---|---|
| Humanized antibody | activity (nM) | Biacore(nM) |
| Ab-9 | 1.6 | 4 |
| Ab-10 | 1.23 | 8 |

Conclusion: The above experimental results show that the binding activity of humanized antibodies with antigen was within 0.13-8 nM, and the results may vary depending on the detection methods used. The results show that humanized anti c-Met antibodies maintain the binding activity of the parent antibodies prior to humanization.

### Example 7. Endocytosis of Anti c-Met Antibody

Antibodies of the invention bind to human c-Met, and have very good in vitro activity and good activity in inhibiting tumor activity in vivo. In addition, the antibodies do not have agonist activity, or have very weak agonist activity. In order to detect whether the antibodies would be internalized into the cell along with human c-Met once bound to human c-Met, human gastric cancer cell line MKN45 (JCRB, Cat No.: JCRB0254) expressing c-Met was used for evaluation.

MKN45 cells were resuspended to 10,000,000 cells/mL in RPMI 1640 medium (GIBCO, Cat No.: 11835-030), which contains 10% (v/v) FBS (GIBCO, Cat No.: 10099-141) and penicillin/streptomycin (GIBCO, Cat No.: 15070-063). 2 mL resuspended MKN45 cells were added to a 96-well microtiter plate with 250,000 cells/well, and 10 μg/mL of c-Met antibody was added to the corresponding wells and the final volume was 100 μL. The plate was incubated at 4° C. for 1 hour. FACS buffer (phosphate buffer solution including 2.5% fetal bovine serum; Hyclone, Cat: SH30256.01B) was added and the plate was centrifuged at 4° C., 1300 rpm for 4 minutes. The supernatant was discarded and this procedure was repeated three times. 100 μL secondary antibody solution (Fluorescence labeled goat anti mouse secondary antibodies at 1:200 dilution, Biolegend, Cat #405307; Fluorescence labeled anti-human secondary antibody at 1:30 dilution, Biolegend, Cat #409304) was added into each well. The plate was incubated at 4° C. for 1 hour. FACS buffer was added and the plate was centrifuged at 4° C., 1300 rpm for 4 minutes. The supernatant was discarded and this procedure was repeated three times. Complete cell culture medium (RPMI 1640 medium with 10% FBS) was added and was incubated at 37° C. in 5% CO₂ for 0, 0.5, 1, 2, 4 hours. 5 μL 7-AAD (Biolegend, Cat:420403) was added to 100 μL FACS buffer which was added to each well, and the plate was incubated at 4° C. for 30 minutes. FACS buffer was added and the plate was centrifuged at 4° C., 1300 rpm for 4 minutes. The supernatant was discarded and this procedure was repeated three times. 200 μL Stripping buffer (0.05 M glycine, pH 3.0; 0.1 M NaCl, mixed at 1:1 (v/v)) was added to each well. The cells were resuspended and were incubated for 7 minutes at room temperature. The cells were centrifuged at room temperature at 1300 rpm for 4 minutes, and the supernatant was discarded. 200 μL neutralizing wash buffer (0.15M trihydroxymethyl aminomethane, pH 7.4) was added to each well, the cells were resuspended and centrifuged at room temperature at 1300 rpm for 4 minutes, and the supernatant was discarded. 200 μL FACS buffer was added and the cells were resuspended. The prepared samples were detected by flow cytometry (BD FACS Calibur). The results are shown in the table below.

Endocytosis of c-Met antibody %=(intensity of fluorescence at each time point−mean intensity of fluorescence at time 0)/mean intensity of fluorescence at time 0.

Experimental Results:

TABLE 6
Evaluation of endocytosis of humanized anti c-Met antibodies of the
present invention (endocytosis %)

| Humanized | | | | | |
|---|---|---|---|---|---|
| antibody | 0 h | 0.5 h | 1 h | 2 h | 4 h |
| hIgG (control)* | 0 | −0.9 | −4.4 | −4.9 | 3.6 |
| Ab-9 | 0 | 26 | 32 | 32 | 31 |
| Ab-10 | 0 | 24 | 38 | 53 | 59 |

*−4.9% and −3.6% in control group were due to experimental error (background value), and was classified as no endocytosis.

Experimental Conclusion:

The experimental results in the table above show that antibodies of the invention have good endocytosis while they do not have agonist activity. Once bound with target cells, both antibodies and receptors were rapidly internalized into target cells, and the maximum value was reached within 2-4 hours.

### Example 8. Analysis of the Biophysical Stability of Anti c-Met Antibodies

To evaluate the biophysical stability of the anti c-Met antibodies of the invention, such as the presence of glycosylation and deamination sites and stability, LC-MS analysis was used.

The molecular weight of the heavy and light chains was directly detected by LC-MS to analyze glycosylation. Deamination was analyzed by LC-MS at 4° C. for long time (at least 3 months), or at 40° C. for 21 days under an accelerated condition. Samples treated with different conditions were diluted to 2 mg/mL with pH 7.2 Tris-HCl; final concentrations of 10 mM of TCEP and 6M of urea (AMRESCO, Cat #0378)₃ were added, then the samples were incubated for 20 minutes at 37° C. IAA (Sigma-Aldrich, Cat # I1149) with a final concentration of 20 mM was added and was incubated for 15 minutes in darkness to protect the sulfhydryl group. The pH of the sample was adjusted by dilution with Tris-HCl, pH 7.2, and protease (Sigma-Aldrich, Cat # T6567) was added at a ratio by weight of 10:1 (protein: enzyme). The samples were incubated at 37° C. for 25 minutes, and then formic acid with a final concentration of 0.1% (Fluca, Cat #94318) was added to terminate the reactions. Samples were centrifuged and analyzed by LC-MS.

BiopharmaLynx was used to analyze the presence of deamination. Extracted Ion Chromatogram (EIC) was obtained from MS data by searching native peptide comprising deamination site and modified product, and then extracting parent ion. Peak area was obtained by integration, and the percentages of deamination and oxidation product were calculated.

Experimental Results:

TABLE 7
Evaluation of physical stability of the humanized anti c-Met antibody
of the invention

| | | | Analysis | |
|---|---|---|---|---|
| The antibody | | | of deamination# | |
| of the | Molecular weight of light chain* | | 4° C., | 40° C., |
| invention | Detected value | Estimated value | 3.5 months | 21 days |
| Ab-9 | 25940 | 23907 | 0.66 | — |
| Ab-10 | 23828 | 23832 | — | 0.3 |

*Heavy chains all involve glycosylation, and molecular weight was consistent with the expected value.
#Percentage of deaminated molecules (%). 0.66-1.0% is within the background of detection.
—: not tested.

Experimental conclusion: The above results show that the antibodies of the invention are stable and have good physical properties.

### Example 9. Anti-c-Met Antibody Ab-10 Conjugated to Toxin MC-MMAF (No. 1)

The anti-c-Met antibodies of the present invention have inhibitory activity against receptor binding, without having agonist activity, show endocytosis activity into targeted cells, as well as physical stability. These properties make the antibodies of the invention particularly suitable for the preparation of ADC drugs when conjugated to toxins for the treatment of c-Met expressing cancers. The coupling process is shown below:

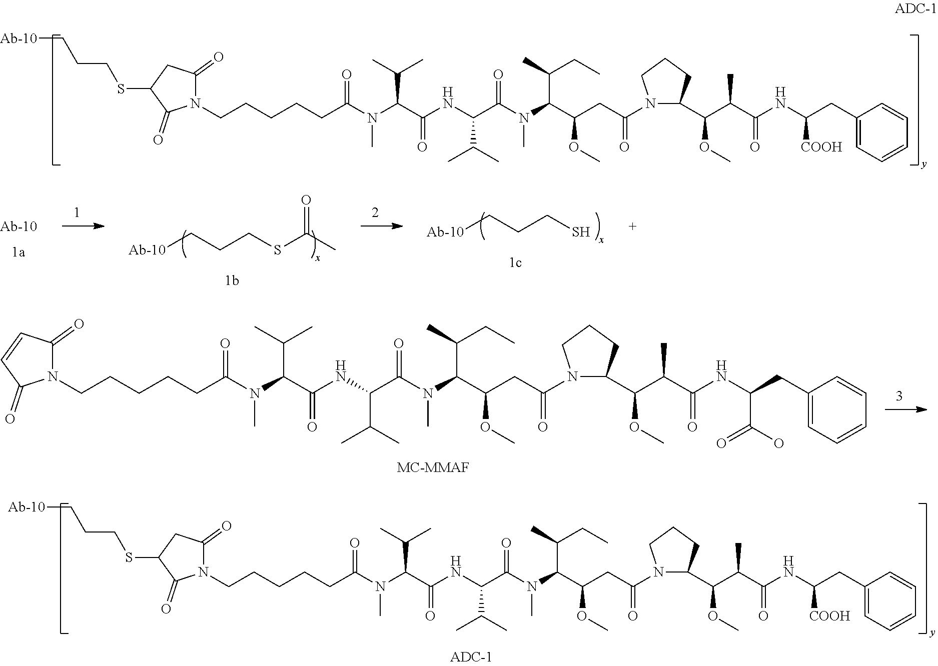

Step 1. Thioacetic acid S-(3-carbonyl propyl) ester (0.7 mg, 5.3 μmol) was dissolved in acetonitrile solution (0.9 mL), for use. The thioacetic acid S-(3-carbonyl propyl) ester in acetonitrile prepared above was added into acetic acid/sodium acetate buffer pH=4.3 (10.35 mg/mL, 9.0 mL, 0.97 mmol) containing Ab-10 monoclonal antibody, and sodium borohydride aqueous solution (14.1 mg, 224 μmol, 1.0 mL) was added dropwise with shaking for 2 hours at 25° C. At the end of the reaction, desalination and purification were done on a Sephadex G25 gel column (Elution phase: 0.05M of PBS solution pH 6.5), and product 1b solution was collected and was concentrated to 10 mg/mL directly for the next reaction.

Step 2. hydroxylamine hydrochloride solution (2.0M, 0.35 mL) was added into 11.0 mL of 1b solution with shaking for 30 minutes at 25° C., and then desalination and purification were done on a Sephadex G25 gel column (Elution phase: 0.05M of PBS solution pH 6.5), and the captioned product Ab-10 monoclonal antibody-propyl mercaptan 1c solution was collected (6.17 mg/mL, 14.7 mL).

Step 3. The compound MC-MMAF (1.1 mg, 1.2 μmol; prepared by method published in PCT patent WO2005081711) was dissolved in acetonitrile (0.3 mL) and was added in Ab-10 monoclonal antibody-propyl mercaptan 1c solution (6.17 mg/mL, 3.0 mL) with shaking for 4 hours at 25° C., and then desalination and purification were done on a Sephadex G25 gel column (Elution phase: 0.05M of PBS solution pH 6.5). The captioned product ADC-1 (3.7 mg/mL, 4.7 mL) in PBS buffer was obtained by filtration through a 0.2 μm filter under aseptic conditions, and then frozen stored at 4° C.

Q-TOF LC/MS: characteristic peak: 148119.2 (M_{Ab}+0D), 149278.1 (M_{Ab}+1D), 150308.1 (M_{Ab}+2D), 151314.1 (M_{Ab}+3D). The amount of the conjugated toxin per antibody (DAR) was calculated by analysis and the mean value was y=1.7.

### Example 10. Anti-c-Met Antibody Ab-10 Conjugated with Toxin MC-VC-PAB-MMAE (No. 2)

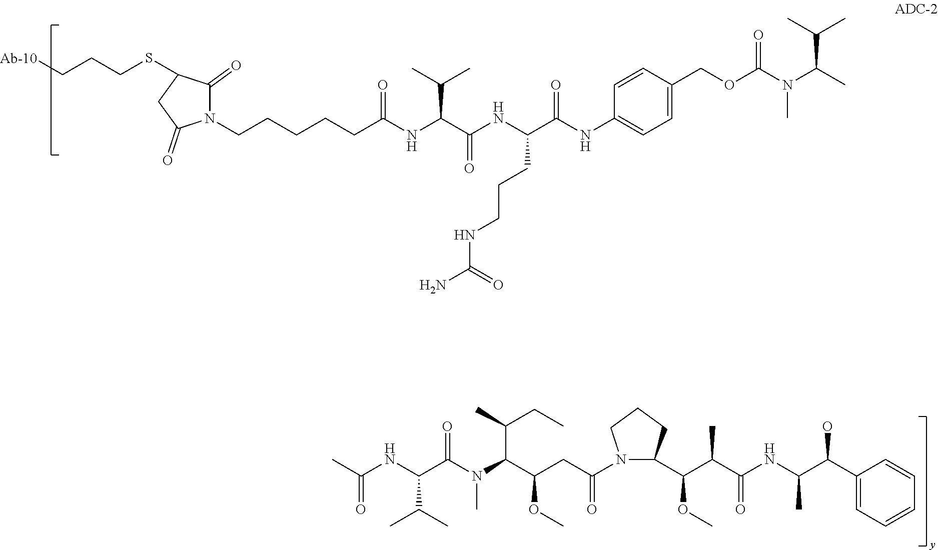

The compound MC-VC-PAB-MMAE (1.6 mg, 1.2 μmol; prepared by the method disclosed in PCT patent application WO2004010957) was dissolved in acetonitrile (0.3 mL) and was added into Ab-10 monoclonal antibody-propyl mercaptan 1c solution (6.17 mg/mL, 3.0 mL) with shaking for 4 hours at 25° C., and then desalination and purification were done on a Sephadex G25 gel column (Elution phase: 0.05M of PBS solution, pH 6.5). The captioned product ADC-2 in PBS buffer (3.6 mg/mL, 4.8 mL) was obtained by filtration through a 0.2 μm filter under aseptic conditions, and then frozen stored at 4° C.

Q-TOF LC/MS: characteristic peak: 148118.4 (M_{Ab}+0D), 149509.2 (M_{Ab}+1D), 150903.1 (M_{Ab}+2D), 152290.4 (M_{Ab}+3D), 153680.7 (M_{Ab}+4D). The amount of the conjugated toxin per antibody (DAR) was calculated by analysis and the mean value is y=1.8.

### Example 11. Anti-c-Met Antibody Ab-10 Conjugated with Toxin MC-VC-PAB-MMAF (No. 3)

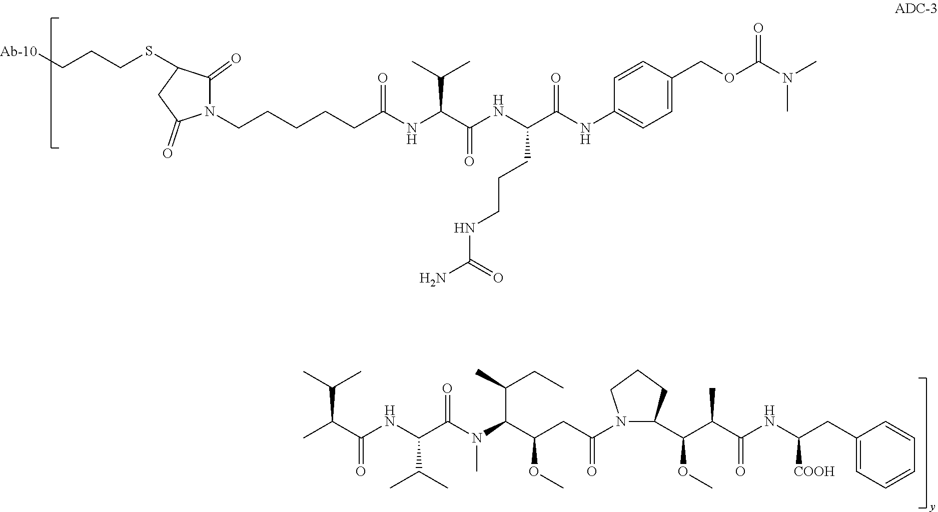

The compound MC-VC-PAB-MMAF (1.6 mg, 1.2 μmol; prepared as method disclosed in PCT patent application WO2005081711) was dissolved in acetonitrile (0.3 mL) and was added to Ab-10 monoclonal antibody-propyl mercaptan 1c solution (6.17 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-3 (3.5 mg/mL, 4.9 mL) in PBS buffer was obtained by filtration through a 0.2 m filter under aseptic conditions, and then frozen stored at 4° C.

Q-TOF LC/MS: characteristic peak: 148119.1 (M_{Ab}+0D), 149525.3 (M_{Ab}+1D), 150930.7 (M_{Ab}+2D), 152335.2 (M_{Ab}+3D), 153739.8 (M_{Ab}+4D). The amount of the conjugated toxin per antibody (DAR) was calculated by analysis and the mean value was y=1.6.

### Example 12. Anti-c-Met Antibody Ab-10 Conjugated Toxin with MC-MMAE (No. 4)

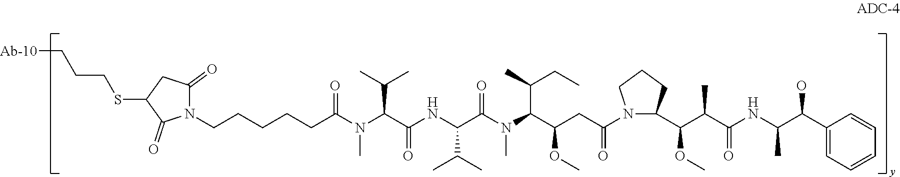

The compound MC-MMAE (1.2 mg, 1.2 μmol; prepared as method disclosed in patent application US7/750/116B1) was dissolved in acetonitrile (0.3 mL) and was added to Ab-10 monoclonal antibody-propyl mercaptan 1c solution (6.17 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-4 in PBS buffer (3.4 mg/mL, 5.0 mL) was obtained by filtration through a 0.2 μm filter under aseptic condition, and then frozen stored at 4° C.

Q-TOF LC/MS: characteristic peak: 148118.6 (M_{Ab}+0D), 149104.3 (M_{Ab}+1D), 150090.1 (M_{Ab}+2D), 151075.8 (M_{Ab}+3D). The amount of the conjugated toxin per antibody (DAR) was calculated by analysis and the mean value was y=1.6.

### Example 13. Anti-c-Met Antibody Ab-9 Conjugated with Toxin MC-MMAE (No. 5)

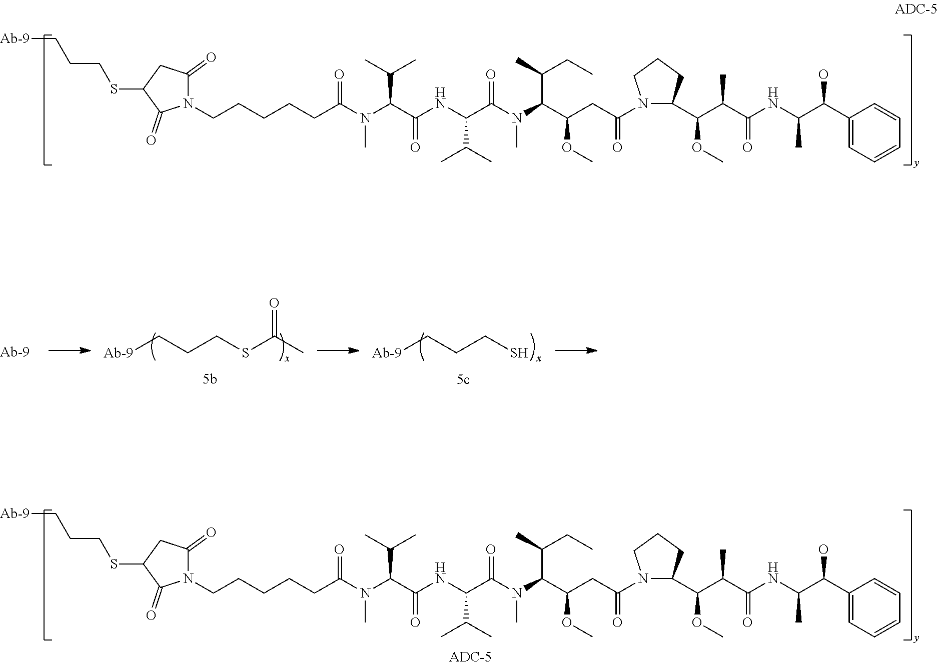

Step 1. Thioacetic acid S-(3-carbonyl propyl) ester (0.7 mg, 5.3 μmol) was dissolved in 0.9 mL acetonitrile solution, for use. The thioacetic acid S-(3-carbonyl propyl) ester in acetonitrile prepared above was added into acetic acid/sodium acetate buffer containing Ab-9 monoclonal antibody (10.85 mg/mL, 9.0 mL, 0.976 mmol), and sodium borohydride aqueous solution (14.1 mg, 224 μmol, 1.0 mL) was added dropwise with shaking for 2 hours at 25° C. At the end of the reaction, desalination and purification were done on a Sephadex G25 gel column (Elution phase: 0.05M of PBS solution which pH is 6.5), and the captioned product 5b solution was collected and concentrated to 10 mg/mL directly for the next reaction.

Step 2. hydroxylamine hydrochloride solution (2.0M, 0.35 mL) was added into 5b solution (11.0 mL) with shaking for 30 minutes at 25° C., and then desalination and purification were done on a Sephadex G25 gel column (Elution phase: 0.05M of PBS solution which pH is 6.5), and the captioned product Ab-9 monoclonal antibody-propyl mercaptan 5c solution was collected (6.2 mg/mL, 15.0 mL).

Step 3. the compound MC-MMAE (1.1 mg, 1.2 mol) was dissolved in acetonitrile (0.3 mL) and was added to Ab-9 monoclonal antibody-propyl mercaptan 5c solution (6.2 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-5 in PBS buffer (3.8 mg/mL, 4.6 mL) was obtained by filtration through a 0.2 μm filter under aseptic condition, and then frozen stored at 4° C.

Q-TOF LC/MS: characteristic peak: 150530.9 (M_{Ab}+OD), 151915.7 (M_{Ab}+1D), 153333.6 (M_{Ab}+2D), 154763.4 (M_{Ab}+3D), 156271.9 (M_{Ab}+4D). The amount of conjugated toxin per antibody (DAR) was calculated by analysis and the mean value was y=1.5.

### Example 14. Anti-c-Met Antibody Ab-9 Conjugated with Toxin MC-MMAF (No. 6)

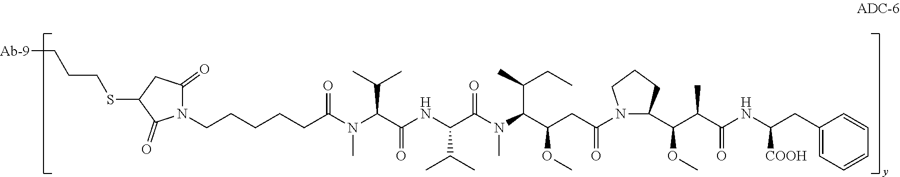

The compound MC-MMAF (1.1 mg, 1.2 mol) was dissolved in acetonitrile (0.3 mL) and was added to Ab-9 monoclonal antibody-propyl mercaptan 5c solution (6.17 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-6 in PBS buffer (3.8 mg/mL, 4.6 mL) was obtained by filtration through a 0.2 m filter under aseptic condition, and then frozen stored at 4° C.

Q-TOF LC/MS: characteristic peak: 150537.8 (M_{Ab}+OD), 152087.9 (M_{Ab}+1D), 153486.5 (M_{Ab}+2D), 154911.7 (M_{Ab}+3D), 156499.9 (M_{Ab}+4D). The amount of conjugated toxin per antibody (DAR) was calculated by analysis and the mean value was y=1.7.

### Example 15. Anti-c-Met Antibody Ab-9 Conjugated with Toxin MC-VC-PAB-MMAF (No. 7)

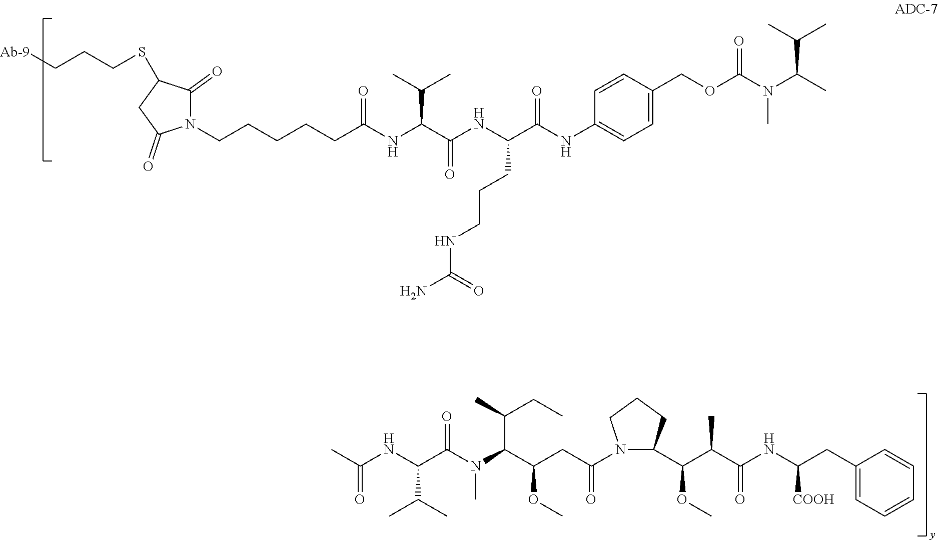

The compound MC-VC-PAB-MMAF (1.6 mg, 1.2 mol) was dissolved in acetonitrile (0.3 mL) and was added to Ab-9 monoclonal antibody-propyl mercaptan 5c solution (6.2 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-7 in PBS buffer (3.8 mg/mL, 4.6 mL) was obtained by filtration through a 0.2 μm filter under aseptic condition, and then stored at 4° C.

Q-TOF LC/MS: characteristic peak: 150537.8 (M_{Ab}+0D), 152087.9 (M_{Ab}+1D), 153486.5 (M_{Ab}+2D), 154911.7 (M_{Ab}+3D), 156499.9 (M_{Ab}+4D). The amount of conjugated toxin per antibody (DAR) was gained by analysis and the mean value was y=1.8.

### Example 16. Anti-c-Met Antibody Ab-9 Conjugated with Toxin MC-VC-PAB-MMAE (No. 8)

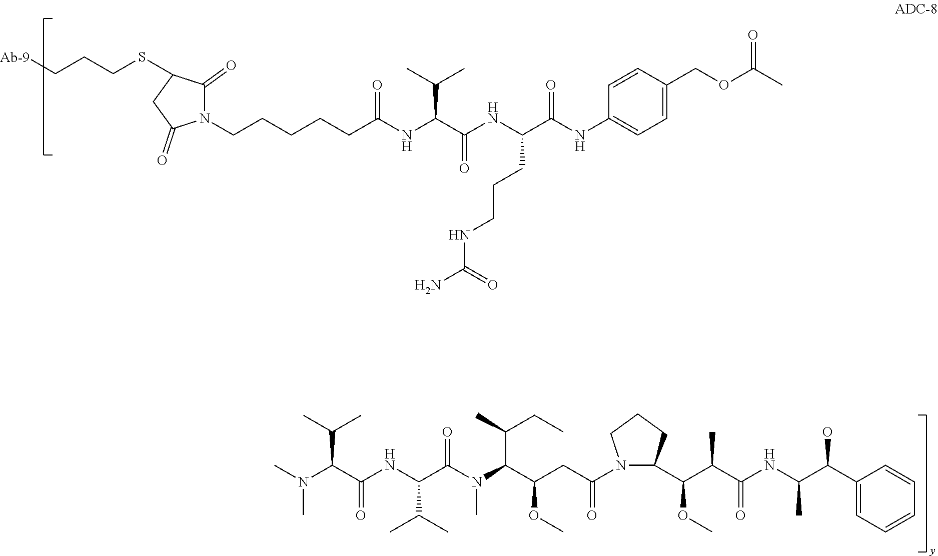

The compound MC-VC-PAB-MMAE (1.6 mg, 1.2 μmol) was dissolved in acetonitrile (0.3 mL) and was added to Ab-9 monoclonal antibody-propyl mercaptan 5c solution (6.2 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-8 in PBS buffer (3.8 mg/mL, 4.6 mL) was obtained by filtration through a 0.2 μm filter under aseptic condition, and then frozen stored at 4° C.

Q-TOF LC/MS: characteristic peak: 150508.6 (M_{Ab}+OD), 151903.6 (M_{Ab}+1D), 153314.5 (M_{Ab}+2D), 154747.8 (M_{Ab}+3D), 156039.5 (M_{Ab}+4D). The amount of conjugated toxin per antibody (DAR) was gained by analysis and the mean value was y=1.6.

### Example 17. Anti-c-Met Antibody Ab-9 Conjugated with Toxin SN-38 (No. 9)

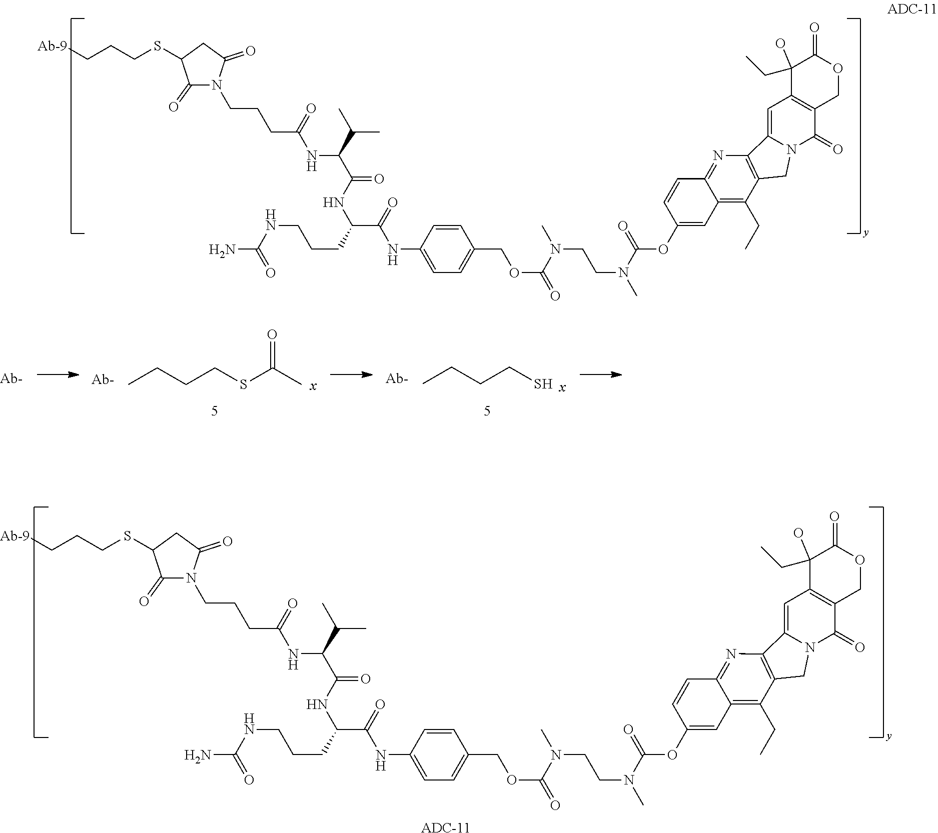

The compound MC-VC-PAB-SN-38 (1.3 mg, 1.2 μmol) was dissolved in acetonitrile (0.3 mL) and was added to Ab-9 monoclonal antibody-propyl mercaptan 5c solution (6.2 mg/mL, 3.0 mL) with shaking for 4 hours at 25° C., and then desalination and purification were done on a Sephadex G25 gel column (Elution phase: 0.05M of PBS solution which pH is 6.5). The captioned product ADC-11 in PBS buffer (3.7 mg/mL, 4.5 mL) was obtained by filtration through a 0.2 m filter under aseptic condition, and then stored at 4° C.

Q-TOF LC/MS: characteristic peak: 150537.1 (M_{Ab}+0D), 151786.6 (M_{Ab}+1D), 152948.6 (M_{Ab}+2D), 154161.7 (M_{Ab}+3D), 155365.9 (M_{Ab}+4D), 156477.8 (M_{Ab}+5D).

The mean value was y=2.6.

### Example 18. Anti-c-Met Antibody Ab-10 Conjugated with Toxin (No. 10)

### 1. Preparation of Toxin

### (S)-2-((2R,3R)-3-((1S,3S,5S)-2-((3R,4S,5S)-4-((S)—N,3-dimethyl-2-((S)-3-methyl-2-(methyl amino)butyramide)butyramide)-3-methoxy-5-methylheptanoyl)-2-azabicyclo[3.1.0]hexane-3-yl)-3-methoxy-2-methylpropanamide)-3-(2-fluorophenyl) propionic acid

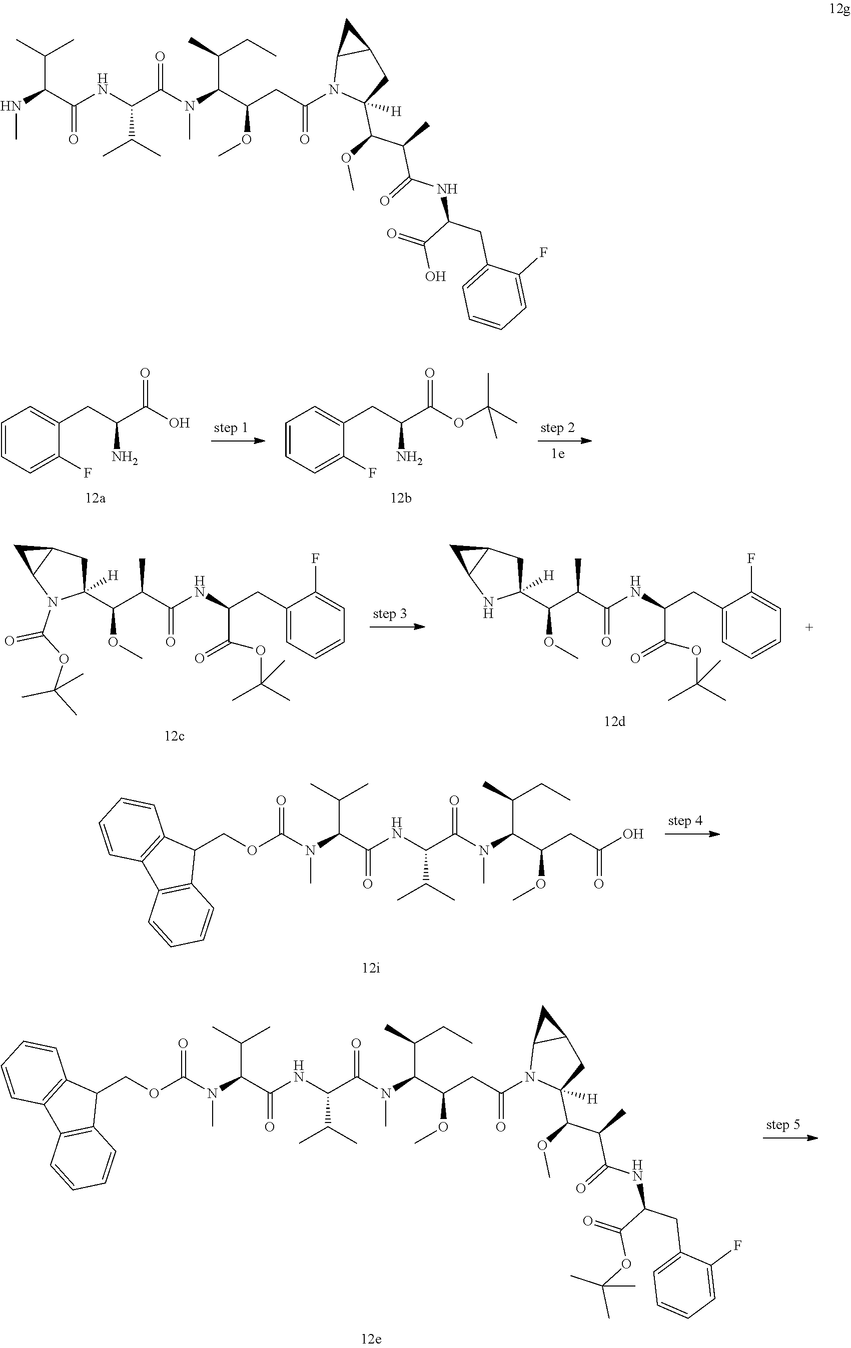

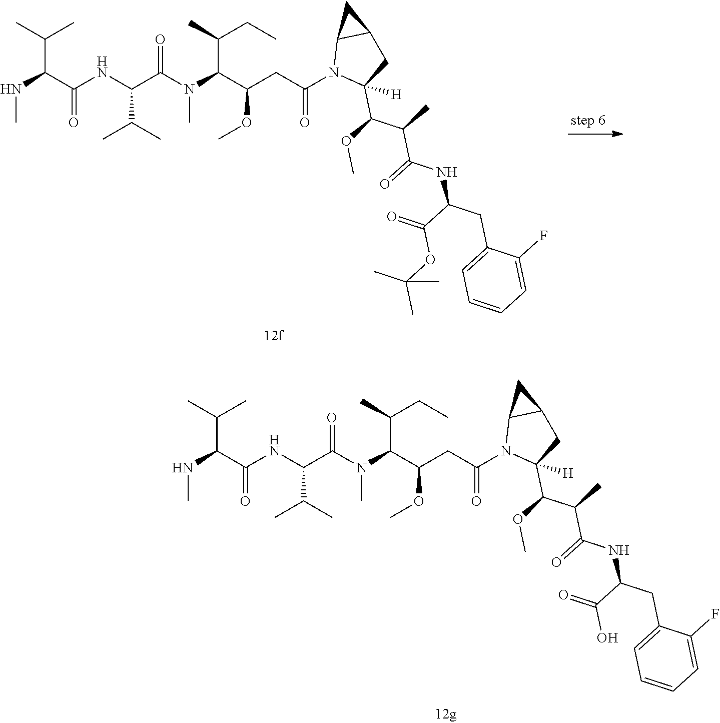 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)OC(C)(C)C)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C]

### Step 1

### Preparation of (S)-tert-butyl-2-amino-3-(2-fluorophenyl) propanoic acid

Starting material (S)-2-amino-3-(2-fluorophenyl)propanoic acid 12a (400 mg, 2.18 mmol, prepared according to the known method in “Advanced Synthesis & Catalysis, 2012, 354(17), 3327-3332”) was dissolved in 10 mL of tert-butyl acetate. Perchloric acid (300 mg (70%), 3.3 mmol) was added and stirred at room temperature for 16 hours. Water (6 mL) was added after the reaction, and the solution was separated. The organic phase was washed with saturated sodium bicarbonate solution (5 mL). The aqueous phase was adjusted to pH=8 with saturated sodium bicarbonate solution, and was then extracted with dichloromethane (5 mL×3), and the organic phase was combined. The reaction mixture was then washed successively with water (3 mL) and saturated sodium chloride solution (5 mL), dried with anhydrous sodium sulfate, filtered; and the filtrate was concentrated under reduced pressure. The crude product compound 12b was obtained (390 mg, yellow, oily) and was subjected to the next reaction directly without purification.

### Step 2

### Preparation of (1S,3S,5S)-tert-butyl 3-((1R,2R)-3(((S)-1-(t-butoxy)-3-(2-fluorophenyl)-1-carbonylpropyl-2-yl)amino)-1-methoxy-2-methyl-3-carbonyl propyl)-2-azabicyclo[3.1.0]hexane-2-carboxylic acid

The starting material (2R,3R)-3-((1S,3S,5S)-2-(tert-butoxycarbonyl)-2-azabicyclo[3.1.0]hexane-3-yl)-3-methoxy-2-methyl propionate 12e (100 mg, 0.334 mmol) was dissolved in the mixture of dichloromethane (6 mL) and dimethylformamide (V/V=5:1), and then crude product (S)-tert-butyl 2-amino-3-(2-fluorophenyl) propionate 12b (80 mg, 0.334 mmol) was added. N,N-diisopropylethylamine (0.29 mL, 1.67 mmol) and 2-(7-azabenzotriazol)-N,N,N′,N′-tetramethyluronium hexafluorophosphate (152.3 mg, 0.40 mmol) were added to the mixture. The mixture was stirred for 1 hour under argon atmosphere at room temperature. After the reaction, water (10 mL) was added and stirred, and layers were separated. The layer of dichloromethane was washed by saturated sodium chloride solution (10 mL), and dried with anhydrous sodium sulfate, and filtered, and the filtrate was concentrated under reduced pressure. The residues were purified by silica gel column chromatography using eluent system B to obtain the captioned product compound 12c (173 mg, clear liquid, the yield was 99.5%).

MS m/z (ESI): 521.2 [M+1]

### Step 3

### Preparation of (S)-tert-butyl-2-((2R,3R)-3-((1S,3S,5S),-2-azabicyclo[3.1.0]hexane-3-yl)-3-methoxy-2-methylpropionamide)-3-(2-fluorophenyl) propionic acid

The starting material (1S,3S,5S)-tert-butyl-3-((1R,2R)-3-(((S)-1-(t-butoxy)-3(2-fluorophenyl)-1-carbonylpropyl-2-yl)amino)-1-methoxy-2-methyl-3-carbonyl propyl)-2-azabicyclo[3.1.0]hexane-2-carboxylic acid 12c (173 mg, 0.33 mmol) was dissolved in dioxane (2 mL), and hydrogen chloride dioxane solution (5.6M, 0.21 mL, 1.16 mmol) was added. The mixture was stirred for 1 hour under argon atmosphere at room temperature, and was placed in a 0° C. refrigerator for 12 hours. After the reaction, the reaction mixture was concentrated under reduced pressure, and dichloromethane (5 mL) was added to dilute the reaction mixture. Saturated sodium bicarbonate solution (10 mL) was added and the mixture was stirred for 10 minutes. The product was layered and the aqueous phase was extracted by dichloromethane (5 mL×3). Dichloromethane layers were combined and were washed by saturated sodium chloride solution (10 mL), dried with anhydrous sodium sulfate, and filtered. The filtrate was concentrated under reduced pressure. The crude product of the captioned compound 12d (77 mg, yellow liquid) was obtained and directly subjected to the next reaction without purification.

MS m/z (ESI):421.2 [M+1]

### Step 4

### Preparation of (S)-tert-butyl-2-((2R,3R)-3-((1S,3S,5S)-2-(5S,8S,11S,12R)-11-((S)-sec-butly)-1-(9H-fluorene-9-yl)-5,8-diisopropyl-12-methoxy-4,10-dimethyl-3,6,9-tricarbonyl-2-oxygen-4,7,10-triazatetradecyl-14-acyl)-2-azabicyclo[3.1.0]hexane-3-yl)-3-methoxy-2-methylpropionamide)-3-(2-fluorophenyl) propionic acid

Crude product (S)-tert-butyl-2-((2R,3R)-3-((1S,2S,5S)-2-azabicyclo[3.1.0]hexane-3-yl)-3-methoxy-2-methylpropionamide)-3-(2-fluorophenyl) propionic acid 12d (77 mg, 0.183 mmol) and (5S,8S,11S,12R)-11-((S)-sec-butyl)-1-(9H-fluorene-9-yl)-5,8-diisopropyl-12-methoxy-4,10-dimethyl-3,6,9-tricarbonyl-2-oxo-4,7,10-triazatetradecyl-14-carboxylic acid 12i (116.8 mg, 0.183 mmol, prepared by methods published in patent application “WO 2013072813”) were dissolved in a mixture of dichloromethane (6 mL) and dimethylformamide (V/V=5:1). N,N-diisopropylethylamine (0.16 mL, 0.915 mmol) and 2-(7-azabenzotriazol)-N,N,N′,N′-tetramethyluronium hexafluorophosphate (84 mg, 0.22 mmol) were added to the mixture. The reaction mixture was stirred for 1 hour under argon atmosphere at room temperature. After the reaction, water (10 mL) was added and stirred, and layers were separated. The layer of dichloromethane was washed by saturated sodium chloride solution (10 mL), dried with anhydrous sodium sulfate, and filtered, and the filtrate was concentrated under reduced pressure. The residues were purified by silica gel column chromatography using eluent system B to obtain the captioned product compound 12e (190.5 mg, yellow viscous) with a yield of 100%.

MS m/z (ESI): 1040.6 [M+1]

### Step 5

### Preparation of (S)-tert-butyl-2-((2R,3R)-3-((1S,3S,5S)-2-((3R,4S,5S)-4-((S)—N,3-dimethyl-2-((5)-3-methyl-2-(methylamino)butanamide)butanamide)-3-methoxy-5-methylheptanoyl)-2-azabicyclo[3.1.0]hexane-3-yl)-3-methoxy-2-methylpropanamide)-3-(2-fluorophenyl) propionic acid

The starting material (S)-tert-butyl-2-((2R,3R)-3-((1S,3S,5S)-2-((5S,8S,11S,12R)-11-((S)-sec-butly)-1-(9H-fluorene-9-yl)-5,8-diisopropyl-12-methoxy-4,10-dimethyl-3,6,9-tricarbonyl-2-oxo-4,7,10-triazatetradecyl-14-acyl)-2-azabicyclo[3.1.0]hexane-3-yl)-3-methoxy-2-methyl propionamide)-3-(2-fluorophenyl) propionic acid 12e (190.5 mg, 0.183 mmol) was dissolved in dichloromethane (1.5 mL) and diethylamine (2 mL) was added. The mixture was stirred for 3 hours under argon atmosphere at room temperature. After the reaction, the reaction mixture was concentrated under reduced pressure and the crude captioned product compound 12f (150 mg, yellow viscous) was obtained. Products were directly subjected to the next reaction without purification.

MS m/z (ESI): 818.5 [M+1]

### Step 6

### (S)-2-((2R,3R)-3-((1S,3S,5S)-2-((3R,4S,5S)-4-((S)—N,3-dimethyl-2-((S)-3-methyl-2-(methylamino)butanamide)butanamide)-3-methoxy-5-methylheptanoyl)-2-azabicyclo[3.1.0]hexane-3-yl)-3-methoxy-2-methylpropanamide)-3-(2-fluorophenyl)propionic acid

The crude product compound (S)-tert-butyl-2-((2R,3R)-3-((1 S,3 S,5S)-2-((3R,4S,5S)-4-((S)—N,3-dimethyl-2-((S)-3-methyl-2-(methylamino)butanamide)butanamide)-3-methoxy-5-methylheptanoyl)-2-azabicyclo[3.1.0]hexane-3-yl)-3-methoxy-2-methylpropanamide)-3-(2-fluoro phenyl)propionic acid 12f (150 mg, 0.183 mmol) was dissolved in dioxane (1 mL), and hydrogen chloride in dioxane (5.6M, 3 mL) was added. The mixture was stirred for 12 hours under argon atmosphere at room temperature. After the reaction, the reaction solution was concentrated under reduced pressure with ether solvent. The residues were purified by high performance liquid chromatography to obtain the captioned product compound 12g (28 mg, white powder with yield of 20%).

MS m/z (ESI): 762.7[M+1]

¹H NMR (4.0 MHz, CD₃OD): δ 7.38-7.18 (m, 2H), 7.13-7.01 (m, 2H), 4.80-4.67 (m, 2H) 4.30-4.15 (m, 1H), 4.13-4.01 (m, 1H), 3.96-3.83 (m, 2H), 3.75-3.60 (m, 2H), 3.42-3.11 (m, 9H), 3.06-2.95 (m, 1H), 2.70-2.58 (m, 4H), 2.28-2.01 (m, 4H), 1.88-1.70 (m, 3H), 1.57-1.25 (m, 4H), 1.22-0.95 (m, 18H), 0.92-0.80 (m, 4H), 0.78-0.65 (nm, 1H).

### 2. Preparation of Toxin Intermediates

### (S)-2-((2R,3R)-3-((1S,3S,5S)-2-((3R,4S,5S)-4-((S)-2-((S)-2-(6-(2,5-dicarbonyl-2,5-dihydro-1H-pyrrol-1-yl)-N-methyl hexanamide)-3-methyl butanamide)-N,3-dimethyl butanamide)-3-methoxy-5-methylheptanoyl)-2-azabicyclo[3.1.0]hexane-3-yl)-3-methoxy-2-methylpropanamide)-3-(2-fluorophenyl)propionic acid

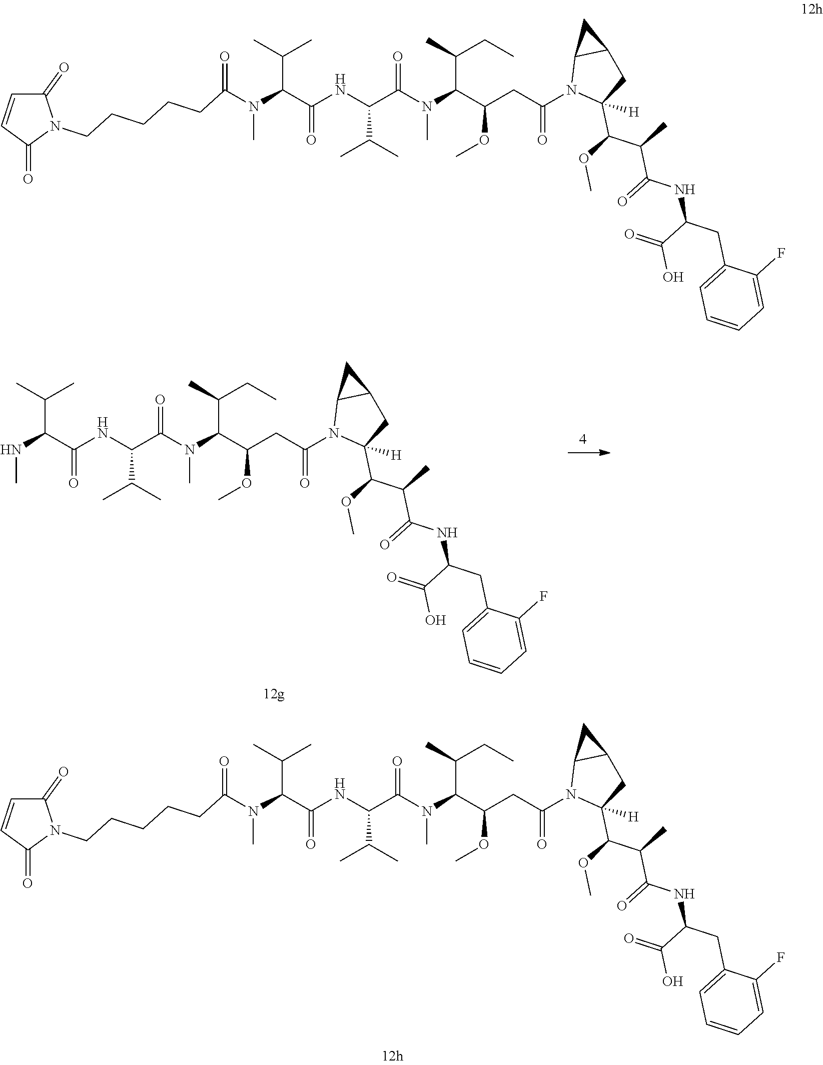 [SMILES: CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@@H](NC)C(C)C)C(C)C ; CC[C@H](C)[C@@H]([C@@H](CC(=O)N1[C@H]2C[C@H]2C[C@H]1[C@H](OC)[C@@H](C)C(=O)N[C@@H](Cc1ccccc1F)C(=O)O)OC)N(C)C(=O)[C@@H](NC(=O)[C@H](C(C)C)N(C)C(=O)CCCCCN1C(=O)C=CC1=O)C(C)C]

The starting material (S)-2-((2R,3R)-3-((1S,3S,5S)-2-((3R,4S,5S)-4-((S)—N,3-dimethyl-2-((S)-3-methyl-2-(methylamino)butanamide)butanamide)-3-methoxy-5-methylheptanoyl)-2-azabicyclo[3.1.0]hexane-3-yl)-3-methoxy-2-methylpropanamide)-3-(2-fluorophenyl) propionic acid 12g (25 mg, 0.033 mmol) was dissolved in dichloromethane (3 mL) and N,N-diisopropylethylamine (0.029 mL, 0.164 mmol) was added. The reaction system was dropwise added with 6-(2,5-dicarbonyl-2,5-dihydro-1H-pyrrol-1-yl)hexanoyl chloride 4b in dichloromethane (11.3 mg, 0.049 mmol) prepared previously under argon atmosphere, in an ice-bath, and the reaction was performed for 3 hours at room temperature. After the reaction, water (5 mL) was added and the mixture was stirred for 20 minutes, until layered, and the organic layer was dried with anhydrous sodium sulfate, filtered and the filtrate was concentrated under reduced pressure. The residues were purified by high performance liquid chromatography to obtain the captioned product compound 12h (7 mg, yellow viscous, with the yield of 22.4%).

MS m/z (ESI): 955.4 [M+1]

¹H NMR (400 MHz, CD₃OD): δ 7.36-7.30 (m, 1H), 7.29-7.21 (m, 1H), 7.17-7.02 (m, 2H), 6.83-6.79 (m, 2H), 4.81-4.71 (m, 2H), 4.69-4.55 (m, 2H), 4.25-4.15 (m, 1H), 4.13-4.04 (m, 1H), 3.96-3.85 (m, 2H), 3.70-3.61 (m, 1H), 3.55-3.46 (m, 3H), 3.40-3.21 (m, 4H), 3.18-3.10 (m, 2H), 3.07-2.96 (m, 4H), 2.67-2.56 (m, 2H), 2.54-2.34 (m, 3H). 2.29-2.17 (m, 2H), 2.10-1.99 (m, 1H), 1.89-1.57 (m, 7H) 1.52-1.28 (m, 6H), 1.21-1.11 (m, 4H), 1.07-0.96 (m, 6H), 0.95-0.81 (m, 12H), 0.80-0.69 (m, 1H).

### 3. Preparation of Antibody-Toxin Conjugate

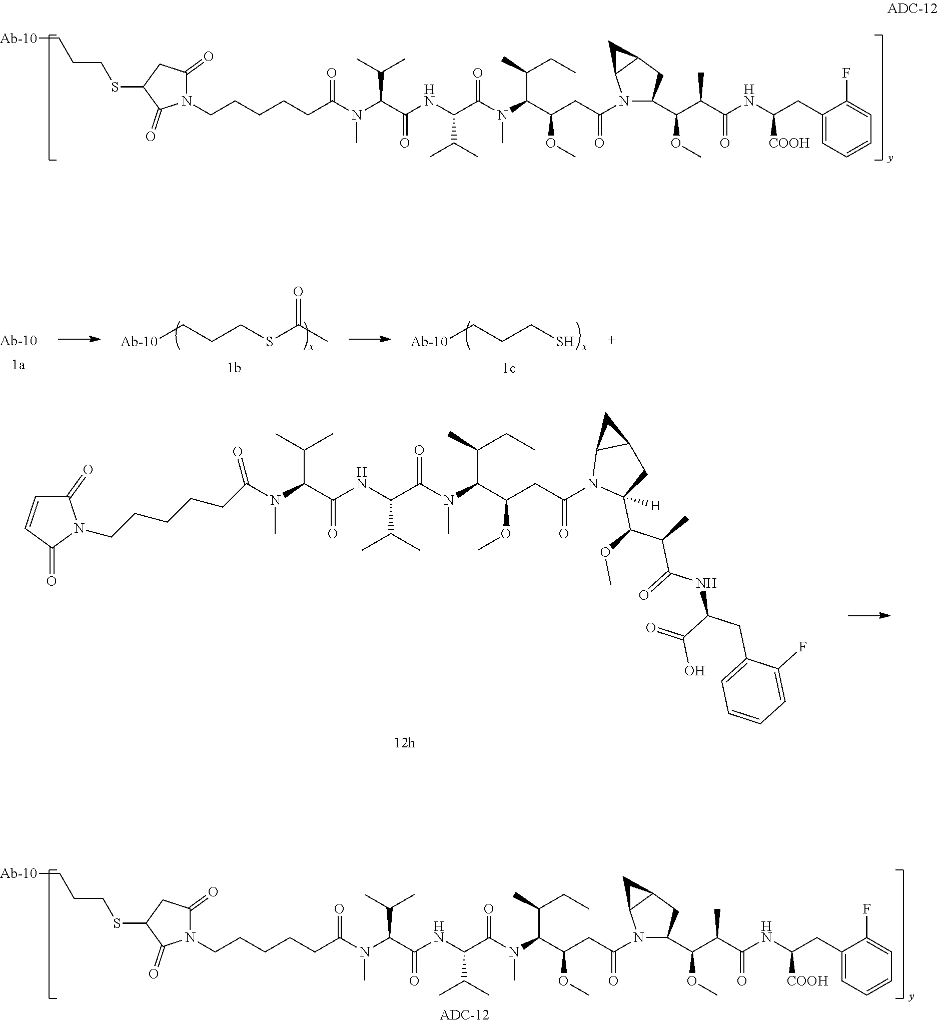

Compound 12h (1.2 mg, 1.2 μmol) was dissolved in acetonitrile (0.3 mL). Ab-10 monoclonal antibody-propylmercaptan 1c solution (6.17 mg/mL, 3.0 mL) was added with shaking for 4 hours at 25° C., and then desalination and purification were done on a Sephadex G25 gel column (Elution phase: 0.05M of PBS solution which pH is 6.5). The captioned product compound ADC-12 in PBS buffer (3.3 mg/mL, 5.0 mL) was obtained by filtration through a 0.2 μm filter under aseptic condition, and then frozen stored at 4° C.

Q-TOF LC/MS: characteristic peak: 148119.6 (M_{Ab}+OD), 149150.5 (M_{Ab}+1D), 150221.1 (M_{Ab}+2D), 151265.1 (M_{Ab}+3D), 152314.3 (M_{Ab}+4D).

Mean value: y=1.6.

With reference to examples 9-18, No. 11-12 ADC compounds were prepared.

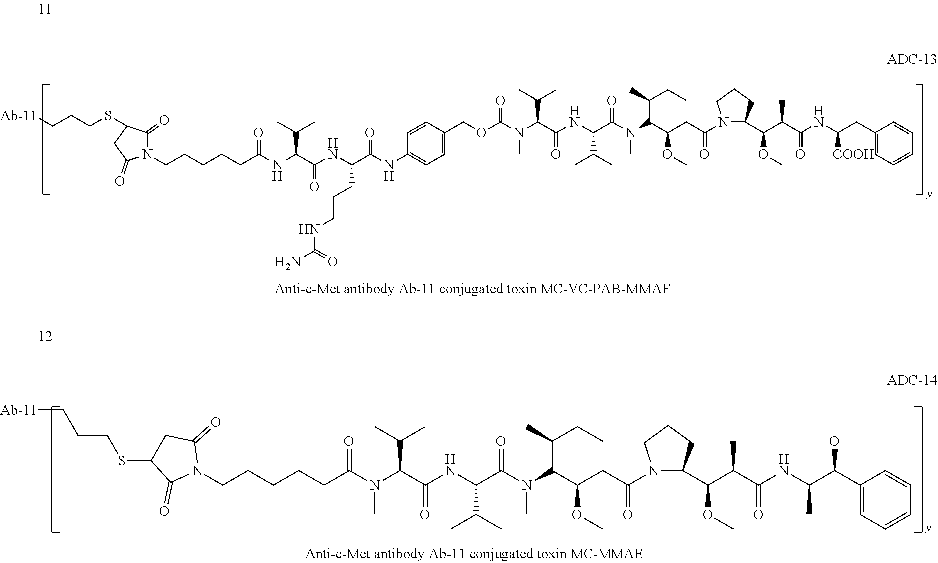

### Example 19. The Inhibitory Effect of Anti-c-Met Antibody Toxin Conjugate (ADC) Molecules on the Proliferation of Hepatic Carcinoma Cells

Test samples: certain antibody compounds of the present invention; the chemical names and preparation methods can be found in the preparation example of each compound.

The inhibitory effect of molecules of the present invention on cell proliferation was tested by the CCK method, and the in vitro activity of ADC molecules of the present invention was evaluated according to IC50.

Cell proliferation was measured using Cell Counting Kit (Dojindo Chemical Technologies, LTD Cat # CK04) (operated according to the instructions). The cells and the corresponding media used are shown in the table below:

| cell line | culture media | Cat. No. |
|---|---|---|
| HepG2 | EMEM + 10% FBS | Cell bank, the Chinese academy of |
| | | science, Cat# TCHu 72 |
| Hep3B | EMEM + 10% FBS | Cell bank, the Chinese academy of |
| | | science, Cat# TCHu106 |
| SK-HEP-1 | EMEM + 10% FBS | Cell bank, the Chinese academy |
| | | of science, Cat# TCHu109 |
| HCCLM3 | DMEM + 10% FBS | Jiangsu Howson pharmaceutical |
| | | co., LTD |
| QGY-7701 | DMEM + 10% FBS | Shanghai Bioleaf biotechnology |
| | | co., LTD |
| SMMC-7721 | DMEM + 10% FBS | Shanghai Bioleaf biotechnology |
| | | co., LTD |
| Bel-7402 | DMEM + 10% FBS | Shanghai Bioleaf biotechnology |
| | | co., LTD |

Experimental Procedures:

During the experiment, 2-3 mL trypsin was introduced to perform digestion for 2-3 min. After the cells were completely digested, the digested cells were eluted by adding 10-15 mL of complete medium, centrifuged at 1000 rpm for 3 min, and the supernatant was discarded. Then, the cells were resuspended by adding 10-20 mL medium to prepare single cell suspensions, and the cell density was adjusted to 4×10⁴ cells/mL. 0.1 mL of the above cell suspension was added to each well of a 96-well cell culture plate, which was incubated at 37° C. in a 5% CO₂ incubator, the media was removed 24 hours later, and 90 μL media containing 2% FBS was added to each well. The samples to be tested were diluted with PBS to different concentration gradients, added with 10 μL per well, and incubated at 37° C. in a 5% CO₂ incubator for 72 hours. 10 μL of CCK8 was added to each well, incubation was continued for another 2 hours in the incubator, the OD450 was detected by a microplate reader (VICTOR 3, PerkinElmer), and data analysis was performed using GraphPad Prism (version 5.0) software.

Experimental Results:

TABLE 8
The inhibitory effects of the molecules of the invention on
proliferation of hepatic carcinoma cells

| | | ADC-1 | | Ab-10 | |
|---|---|---|---|---|---|
| | | | Maximum | | Maximum |
| | | | Inhibition | | Inhibition |
| cell line | tumor type | IC₅₀ (nM) | (%) | IC₅₀ (nM) | (%) |
| HepG2 | hepatic | 81.1 | 25 | 80.90 | 23 |
| | carcinoma | | | | |
| Hep3B | hepatic | 109.4 | 27 | >1000 | 0 |
| | carcinoma | | | | |
| Sk-hep-1 | hepatic | 40.8 | 65 | >1000 | 0 |
| | carcinoma | | | | |
| QGY-7701 | hepatic | 9.1 | 100 | >1000 | 0 |
| | carcinoma | | | | |
| SMMC-7721 | hepatic | 21.7 | 100 | >1000 | 0 |
| | carcinoma | | | | |
| Bel-7402 | hepatic | 0.35 | 83 | 0.50 | 34 |
| | carcinoma | | | | |
| HCCLM3 | hepatic | 1.1 | 85 | 3.20 | 24 |
| | carcinoma | | | | |

Experimental Conclusion:

Based on the above table, the experimental results show that the ADC-1 drug molecule of the present invention has an inhibitory effect on the hepatic carcinoma cell line superior to that of an anti-c-Met antibody of the present invention, indicating that the ADC drug of the present invention has a better inhibitory effect on the proliferation of hepatic carcinoma cells.

### Example 20: The Efficacy of ADC Drugs of the Present Invention on Subcutaneous Xenografted Tumor of Human Hepatic Carcinoma HCCLM3 in Nude Mice

Test sample: certain antibodies and ADC compounds of the present invention; the chemical names and preparation methods can be found in the preparation example of each compound.

Experimental animals: BALB/cA-nude mice, 21-28 days, male, purchased from Shanghai Institute of Materia Medica, Chinese Academy of Sciences. Production license number: SCXK (Shanghai) 2013-0017, No311613700000089. Feeding environment: SPF level.

Preparation of Test Solutions:

ADC-12 was dissolved into a 20 mg/mL solution with water for injection, and each aliquot was stored in a −80° C. refrigerator, then diluted to the corresponding concentrations with 0.1% BSA in saline before use.

An Ab-10 antibody stock solution (antibody concentration of 16.3 mg/mL) was diluted with 0.1% BSA in saline, and each aliquot was stored in a −80° C. refrigerator.

Experimental Procedures:

Nude mice were subcutaneously inoculated with human hepatic carcinoma HCCLM3 cells. Once the tumors were grown to 100-150 mm³, the animals were randomly divided into groups (D0), 10 in each group. The tumor volume was measured 2-3 times per week, the mice were weighed, and the data were recorded.

The tumor volume (V) was calculated according to the formula:

V=1/2×a×b², wherein a and b represent length and width, respectively;

T/C(%)=(T−T₀)/(C−C₀)×100, wherein T and C are tumor volume at the end of the test; T₀ and C₀ are the tumor volume at the start of the test.

Experimental Results:

TABLE 9
Inhibitory test of molecules of the present invention on subcutaneous
xenografted tumors of human hepatic carcinoma HCCLM3 in nude mice

| | | | | | | | | P | | |
|---|---|---|---|---|---|---|---|---|---|---|
| | | Mean | | Mean | | | tumor | value | | |
| | | volume of | | volume of | | % | inhibition | (d21) | | |
| Compound | admini- | tumor (mm³) | | tumor (mm³) | | T/C | rate | (vs | partial | complete |
| Groups | stration | D0 | SEM | D21 | SEM | D21 | D21% | blank) | regression | regression |
| Solvent | D0 | 125.3 | ±4.0 | 2367.4 | ±193.3 | — | — | — | 0 | 0 |
| ADC-12 | D0 | 125.5 | ±3.1 | 1556.6 | ±99.0 | 64 | 36 | 0.002 | 0 | 0 |
| (1 mg/kg) | | | | | | | | | | |
| ADC-12 | D0 | 123.3 | ±3.8 | 425.8 | ±78.6 | 13 | 87 | **0.000 | 1 | 0 |
| (3 mg/kg) | | | | | | | | | | |
| ADC-12 | D0 | 121.5 | ±5.7 | 27.2 | ±12.8 | −78 | 178 | **0.000 | 3 | 6 |
| (10 mg/kg) | | | | | | | | | | |
| Ab-10 | D0,3,7,1 | 123.9 | ±3.5 | 1565.1 | ±150.5 | 64 | 36 | 0.004 | 0 | 0 |
| (10 mg/kg) | 0,14,17 | | | | | | | | | |

D0: first administration time; P value, compared with solvent;
**P < 0.01; all use Student's t test; number of mice at the start of the test: n = 10;
short line — indicates that the data is empty.

Experimental Conclusion:

Based on the data in the above table, ADC-12 (1, 3, 10 mg/kg, iv, D0) inhibited the growth of subcutaneous xenografted tumors (c-Met-expressing human hepatic carcinoma HCCLM3) in nude mice in a dose-dependent manner; tumor inhibition rates were 36%, 87%, and 178% (D21) respectively, 1 out of 10 tumors exhibited partial regression in 3 mg/kg dose group, 3 out of 10 tumors exhibited partial regression and 6 out of 10 tumors exhibited complete regression in 10 mg/kg dose group (D21). By 43 days after the first administration (D42), there were still 7 out of 10 tumors showing complete regression in the 10 mg/kg dose group. As for the Ab-10 antibody stock solution (10 mg/kg, IV, twice per week for 6 times), the tumor inhibition rate against HCCLM3 was 36%; tumor-bearing mice tolerated the above drugs well, and symptoms such as loss of weight were not observed. In comparison, ADC-12 was significantly more effective against HCCLM3 than Ab-10 antibody stock solution.

Therefore, ADC-12 had a significant inhibitory effect on subcutaneous xenograft tumors of c-Met human hepatic carcinoma HCCLM3 in nude mice, effectively inhibited tumor growth, caused partial or complete regression of tumors, and the tumor inhibition rate was increased along with the increase of dosage, inhibitory effect was more significant; Ab-10 antibody stock solution significantly inhibited HCCLM3 and effectively inhibited tumor growth. With the same dosage, ADC-12 was significantly more effective against HCCLM3 than Ab-10 antibody stock solution.

### Example 21. Anti-Tumor Effect of ADC Drugs of the Present Invention on LI-03-0022 HCC Patient-Derived Tumor Transplantation (PDX) Model in BALB/c Nude Mice

Test sample: certain antibodies, ADC compounds of the present invention, the chemical names and preparation methods can be found in the preparation example of each compound.

Experimental animals: BALB/cA-nude mice, 6-8 weeks, 18 females, purchased from Shanghai Sippr-BK Lab Animal Co. Ltd., production license number: SCXK (Shanghai) 2013-0016, animal certificate No.: 2008001658891; Feeding environment: SPF level.

Preparation of Test Solutions:

The drug ADC-12 was dissolved into a 20 mg/mL solution with water for injection, and each aliquot was stored in a −80° C. refrigerator, then diluted to the corresponding concentrations with 5% glucose solution before use;

An Ab-10 antibody stock solution (antibody concentration of 16.3 mg/mL) was diluted with 5% glucose solution, each aliquot was stored in a −80° C. refrigerator; then diluted to the corresponding concentration with 5% glucose solution before use.

The particular preparation method is shown in the table below:

| | Packing | | concentration | |
|---|---|---|---|---|
| compound | size | preparation method | (mg/mL) | storage |
| Solvent | | 5% glucose solution | — | 4° C. |
| ADC-12 | 40 mg/vial | 2.0 mL sterile water was injected into a vial, | 20.0 | −80° C. |
| | | and ADC-12 powder was slowly added; the | | |
| | | mixture was gently stirred until a 20 mg/mL | | |
| | | clear stock solution was obtained; the stock | | |
| | | solution was divided into 20 aliquots which | | |
| | | were stored at −80° C. (0.1 mL/vial). | | |
| ADC-12 | 20 mg/mL | the stored stock solution (0.1 mL, 20 mg/mL) | 1.0 | RT |
| injection | | was diluted with 1.9 mL of 5% | | |
| solution | | glucose solution; the solution was freshly | | |
| | | prepared at the time of each injection before | | |
| | | use. | | |
| Ab-10 | 16.3 mg/mL, | Ab-10 antibody solution was evenly divided | 5.0 | RT |
| antibody | 5 mL/vial | into several aliquots and stored at −80° C.; | | |
| solution | | 0.613 mL stored stock solution was diluted | | |
| | | with 1.386 mL of 5% glucose solution; the | | |
| | | solution was freshly prepared at the time of | | |
| | | each injection before use. | | |
| Ab-10 | 16.3 mg/mL, | Ab-103 antibody solution was evenly | 1.0 | RT |
| antibody | 5 mL/vial | divided into several aliquots and stored at −80° C.; | | |
| solution | | 0.12 mL stock solution was diluted | | |
| | | with 1.836 mL of 5% glucose solution the | | |
| | | solution was freshly prepared at the time of | | |
| | | each injection before use. | | |

Establishment of LI-03-0022 HCC PDX Tumor Model:

An LI-03-0022 HCC (hepatic cellular cancer, hepatic carcinoma) PDX tumor model (patient-derived tumor xenograft model, PDX) was originally established on a clinical tissue sample surgically resected from patients with hepatic carcinoma (from Shanghai Oriental Hepatobiliary Hospital) and was implanted in nude mice and defined as generation 0 (P0). Implantation of tumor generation 0 (P0) was defined as generation 1 (P1); the generation was thus defined according to the order continuously implanted in nude mice. FP3 tumors were recovered from P2T patients, the next generation from FP5 was defined as FP6, and so on; FP5 tumor tissue was used for this study.

Experimental Procedure:

(1) Tumor implantation: Each mouse was s.c. implanted with LI-03-0022 FP5 tumor sections (about 30 mm³) on the right side to develop tumors, and 32 days after tumor implantation, the mean tumor size was close to 183.20 mm³. Since then, treatment was started with 6 tumor-bearing mice in each group, and the experimental procedure for mice was carried out according to the predetermined protocol in the experimental design of the following table:

| | | | | administration | | | |
|---|---|---|---|---|---|---|---|
| | | | dosage | volume, | concentration | administration | administration |
| Group | N^{a} | treatment | mg/kg | mL/kg^{b} | mg/mL | mode | time ^{c} |
| 1 | 6 | solvent | — | 10 | — | iv | BIW × 2 |
| | | | | | | | weeks |
| 2 | 6 | ADC-12 | 10 | 10 | 1.0 | iv | BIW × 2 |
| | | injection | | | | | weeks |
| | | solution | | | | | |
| 3 | 6 | Ab-10 | 10 or | 10 | 1.0 | iv | BIW × 2 |
| | | | 50 | | 5.0 | | weeks |
| | | | | | | | 1^{st}-2^{nd} dosing, |
| | | | | | | | 10 mg/kg; |
| | | | | | | | 3^{rd}-4^{th} dosing, |
| | | | | | | | 50 mg/kg |

Note:
^{a}N is the number of animals in each group;
^{b}the administration volume was adjusted based on 10 μL/g body weight;
^{c} BIW is twice a week; iv is intravenously;

After 2 hours, the last administration was performed, blood samples were collected from all the mice without anticoagulation treatment, and about 50 μL of serum was collected for PK analysis. At the end of the study, tumor samples were collected from 2 animals from the solvent group and 2 animals from the sample group. Animals were divided into two groups: one for FFPE (Formalin-Fixed and Parrffin-Embedded tissue was referred as FFPE sample) and IHC (Immunohistochemistry); the other for Frozen in liquid nitrogen.

(2) Observation and recording: The tumor volume was measured 2-3 times per week, the mice were weighed, and the data were recorded.

(3) Tumor measurement and endpoint

The endpoint was mainly dependent on whether the tumor growth was delayed or whether the mouse could be cured. The tumor volume was measured twice a week with a caliper in two dimensions (in mm³);

The tumor volume (V) was calculated as:

V=0.5×a×b², wherein a and b represent the long and short diameter of the tumor, respectively;

The tumor volume was used to calculate T-C value, T/C value, T-C value by T (the mean time required for the tumor in treatment group to reach 1000 mm³, in days) and C (the mean time required for the tumor in control group to reach the same size, in days); the T/C value (percentage) was used as an indicator of antitumor efficacy, in particular T=Tᵢ/T₀, C=Cᵢ/C₀, Tᵢ is the mean tumor volume of the treatment group on a certain day, T₀ is the mean tumor volume of the treatment group at the beginning of treatment, Cᵢ is the mean tumor volume of the solvent control group at the same time as Tᵢ, and V₀ is the mean tumor volume of the solvent group at the beginning of treatment.

(4) Data analysis: Summary statistics, including mean and standard error (SEM), volumetric analysis of differences in tumor volume between different groups, and volumetric analysis of drug interactions after the last administration (day 14 after grouping) performed by the data obtained at optimal treatment time point, one-way variance analysis was performed to compare tumor volume and tumor weight between groups; when non-significant F-statistic was obtained (p=0.061, treatment variance vs. error variance), Dunnett's T (double-sided) inter-group comparison was performed; all data were analyzed using SPSS 17.0, P<0.05 was considered statistically significant.

Experimental Results:

TABLE 10-1
The change of tumor volume over time

| | tumor volume (mm³)^{a} | | | |
|---|---|---|---|---|
| | | ADC-12 injection | Ab-10 | |
| | | solution | 1st-2nd | 10 mg/kg |
| Day^{b} | solvent | 10 mg/kg | 3rd-4th | 50 mg/kg |

| | | | |
|---|---|---|---|
| 0 | 184 ± 32 | 184 ± 28 | 164 ± 31^{c} |
| 3 | 351 ± 62 | 242 ± 37 | 293 ± 52 |
| 7 | 606 ± 98 | 155 ± 29 | 569 ± 85 |
| 10 | 879 ± 193 | 107 ± 18 | 719 ± 108 |
| 14 | 1,605 ± 367 | 52 ± 12 | 1,067 ± 152 |
| 17 | — | 41 ± 11 | 1,725 ± 195 |
| 21 | — | 29 ± 10 | 1,896 ± 283 |
| 24 | — | 16 ± 8 | — |
| 28 | — | 7 ± 3 | — |
| 31 | — | 6 ± 3 | — |
| 34 | — | 4 ± 2 | — |
| 37 | — | 4 ± 2 | — |
| 41 | — | 14 ± 9 | — |
| 42 | | 15 ± 9 | — |

^{a}is mean ± standard error;
^{b}is the number of days after beginning the treatment;
^{c}is n = 5;
short line, “—”, means that the animals in the corresponding group were sacrificed at this time, no data were obtained.

TABLE 10-2
Analysis of the inhibitory effect of molecules of the present
invention on tumor growth of human hepatic carcinoma
subcutaneous xenograft tumor in BALB/c nude mice

| | Day 14 | | | | | |
|---|---|---|---|---|---|---|
| | | | T-C | Day 21 | | |
| | tumor | | (day) | | tumor | |
| | size | T/C^{b} | at 1000 | p | size | p |
| Sample | (mm³)^{a} | (%) | mm³ | value | (mm³)^{a} | value^{c} |
| solvent | 1,605 ± 367 | — | — | — | — | — |
| ADC-12 | 52 ± 12 | 3.25 | >32 | 0.001 | 29 ± 10 | — |
| injection | | | | | | |
| solution | | | | | | |
| (10 mg/kg) | | | | | | |
| Ab-10 | 1,067 ± 152 | 74.47 | 3 | 0.252 | 1,896 ± 283 | <0.001 |
| (1^{st}-2^{nd}, | | | | | | |
| 10 mg/kg; | | | | | | |
| 3^{rd}-4^{th}, | | | | | | |
| 50 mg/kg)^{d} | | | | | | |

a is mean±standard error; b is tumor growth inhibition, calculated by dividing the mean tumor volume of the treatment group by the mean tumor volume of the control group, T/C must be less than or equal to 50%; c is the p value calculated on the basis of tumor size; d means 3-5 tumor-bearing animals in each group.

TABLE 10-3
Immunohistochemistry (IHC) of c-Met staining

| No. | PDX model | IHC score |
|---|---|---|
| 1 | LI-03-0010 (negative control) | 0 |
| 2 | LI-03-0022 | 2+ |

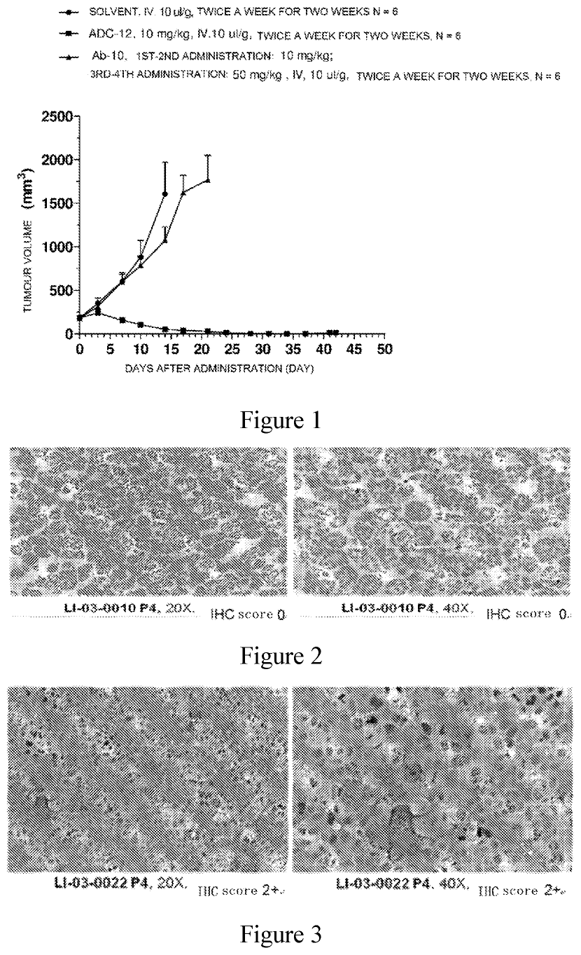

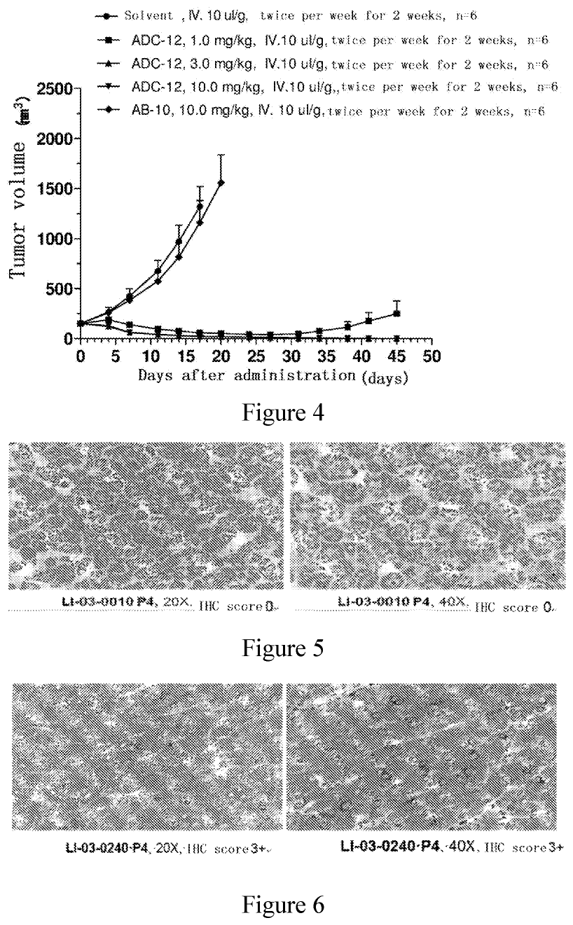

The particular staining area is shown in FIG. 2 and FIG. 3 of the specification.

Note: 0 means unstained, + means light staining, ++ means medium staining, +++ means dark staining; the entire section was read under microscope, the percentage of different staining intensity in cells was determined by visual evaluation, and the H score was calculated as 1×(% of +cells)+2×(% of ++cells)+3×(% of +++cells); then the scoring standard was used to evaluate the figure: the score of 0-0.3 is weakly positive, 0.3-1.5 is moderate positive and 1.5-3 is strongly positive.

Experimental Conclusion:

The results of the above table demonstrate that the ADC drug of the present invention is significantly stronger than the antibody for inhibiting the proliferation of hepatic carcinoma cells; treatment with ADC-12 injection solution resulted in significant antitumor activity with a mean tumor volume of 52.3 mm³ (T/C value=3.25%, p=0.001), when compared with the solvent group; the tumor growth volume was controlled within 1000 mm³ and delayed by 32 days; however, treatment with Ab-10 antibody solution resulted in only minimal antitumor activity, the mean tumor size was controlled within 1,067 mm³ (T/C value=74.47%) and delayed by 3 days; there was no statistically significant difference when compared with the solvent group (p=0.252). The LI-03-0022 HCC PDX model had an impression score of 2+ for c-Met protein expression, indicating that the expression level of c-Met protein in the model is strongly positive and can be used for further research in vivo. Therefore, the ADC drug of the present invention had significant antitumor activity in a LI-03-0022 HCC patient-derived tumor transplantation (PDX) model study, and was well tolerated in tumor-bearing animals.

### Example 22. Anti-Tumor Effect of ADC Drug of the Present Invention on a LI-03-0240 HCC Patient-Derived Tumor Transplantation (PDX) Model in BALB/c Nude Mice

Test sample: certain antibodies and ADC compounds of the present invention; the chemical names and preparation methods can be found in the preparation example of each compound.

Experimental animals: BALB/cA-nude mice, 6-8 weeks, 30 females, purchased from Shanghai Sippr-BK Lab Animal Co. Ltd., production license number: SCXK (Shanghai) 2013-0016, animal certificate No.: 2008001658004; Feeding environment: SPF level.

Preparation of Test Solutions:

The drug ADC-12 lyophilized powder was dissolved as 20 mg/mL solution with water for injection, and each aliquot was stored in a −80° C. refrigerator, then diluted to corresponding concentrations with 5% glucose solution before use;

An Ab-10 antibody stock solution (concentration of 16.3 mg/mL) was diluted with 5% glucose solution, each aliquot was stored in a −80° C. refrigerator; then diluted to corresponding concentrations with 5% glucose solution before use.

The particular preparation method is shown in the table below:

| | packing | | concentration | |
|---|---|---|---|---|
| compound | size | preparation method | mg/mL | storage |
| solvent | — | 5% glucose solution | — | RT |
| ADC-12 | 40 mg/vial | 2.0 mL sterile water was injected into vial, and | 20 | −80° C. |
| | | ADC-12 lyophilized powder was slowly added; the | | |
| | | mixture was gently stirred until 20 mg/mL clear stock | | |
| | | solution was obtained; the stock solution was divided | | |
| | | into 20 aliquots which were stored at −80° C. | | |
| ADC-12 | 20 mg/mL | 0.14 mL ADC-12 was added into a vial, and diluted | 1 | RT |
| injection | | with 2.66 mL 0.5% glucose solution to obtain 1 mg/mL | | |
| solution | | working solution. | | |
| ADC-12 | 1 mg/mL | 0.2 mL ADC-12 injection solution was added into a | 0.1 | RT |
| injection | | vial, and diluted with 1.8 mL 0.5% glucose solution | | |
| solution | | to obtain 0.1 mg/mL working solution. The solution | | |
| | | was prepared before each administration. | | |
| ADC-12 | 1 mg/mL | 0.6 mL ADC-12 injection solution was added into a | 0.3 | RT |
| injection | | vial, and diluted with 1.4 mL 0.5% glucose solution | | |
| solution | | to obtain 0.3 mg/mL working solution. The solution | | |
| | | was prepared before each administration. | | |
| Ab-10 | 16.3 mg/mL, | Ab-10 antibody solution was evenly divided into | 1 | RT |
| antibody | 5 mL/vial | several aliquots and stored at −80° C. | | |
| solution | | 0.12 mL Ab-10 antibody solution was diluted with | | |
| | | 1.836 mL of 5% glucose solution to obtain 1 mg/mL | | |
| | | working solution. The solution was prepared before | | |
| | | each administration. | | |

Establishment of LI-03-0240 HCC PDX Tumor Model:

An LI-03-0240 HCC (hepatic cellular cancer, hepatic carcinoma) PDX tumor model (patient-derived tumor xenograft model, PDX) was originally established on clinical tissue sample surgically resected from patients with hepatic carcinoma (from Shanghai Oriental Hepatobiliary Hospital) which was implanted in nude mice and defined as generation 0 (P0). Implantation of tumor generation 0 (P0) was defined as generation 1 (P1); the generation was thus defined according to the order continuously implanted in nude mice. FP3 tumors were recovered from P2T patients, the next generation from FP5 was defined as FP6, and so on; FP5 tumor tissue was used for this study.

Experimental Procedure:

(1) Tumor implantation: Each mouse was s.c. implanted with LI-03-0240 FP5 tumor sections (about 30 mm³) on the right side to develop tumors, and 15 days after tumor implantation, the mean tumor size was close to 151.79 mm³. Since then, treatment was started with 6 tumor-bearing mice in each group, and the experimental procedure for mice was carried out according to the predetermined protocol in the experimental design of the following table

| | | | | administration | | | |
|---|---|---|---|---|---|---|---|
| | | | dosage | volume, | concentration | administration | administration |
| group | N^{a} | treatment | mg/kg | mL/kg^{b} | mg/mL | mode | time^{c} |
| 1 | 6 | solvent | — | 10 | — | iv | BIW × 2 |
| | | | | | | | weeks |
| 2 | 6 | ADC-12 | 1 | 10 | 0.1 | iv | BIW × 2 |
| | | injection | | | | | weeks |
| | | solution | | | | | |
| 3 | 6 | ADC-12 | 3 | 10 | 0.3 | iv | BIW × 2 |
| | | injection | | | | | weeks |
| | | solution | | | | | |
| 4 | 6 | ADC-12 | 10 | 10 | 1.0 | iv | BIW × 2 |
| | | injection | | | | | weeks |
| | | solution | | | | | |
| 5 | 6 | Ab-10 | 10 | 10 | 1.0 | iv | BIW × 2 |
| | | | | | | | weeks |

Note:
^{a}N is the number of animals in each group;
^{b}the dosage volume was adjusted based on 10 μL/g body weight;
^{c}BIW is twice a week; iv is intravenously;

After 2 hours, the last administration was performed, blood samples were collected from all the mice without anticoagulation treatment, and about 50 μL of serum was collected for PK analysis; at the end of the study, tumor samples were collected from 2 animals from the solvent group and 2 animals from the sample group. Animals were divided into two groups: one for FFPE (Formalin-Fixed and Parrffin-Embedded tissue was referred as FFPE sample) and IHC (Immunohistochemistry); the other for Frozen in liquid nitrogen.

(2) Observation and recording: The tumor volume was measured 2-3 times per week, the mice were weighed, and the data were recorded.

(3) Tumor measurement and endpoint

The endpoint was mainly dependent on whether the tumor growth was delayed or whether the mouse could be cured. The tumor volume was measured twice a week with a caliper in two dimensions (in mm³);

The tumor volume (V) was calculated as:

V=0.5×a×b², wherein a and b represent length and width, respectively;

The tumor volume was used to calculate T-C value, T/C value, T-C value by T (the mean time required for the tumor in treatment group to reach 1000 mm³, in days) and C (the mean time required for the tumor in control group to reach the same size, in days); the T/C value (percentage) was used as an indicator of antitumor efficacy, in particular T=Tᵢ/T₀, C=Cᵢ/C₀, Tᵢ is the mean tumor volume of the treatment group on certain day, T₀ is the mean tumor volume of the treatment group at the beginning of treatment, Cᵢ is the mean tumor volume of the solvent control group at the same time as Tᵢ, and V₀ is the mean tumor volume of the solvent group at the beginning of treatment.

(4) Data analysis: Summary statistics, including mean and standard error (SEM), statistic analysis of differences in tumor volume between different groups, and data analysis of drug interactions after the last administration (day 17 after grouping) performed by the data obtained at optimal treatment time point, one-way variance analysis was performed to compare tumor volume and tumor weight between groups; when non-significant F-statistic was obtained (p<0.001, treatment variance vs. error variance), Games-Howell inter-group comparison was performed; all data were analyzed using SPSS 17.0, P<0.05 was considered statistically significant.

Experimental Results:

TABLE 11-1
the change of tumor volume over time

| | tumor volume (mm³)^{a} | | | | |
|---|---|---|---|---|---|
| | | ADC-12 | ADC-12 | ADC-12 | |
| | | injection | injection | injection | |
| | | solution | solution | solution | Ab-10 |
| Day^{b} | solvent | 1.0 mg/kg | 3.0 mg/kg | 10 mg/kg | 10 mg/kg |
| 0 | 151 ± 20 | 152 ± 10 | 151 ± 17 | 152 ± 27 | 152 ± 16 |
| 4 | 268 ± 47 | 189 ± 10 | 121 ± 17 | 129 ± 27 | 259 ± 22 |
| 7 | 425 ± 74 | 140 ± 10 | 60 ± 9 | 63 ± 19 | 384 ± 43 |
| 11 | 677 ± 105 | 96 ± 10 | 41 ± 8 | 43 ± 16 | 574 ± 91 |
| 14 | 972 ± 162 | 76 ± 9 | 31 ± 5 | 31 ± 11 | 815 ± 141 |
| 17 | 1,320 ± 201 | 60 ± 7 | 22 ± 4 | 24 ± 9 | 1,159 ± 221 |
| 20 | — | 53 ± 6 | 20 ± 4 | 20 ± 8 | 1,556 ± 280 |
| 24 | — | 43 ± 7 | 16 ± 4 | 15 ± 6 | — |
| 27 | — | 41 ± 6 | 8 ± 2 | 14 ± 5 | — |
| 31 | — | 50 ± 11 | 3 ± 2 | 9 ± 5 | — |
| 34 | — | 75 ± 28 | 2 ± 1 | 7 ± 4 | — |
| 38 | — | 114 ± 56 | 0 ± 0 | 4 ± 3 | — |
| 41 | — | 177 ± 84 | 0 ± 0 | 2 ± 2 | — |
| 45 | — | 248 ± 130 | 0 ± 0 | 1 ± 1 | — |

^{a}is mean ± standard error;
^{b}is the number of days after beginning the treatment;
^{c}means n = 5;
short line, “—” means that the animals in corresponding group were sacrificed at this time, no data were obtained.

TABLE 11-2
Analysis of the inhibitory effect of molecules of the present
invention on tumor growth of human hepatic carcinoma
subcutaneous xenograft tumor in BALB/c nude mice

| | Day 17 | | | Day 21 | | |
|---|---|---|---|---|---|---|
| | tumor | | T-C (day) | | tumor | |
| | size | T/C^{b} | value | p | size | p |
| sample | (mm³)^{a} | (%) | at 1000 mm³ | value^{c} | (mm³)^{a} | value^{c} |
| solvent | 1,320 ± 201 | — | — | — | — | — |
| ADC-12 injection | 60 ± 7 | 4.51 | >31 | 0.008 | 53 ± 6 | 0.011 |
| solution (1 mg/kg) | | | | | | |
| ADC-12 injection | 22 ± 4 | 1.66 | >31 | 0.007 | 20 ± 4 | 0.010 |
| solution (3 mg/kg) | | | | | | |
| ADC-12 injection | 24 ± 9 | 1.84 | >31 | 0.007 | 20 ± 8 | 0.010 |
| solution (10 mg/kg) | | | | | | |
| Ab-10 (10 mg/kg) | 1,159 ± 221 | 87.75 | 1 | 0.980 | 1,556 ± 280 | — |

^{a}is mean ± standard error;
^{b}is tumor growth inhibition, calculated by dividing the mean tumor volume of the treatment group by the mean tumor volume of the control group, T/C must be less than or equal to 50%;
^{c}is the p value calculated on the basis of tumor size;
^{d}means 3-5 tumor-bearing animals in each group.

TABLE 11-3
Immunohistochemistry (IHC) of c-Met staining

| No. | PDX model | IHC score |
|---|---|---|
| 1 | LI-03-0010 (negative control) | 0 |
| 2 | LI-03-0240 | 3+ |

The particular staining area is shown in FIG. 5 and FIG. 6 of the specification.

Note: 0 means unstained, + means light staining, ++ means medium staining, +++ means dark staining; the entire section was read under microscope, the percentage of different staining intensity in cells was determined by visual evaluation, and the H score was calculated as 1×(% of +cells)+2×(% of ++cells)+3×(% of +++cells); then the scoring standard was used to evaluate the figure: the score of 0-0.3 is weakly positive, 0.3-1.5 is moderate positive and 1.5-3 is strongly positive.

Experimental Conclusion:

The results of the above table demonstrate that the ADC drug of the present invention is significantly stronger than the antibody for inhibiting the proliferation of hepatic carcinoma cells; treatment with ADC-12 injection solution resulted in significant antitumor activity with a mean tumor volume of 52.3 mm³ (T/C value=3.25%, p=0.001), when compared with the solvent group; the tumor growth volume was controlled within 1000 mm³ and delayed by 32 days; however, treatment with Ab-10 antibody solution resulted in only minimal antitumor activity, the mean tumor size was controlled within 1,067 mm³ (T/C value=74.47%) and delayed by 3 days; there was no statistically significant difference when compared with the solvent group (p=0.252). The LI-03-0240 PDX model had an impression score of 3+ for c-Met protein expression, indicating that the expression level of c-Met protein in the model is strongly positive and can be used for further research in vivo. Therefore, the ADC drug of the present invention had significant antitumor activity in a LI-03-0240 HCC patient-derived tumor transplantation (PDX) model study, and was well tolerated in tumor-bearing animals.

1-36. (canceled)

37. A method of treating hepatic carcinoma in a subject in need thereof, the method comprising administering to the subject an antibody-cytotoxic drug conjugate or a pharmaceutically acceptable salt or solvate thereof, wherein the antibody-cytotoxic drug conjugate has a structure of formula (I):
Ab-[(L₂)t-L₁-D)]y (I)
wherein:
D is a cytotoxic drug;
L₁ and L₂ are linker units;
t is 0 or 1;
y is 1-8; and
Ab is an antibody or antigen-binding fragment thereof that specifically binds to c-Met receptor, comprising:
an antibody heavy chain variable region comprising an amino acid sequence having HCDR sequences of SEQ ID NO: 6, SEQ ID NO:7 and SEQ ID NO:8, or a mutant sequence thereof; and
an antibody light chain variable region comprising an amino acid sequence having LCDR sequences of SEQ ID NO: 9, SEQ ID NO: 10 and SEQ ID NO: 11, or a mutant sequence thereof.

38. The method of claim 37, wherein the antibody or antigen-binding fragment thereof that specifically binds to c-Met receptor is a chimeric antibody or a humanized antibody, or antigen-binding fragment thereof.

39. The method of claim 38, wherein the antibody or antigen-binding fragment thereof that specifically binds to c-Met receptor is a humanized antibody, wherein the humanized antibody heavy chain variable region comprises heavy chain framework regions having FR1, FR2, FR3 and FR4 of the human germline heavy chain IGHV 3-33*01, or a mutant sequence thereof, and wherein the humanized antibody light chain variable region comprises heavy chain framework regions having FR1, FR2, FR3 and FR4 of the human germline light chain IGKV085 or IGKV4-1*01, or a mutant sequence thereof.

40. The method of claim 39, wherein the humanized antibody comprises a heavy chain variable region having an amino acid sequence selected from the group consisting of SEQ ID NOs: 13, 14 and 15, and comprises a light chain variable region having an amino acid sequence selected from the group consisting of SEQ ID NOs: 16, 17 and 18.

41. The method of claim 37, wherein the antibody or antigen-binding fragment thereof that specifically binds to c-Met receptor comprises a combination of a heavy chain variable region amino acid sequence and a light chain variable region amino acid sequence selected from any one of a) to c):
a) Heavy chain variable region sequence of SEQ ID NO: 13, and light chain variable region sequence of SEQ ID NO: 16;
b) Heavy chain variable region sequence of SEQ ID NO: 14, and light chain variable region sequence of SEQ ID NO: 17; and
c) Heavy chain variable region sequence of SEQ ID NO: 15, and light chain variable region sequence of SEQ ID NO: 18.

42. The method of claim 38, wherein the antibody or antigen-binding fragment thereof that specifically binds to c-Met receptor is a humanized antibody, wherein the heavy chain constant region of the humanized antibody comprises a constant region derived from human IgG1 or a variant thereof, human IgG2 or a variant thereof, human IgG3 or a variant thereof, or human IgG4 or a variant thereof, and wherein the light chain constant region of the humanized antibody comprises a constant region selected from the group consisting of human κ and human λ, or a variant thereof.

43. The method of claim 42, wherein the antibody or antigen-binding fragment thereof that specifically binds to c-Met receptor comprises a full-length heavy chain sequence selected from the group consisting of SEQ ID NOs: 23, 24 and 25 and sequences having at least 90% identity to SEQ ID NOs: 23, 24 or 25, and comprises a full-length light chain sequence selected from the group consisting of SEQ ID NOs: 26, 27 and 28 and sequences having at least 90% identity to SEQ ID NOs: 26, 27 and 28.

44. The method of claim 38, wherein the antibody or antigen-binding fragment thereof that specifically binds to c-Met receptor is a humanized antibody, wherein the humanized antibody comprises a combination of a full-length light chain amino acid sequence and a full-length heavy chain amino acid sequence selected from:
Ab-9, comprising a heavy chain amino acid sequence of SEQ ID NO: 23 and a light chain amino acid sequence of SEQ ID NO: 26;
Ab-10, comprising a heavy chain amino acid sequence of SEQ ID NO: 24 and a light chain amino acid sequence of SEQ ID NO: 27; and
Ab-11, comprising a heavy chain amino acid sequence of SEQ ID NO: 25 and a light chain amino acid sequence of SEQ ID NO: 28.

45. The method of claim 37, wherein the antibody-cytotoxic drug conjugate or pharmaceutically acceptable salt or solvate thereof is administered in a pharmaceutical composition, the pharmaceutical composition comprising the antibody-cytotoxic drug conjugate or pharmaceutically acceptable salt or solvate thereof and at least one pharmaceutically acceptable excipient, diluent or carrier.

46. The method of claim 37, wherein -L₂- comprises formula (-L₂-): 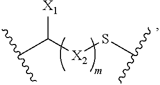
wherein:
X₁ is selected from the group consisting of hydrogen, halogen, hydroxyl, cyano, C₁₋₆ alkyl, C₁₋₆ alkoxy and 3-8 membered cycloalkyl;
X₂ is selected from the group consisting of C₁₋₆ alkyl, 3-8 membered cycloalkyl and 3-8 membered heterocyclyl;
m is 0, 1, 2, 3, 4 or 5; and
S is a sulfur atom.

47. The method of claim 37, wherein D is a cytotoxic agent selected from the group consisting of toxins, chemotherapeutic agents, antibiotics, radioisotopes and nucleolytic enzymes.

48. The method of claim 47, wherein D comprises formula (D): 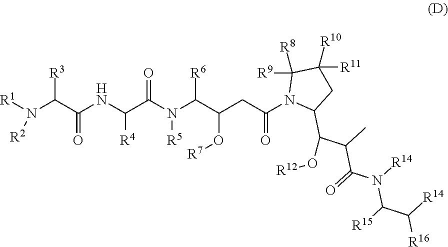
or a tautomer, mesomer, racemate, enantiomer, or diastereomer thereof, or mixture thereof, or a pharmaceutically acceptable salt thereof;
wherein:
R¹, R², R³, R⁴, R⁵, R⁶, and R⁷ are each selected from the group consisting of hydrogen, halogen, hydroxyl, cyano, C₁₋₆ alkyl, C₁₋₆ alkoxy and 3-8 membered cycloalkyl;
R⁸, R⁹, R¹⁰, and R¹¹ are each selected from the group consisting of hydrogen, halogen, C₂₋₆ alkenyl, C₁₋₆ alkyl, C₁₋₆ alkoxy and 3-8 membered cycloalkyl;
or any two of R⁸, R⁹, R¹⁰ and R¹¹ are taken together with the carbon atoms to which they are attached to form a 3-8 membered cycloalkyl, and the rest are each selected from the group consisting of hydrogen, C₁₋₆ alkyl and 3-8 membered cycloalkyl;
R¹² and R¹³ are each selected from the group consisting of hydrogen, C₁₋₆ alkyl and halogen;
R¹⁴ is selected from the group consisting of 6-8 membered aryl and 5-8 membered heteroaryl, wherein the aryl or heteroaryl is optionally further substituted by a substituent selected from the group consisting of hydrogen, halogen, hydroxy, C₁₋₆ alkyl, C₁₋₆ alkoxy and 3-8 membered cycloalkyl;
R¹⁵ is selected from the group consisting of halogen, C₂₋₆ alkenyl, C₁₋₆ alkyl, 3-8 membered cycloalkyl, carboxyl, C₁₋₆ alkyl carbonyl and C₁₋₆ alkoxy carbonyl;
R¹⁶ is selected from the group consisting of hydrogen, halogen, hydroxyl, cyano, alkyl, C₁₋₆ alkoxy and 3-8 membered cycloalkyl.

49. The method of claim 48, wherein L₂ comprises a linker selected from the group consisting of valine-citrulline (Val-Cit), 6-maleimido-caproyl (MC), P-aminobenzyloxycarbonyl (PAB) and 6-maleimido-caproyl-P-aminobenzyloxycarbonyl (MC-PAB).

50. The method of claim 37, wherein D is a maytansinoid.

51. The method of claim 50, wherein L₂ is selected from the group consisting of N-succinimidyl 4-(2-pyridylthio) valerate, N-succinimidyl 4-(N-maleimidomethyl)-cyclohexane-1-carboxylate and N-succinimidyl (4-iodo-acetyl) aminobenzoate.

52. The method of claim 37, wherein D is a camptothecin alkaloid selected from the group consisting of camptothecin (CPT), 10-hydroxy-CPT, Irinotecan, SN-38 and topotecan.

53. The method of claim 52, wherein L₂ is selected from the group consisting of valine-citrulline (Val-Cit), 6-maleimido-caproyl (MC), P-aminobenzyloxycarbonyl (PAB) and 6-maleimido-caproyl-P-aminobenzyloxycarbonyl (MC-PAB).

54. The method of claim 37, wherein the antibody-cytotoxic drug conjugate of formula (I) or the pharmaceutically acceptable salt or solvate thereof is an antibody-cytotoxic drug conjugate of formula (II) or a pharmaceutically acceptable salt or solvate thereof: 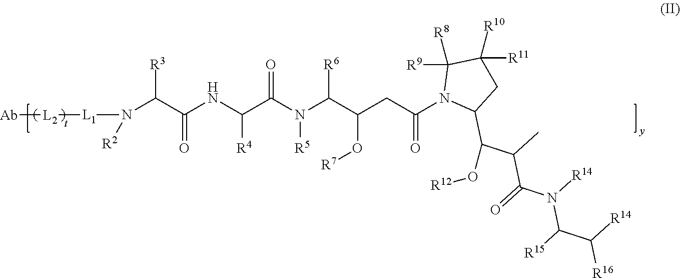
wherein:
R¹, R², R³, R⁴, R⁵, R⁶, and R⁷ are each selected from the group consisting of hydrogen, halogen, hydroxyl, cyano, C₁₋₆ alkyl, C₁₋₆ alkoxy and 3-8 membered cycloalkyl;
R⁸, R⁹, R¹⁰, and R¹¹ are each selected from the group consisting of hydrogen, halogen, C₂₋₆ alkenyl, C₁₋₆ alkyl, C₁₋₆ alkoxy and 3-8 membered cycloalkyl;
or any two of R⁸, R⁹, R¹⁰ and R¹¹ are taken together with the carbon atoms to which they are attached to form a 3-8 membered cycloalkyl, and the rest are each selected from the group consisting of hydrogen, C₁₋₆ alkyl and 3-8 membered cycloalkyl;
R¹² and R¹³ are each selected from the group consisting of hydrogen, C₁₋₆ alkyl and halogen;
R¹⁴ is selected from the group consisting of 6-8 membered aryl and 5-8 membered heteroaryl, wherein the aryl or heteroaryl is optionally further substituted by a substituent selected from the group consisting of hydrogen, halogen, hydroxy, C₁₋₆ alkyl, C₁₋₆ alkoxy and 3-8 membered cycloalkyl;
R¹⁵ is selected from the group consisting of halogen, C₂₋₆ alkenyl, C₁₋₆ alkyl, 3-8 membered cycloalkyl, carboxyl, C₁₋₆ alkyl carbonyl and C₁₋₆ alkoxy carbonyl;
R¹⁶ is selected from the group consisting of hydrogen, halogen, hydroxyl, cyano, alkyl, C₁-6 alkoxy and 3-8 membered cycloalkyl; and
Ab, t, y, L₁, and L₂ are as defined in claim 37.

55. The method of claim 37, wherein the antibody-cytotoxic drug conjugate of formula (I) or the pharmaceutically acceptable salt or solvate thereof is an antibody-cytotoxic drug conjugate of formula (III) or a pharmaceutically acceptable salt or solvate thereof: 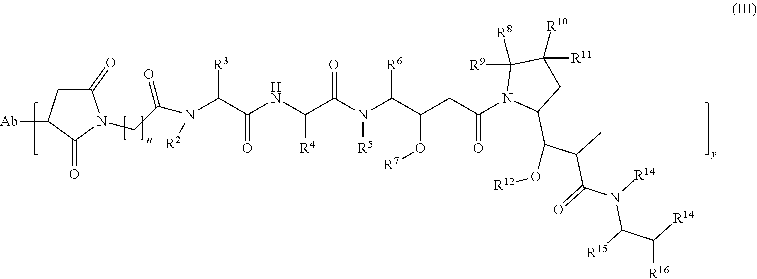
wherein:
R², R³, R⁴, R⁵, R⁶, and R⁷ are each selected from the group consisting of hydrogen, halogen, hydroxyl, cyano, C₁₋₆ alkyl, C₁₋₆ alkoxy and 3-8 membered cycloalkyl;
R⁸, R⁹, R¹⁰, and R¹ are each selected from the group consisting of hydrogen, halogen, C₂₋₆ alkenyl, C₁₋₆ alkyl, C₁₋₆ alkoxy and 3-8 membered cycloalkyl;
or any two of R⁸, R⁹, R¹⁰ and R¹¹ are taken together with the carbon atoms to which they are attached to form a 3-8 membered cycloalkyl, and the rest are each selected from the group consisting of hydrogen, C₁₋₆ alkyl and 3-8 membered cycloalkyl;
R¹² and R¹³ are each selected from the group consisting of hydrogen, C₁₋₆ alkyl and halogen;
R¹⁴ is selected from the group consisting of 6-8 membered aryl and 5-8 membered heteroaryl, wherein the aryl or heteroaryl is optionally further substituted by a substituent selected from the group consisting of hydrogen, halogen, hydroxy, C₁₋₆ alkyl, C₁₋₆ alkoxy and 3-8 membered cycloalkyl;
R¹⁵ is selected from the group consisting of halogen, C₂₋₆ alkenyl, C₁₋₆ alkyl, 3-8 membered cycloalkyl, carboxyl, C₁₋₆ alkyl carbonyl and C₁₋₆ alkoxy carbonyl;
R¹⁶ is selected from the group consisting of hydrogen, halogen, hydroxyl, cyano, alkyl, Cᵢ-6 alkoxy and 3-8 membered cycloalkyl;
Ab and y are as defined in claim 37; and
n is 3, 4, 5 or 6.

56. The method of claim 37, wherein the antibody-cytotoxic drug conjugate of formula (I) or the pharmaceutically acceptable salt or solvate thereof is an antibody-cytotoxic drug conjugate of formula (IV) or a pharmaceutically acceptable salt or solvate thereof: 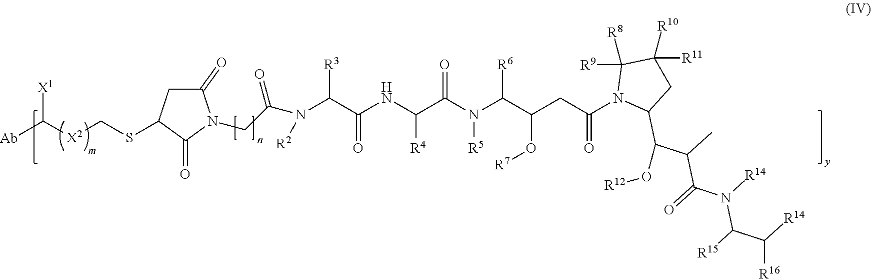
wherein:
R², R³, R⁴, R⁵, R⁶, and R⁷ are each selected from the group consisting of hydrogen, halogen, hydroxyl, cyano, C₁₋₆ alkyl, C₁₋₆ alkoxy and 3-8 membered cycloalkyl;
R⁸, R⁹, R¹⁰, and R¹¹ are each selected from the group consisting of hydrogen, halogen, C₂₋₆ alkenyl, C₁₋₆ alkyl, C₁₋₆ alkoxy and 3-8 membered cycloalkyl;
or any two of R⁸, R⁹, R¹⁰ and R¹¹ are taken together with the carbon atoms to which they are attached to form a 3-8 membered cycloalkyl, and the rest are each selected from the group consisting of hydrogen, C₁₋₆ alkyl and 3-8 membered cycloalkyl;
R¹² and R¹³ are each selected from the group consisting of hydrogen, C₁₋₆ alkyl and halogen;
R¹⁴ is selected from the group consisting of 6-8 membered aryl and 5-8 membered heteroaryl, wherein the aryl or heteroaryl is optionally further substituted by a substituent selected from the group consisting of hydrogen, halogen, hydroxy, C₁₋₆ alkyl, C₁₋₆ alkoxy and 3-8 membered cycloalkyl;
R¹⁵ is selected from the group consisting of halogen, C₂₋₆ alkenyl, C₁₋₆ alkyl, 3-8 membered cycloalkyl, carboxyl, C₁₋₆ alkyl carbonyl and C₁₋₆ alkoxy carbonyl;
R¹⁶ is selected from the group consisting of hydrogen, halogen, hydroxyl, cyano, alkyl, C₁₋₆ alkoxy and 3-8 membered cycloalkyl;
n is 3, 4, 5 or 6;
X₁ is selected from the group consisting of hydrogen, halogen, hydroxyl, cyano, C₁₋₆ alkyl, C₁₋₆ alkoxy and 3-8 membered cycloalkyl;
X₂ is selected from the group consisting of C₁₋₆ alkyl, 3-8 membered cycloalkyl and 3-8 membered heterocyclyl; and
Ab and y are as defined in claim 37.

57. The method of claim 37, wherein the antibody-cytotoxic drug conjugate of formula (I) or the pharmaceutically acceptable salt or solvate thereof is an antibody-cytotoxic drug conjugate of formula (V) or a pharmaceutically acceptable salt or solvate thereof: 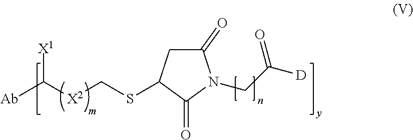
n is 3, 4, 5 or 6;
X₁ is selected from the group consisting of hydrogen, halogen, hydroxyl, cyano, C₁₋₆ alkyl, C₁₋₆ alkoxy and 3-8 membered cycloalkyl;
X₂ is selected from the group consisting of C₁₋₆ alkyl, 3-8 membered cycloalkyl and 3-8 membered heterocyclyl; and
Ab, D, and y are as defined in claim 37.

58. The method of claim 37, wherein the antibody-cytotoxic drug conjugate of formula (I) is selected from the group consisting of: 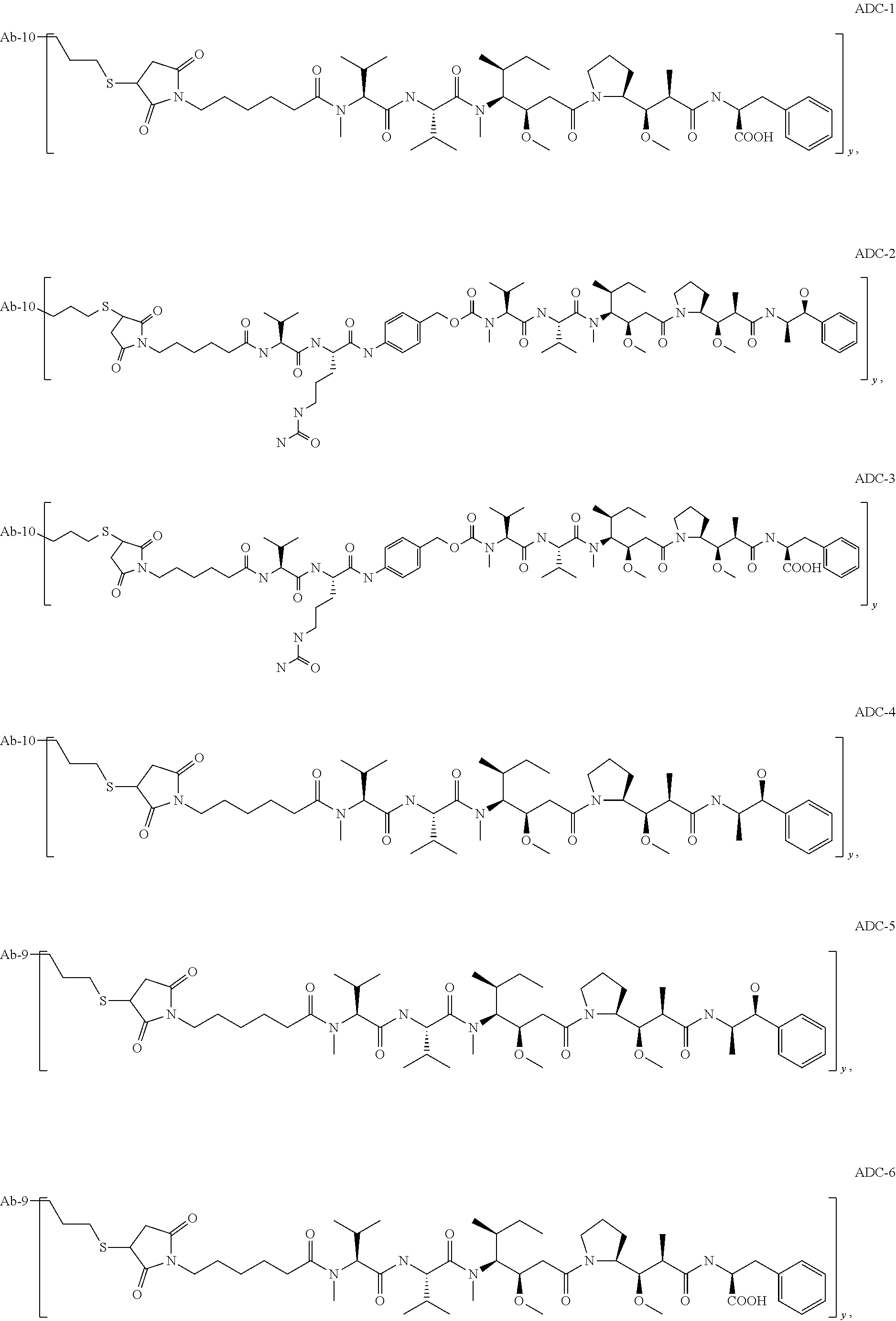 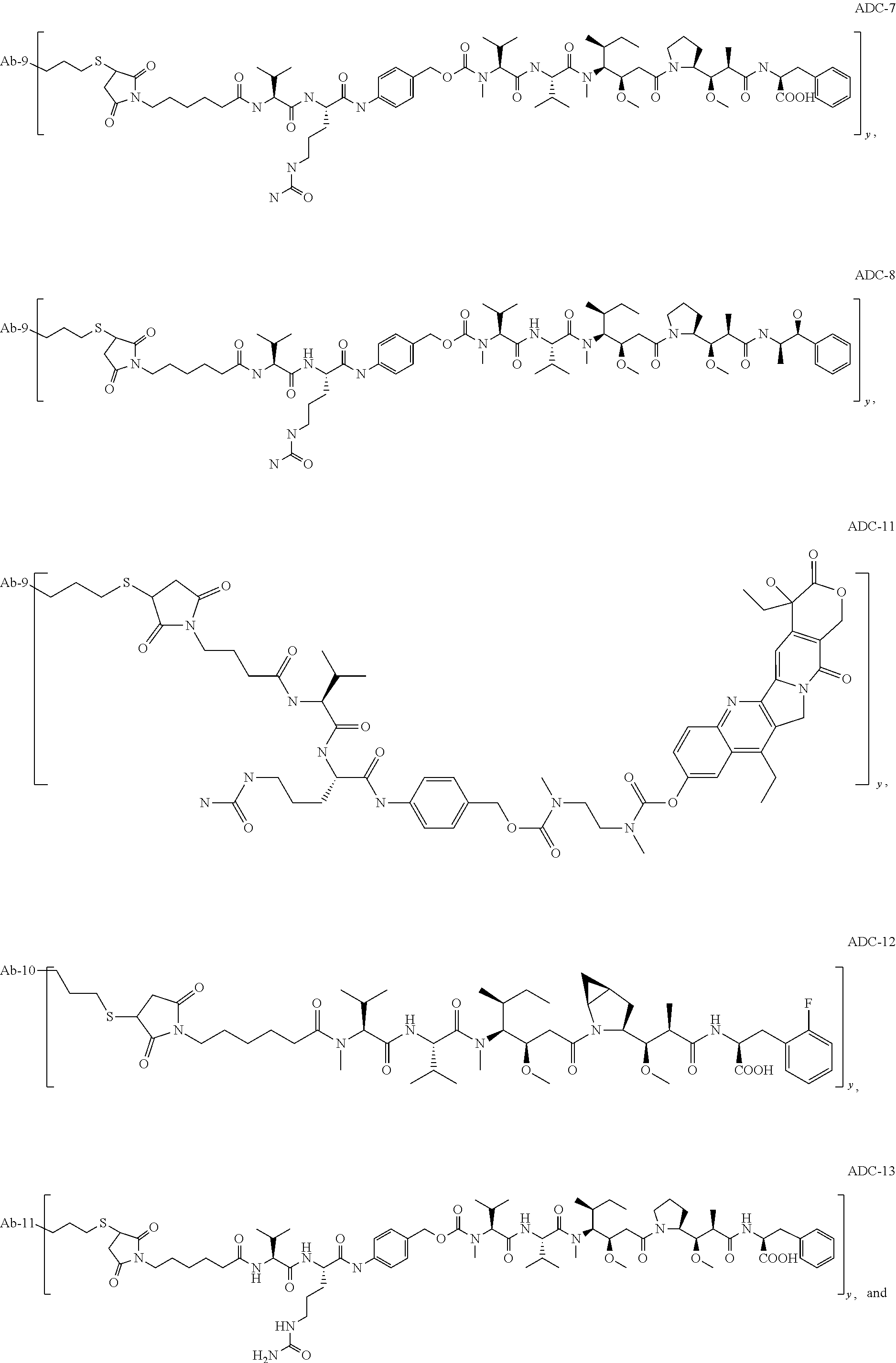 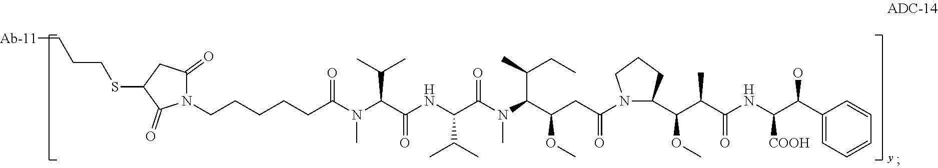
or a pharmaceutically acceptable salt or solvate thereof,
wherein:
Ab-9 is a humanized antibody comprising a heavy chain amino acid sequence of SEQ ID NO: 23 and a light chain amino acid sequence of SEQ ID NO: 26;
Ab-10 is a humanized antibody comprising a heavy chain amino acid sequence of SEQ ID NO: 24 and a light chain amino acid sequence of SEQ ID NO: 27; and
Ab-11 is a humanized antibody comprising a heavy chain amino acid sequence of SEQ ID NO: 25 and a light chain amino acid sequence of SEQ ID NO: 28;
and y is 1, 2, 3, 4, 5, 6, 7 or 8.

59. The method of claim 37, wherein the hepatic carcinoma is c-Met positive hepatic carcinoma or hepatic carcinoma which overexpresses c-Met.
